(19) United States
(12) Patent Application Publication
(10) Pub. No.: US 2010/0093973 A1
(43) Pub. Date: Apr. 15, 2010

(54) POLY(AMINO ACID) COMPOUND HAVING INHIBITORY ACTIVITY ON ABSORPTION OF PHOSPHORUS AND PHOSPHORUS ABSORPTION INHIBITOR

(75) Inventors: Tadakiyo Nakagawa, Kawasaki-shi (JP); Hironobu Tsugeno, Kawasaki-shi (JP); Naomi Matsutani, Kawasaki-shi (JP); Masayuki Sugiki, Kawasaki-shi (JP); Haruko Hirashima, Kawasaki-shi (JP); Tatsuya Okuzumi, Kawasaki-shi (JP); Tatsuya Kasahara, Kawasaki-shi (JP); Wataru Miyanaga, Kawasaki-shi (JP); Takashi Yamamoto, Kawasaki-shi (JP); Masatsugu Noguchi, Kawasaki-shi (JP); Kayo Matsumoto, Kawasaki-shi (JP); Hideyuki Tanaka, Kawasaki-shi (JP); Tomoyuki Konda, Kawasaki-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: AJINOMOTO CO., INC., Tokyo (JP)

(21) Appl. No.: 12/549,219

(22) Filed: Aug. 27, 2009

Related U.S. Application Data
(63) Continuation of application No. PCT/JP2008/053365, filed on Feb. 27, 2008.

(30) Foreign Application Priority Data
Feb. 27, 2007 (JP) 2007-47633

(51) Int. Cl. C07K 2/00

(52) U.S. Cl. 530/300

(57) ABSTRACT

Herein disclosed is a water-soluble compound represented by the following general formula (I) or a salt thereof. The compound is a novel water-soluble compound having an inhibitory effect on the absorption of phosphorus in vivo.

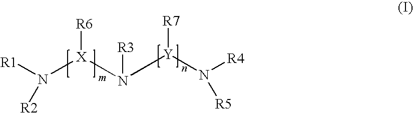

where the structural variables are defined herein.

### TECHNICAL FIELD

The present invention relates to a poly(amino acid) compound having an inhibitory activity on the absorption of phosphorus, which has a polyamine skeleton and which include, for instance, a dendrimer-type compound and an agent for inhibiting the absorption of phosphorus in the living body, which comprises the foregoing compound.

### BACKGROUND OF THE INVENTION

There have been proposed an orally administrable phosphorus absorber which comprises, as an effective component, polylysine or polylysine crosslinked with a crosslinking agent (see Patent Document 1). This Patent Document states that the orally administrable phosphorus absorber is excellent in the effect of absorbing phosphorus, for instance, that excessively ingested, within the digestive tract and excellent in the effect of accelerating the excretion of the absorbed phosphorus into the feces and that the phosphorus absorber would, in particular, permit the relaxation or alleviation of the phosphorus-intake in the patients suffering from a variety of disease caused by, for instance, the excess intake of phosphorus, such as hyper-phosphatemia, renal failure and osteoporosis, and that the phosphorus absorber is useful as a medicinal agent for preventing and treating these diseases.

On the other hand, there has also been reported the results of the study in which dendrimer-type poly(L-lysines) in various generations are synthesized and these poly(L-lysines) are inspected for the characteristic properties as gene-transforming agents (Non-Patent Document 1). In this study, hexamethylene-diamine is used as a starting core, the core is condensed with L-lysine to thus synthesize a dendrimer-type poly(L-lysine), but this Non-Patent Document 1 never discloses whether the resulting poly(L-lysine) shows an effect of inhibiting the absorption of phosphorus in the living body or not.

Patent Document 1: JP-A-2003-33651;

Non-Patent Document 1: Bioconjugate Chem., 2002, 13, pp. 510-517.

### DISCLOSURE OF THE INVENTION

It is an object of the present invention to provide a novel water-soluble compound having an inhibitory effect on the absorption of phosphorus in vivo.

It is another object of the present invention to provide a phosphorus absorption inhibitor which comprises the foregoing novel compound having a phosphorus absorption inhibitory effect.

The present invention has been completed on the basis of such a finding that a compound, which can solve the foregoing problems, can be obtained by condensing, with amino acids or the like, a polyamine serving as a skeleton and having such a specific structure that it has at least three amino-nitrogen atoms in the molecule.

In other words, the present invention herein provides a water-soluble compound represented by the following general formula (I) or a salt thereof:

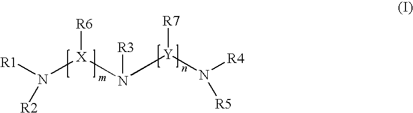

In the formula (I), R1 to R7 may be the same or different and each represent a hydrogen atom, an amidino group, a substituted or unsubstituted aminoalkyl group, a substituted or unsubstituted alkyl group, or a group: Rx-[A]p- (wherein A represents a connecting group; p represents 0 or 1; and Rx represents an amino-carboxylic acid residue, a substituted or unsubstituted aminoalkyl group, a substituted or unsubstituted alkyl group, or a group represented by the following formula (II);

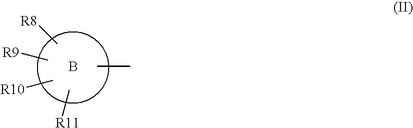

(In Formula (II), B represents an aryl group, a heteroaryl group or a cycloalkyl group, R8 to R11 may be the same or different and each represent a hydrogen atom, an amidino group, an amino group, a substituted or unsubstituted aminoalkyl group, a substituted or unsubstituted alkyl group, or a group: Ry-[A1]q- (wherein A1 represents a connecting group; q represents 0 or 1; and Ry represents an amino-carboxylic acid residue, a substituted or unsubstituted aminoalkyl group, or a substituted or unsubstituted alkyl group))),

X and Y may be the same or different and each represent —N— or —CH—, m and n may be the same or different and each represent an integer ranging from 2 to 7, provided that when m is 2, X represents —CH—, when m is not less than 3, a plurality of groups X—R6 may be the same or different, but is not adjacent to N—R6, and

When n is 2, Y represents —CH— and when n is not less than 3, a plurality of groups Y—R7 may be the same or different, but is not adjacent to N—R7.

The present invention also provides a phosphorus absorption inhibitor which comprises the foregoing compound or a salt thereof.

The crosslinked polymer is hardly soluble in water, it is liable to swell through the absorption of, for instance, moisture in vivo when it is orally administered and it has a high probability of causing constipation. Contrary to this, the compound of the present invention is a non-crosslinked and water-soluble compound and therefore, it would have various advantages such that it can easily be used, that it sparingly has such a tendency of undergoing swelling in vivo when orally administered, that it sparingly causes disorders of digestive tracts and that it hardly causes, for instance, constipation.

### BEST MODE FOR CARRYING OUT THE INVENTION

In the foregoing general formulas (I) and (II), the alkyl groups included in the substituted or unsubstituted aminoalkyl group and the substituted or unsubstituted alkyl groups may be, for instance, those having 1 to 8 carbon atoms and preferably those having 1 to 3 carbon atoms. These alkyl groups may be linear or branched ones. Substituents for these groups may be, for instance, halogen atoms such as chlorine atom, hydroxyl group, cyano group and nitro group.

In the general formula (II), the aryl groups may be, for instance, a phenyl group and a naphthalene group, and preferably used herein is a phenyl group. The heteroaryl groups may be, for instance, 5-membered or 6-membered cyclic groups each containing, in the ring, an oxygen, nitrogen and/or sulfur atoms as the hetero atoms and specific examples thereof include thiophenyl group, pyridyl group, pyrimidinyl group, and pyridazinyl group. The cycloalkyl group is preferably a 4-membered to 6-membered cycloalkyl group such as a cyclopentyl group and cyclohexyl group. The alkyl groups included in the substituted or unsubstituted aminoalkyl group and the substituted or unsubstituted alkyl groups may be, for instance, those having 1 to 8 carbon atoms and preferably those having 1 to 3 carbon atoms. These alkyl groups may be linear or branched ones. Substituents for these groups may be, for instance, halogen atoms such as chlorine atom, hydroxyl group, cyano group, nitro group and amino group.

The amino-carboxylic acid residues appearing in the general formulas (I) and (II) may be, for instance, a variety of amino acid residues, various kinds of amino acid polymer residues, and guanidino-acetic acid residue. More specifically, examples thereof include residues derived from basic aliphatic amino acids such as lysine, alginine, diamino-butanoic acid and ornithine, aliphatic monoamino-monocarboxylic acids such as glycine, alanine, cyclohexyl alanine, valine and leucine; aromatic amino acids such as phenylalanine, tryptophane, and tyrosine; acidic amino acids such as aspartic acid; or residues derived from polymers of these amino acids. Among them, preferred are residues derived from basic aliphatic amino acids per se or those derived from homopolymers or copolymers thereof and particularly preferably used herein are residues derived from basic aliphatic amino acids having 5 to 8 carbon atoms (most preferably 5 or 6 carbon atoms) per se, or those derived from homopolymers or copolymers thereof. In case of the residues derived from homopolymers or copolymers, preferably used herein are residues derived from homopolymers or copolymers prepared by polymerizing 2 to 6 amino acids and in particular, 2 to 4 amino acids. The amino acid polymers may be α-type polymers obtained by the polymerization of amino groups at α-position with carboxylic acid groups or polymers other than the α-type ones such as β-, γ-, δ- or ε-type polymers obtained by the polymerization of amino acids through their amino acid groups situating at β-, γ-, δ- or ε-position with carboxylic acid groups.

Moreover, also preferably used herein include residues derived from copolymers or poly(amino acids) to which at least one acyclic amino acid residue selected from the group consisting of basic aliphatic amino acid residues, aliphatic monoamino-carboxylic acid residues and acidic amino acid residues is bonded in addition to an amino-carboxylic acid residue carrying an aryl group, a heteroaryl group or a cycloalkyl group. Examples of such copolymers are preferably those in which the amino-carboxylic acid having an aryl or heteroaryl group is phenylalanine or tryptophane; the amino-carboxylic acid having a cycloalkyl group is cyclohexyl-alanine; and the external end of the poly(amino acid) residue is an aliphatic amino acid residue other than a phenylalanine residue, a tryptophane residue or a cyclohexyl-alanine residue. Examples of such copolymer residues or poly(amino acid) residues include those which comprise a phenylalanine residue and one or at least two amino acid residues selected from ornithine, lysine, diamino-butanoic acid, valine and leucine residues bonded thereto and in which the external end of the poly(amino acid) residue is an amino acid residue other than a phenylalanine residue. Preferably used herein as such a poly(amino acid) residue are those in which the residue bonded to the nitrogen atom, X or Y, in the backbone appearing in the general formula (I) directly or through a connecting group A is a phenylalanine residue (the first amino acid residue constituting the poly(amino acid) residue), but when at least two residues selected from ornithine, lysine, diamino-butanoic acid, valine and leucine residues are linked with a phenylalanine residue, also preferably used herein are poly(amino acid) residues in which the phenylalanine residue is positioned at the interior (as the secondary or higher residue) of the corresponding poly(amino acid) residue. In this respect, the present invention is herein described while taking phenylalanine residue as an example, but the same is also true for the tryptophane residue and cyclohexyl-alanine residue.

The number of acyclic amino acid residues combined with phenylalanine, tryptophane and cyclohexyl-alanine residues preferably ranges from 1 to 5 per one residue such as a phenylalanine residue and particularly preferably 1, 2 or 3.

The foregoing amino acids may be in either of the L-form, D-form or D/L-form, but preferred is the L-form.

The connecting groups A and A1 may be —C(═O)—, —C(═O)—NH—, —NH—C(═O)—, or an alkylene group having 1 to 20 carbon atoms, which may have a substituent and whose 1 to 10 carbon atoms may be replaced with —O—, or —NRa-, —S—, —SO—, —SO₂—, and/or —C(═O)— (in the formula, Ra represents a hydrogen atom, a substituted or unsubstituted lower alkyl group, a substituted or unsubstituted acyl group, a substituted or unsubstituted alkoxy-carbonyl group, a substituted or unsubstituted carbamoyl group, or a substituted or unsubstituted sulfonyl group), and in particular, the connecting groups A and A1 each preferably represent —(CH₂)₂—NH—C(═O)—, —NH—C(═O)—, or —C(═O)—. Likewise preferably used herein are alkylene groups each having 1 to 6 carbon atoms and particularly preferably alkylene groups each having 1 to 3 carbon atoms. These alkylene groups each may have a substituent. Examples of such substituents are the same as those listed above.

Preferably used herein includes a compound wherein one of R1 and R2 appearing in the general formula (I) is a hydrogen atom and the other is a group: Rx-[A]ₚ- and one of R4 and R5 appearing therein is a hydrogen atom and the other is a group: Rx-[A]ₚ-.

In addition, preferably used herein include the foregoing compounds wherein a plurality of groups: —X(R6)- appearing in the general formula (I) represent a combination of —CH(R6)- and —N(R6)-, which may be the same or different.

Moreover, preferably used herein include the foregoing compounds wherein a plurality of groups: —Y(R7)- appearing in the general formula (I) represent a combination of —CH(R7)- and —N(R7)-, which may be the same or different.

Further, preferably used herein include the foregoing compounds wherein a plurality of groups: —X(R6)- appearing in the general formula (I) represent a combination of —CH(R6)- and —N(R6)-, which may be the same or different; and/or a plurality of groups: —Y(R7)- appearing in the general formula (I) represent a combination of —CH(R7)- and —N(R7)-, which may be the same or different; and wherein the number of the group: —N— present in the backbone of each corresponding compound thus ranges from 1 to 4.

In addition, preferably used herein include the foregoing compounds wherein a plurality of groups: —X(R6)- appearing in the general formula (I) represent groups: —CH(R6)- which may be the same or different; and a plurality of groups: —Y(R7)- appearing in the general formula (I) represent groups: —CH(R7)- which may be the same or different.

Further, preferably used herein include the foregoing compounds in which the substituent Rx appearing in the general formula (I) is an amino acid residue and p is 0.

Moreover, preferably used herein include the foregoing compounds in which all of the substituents R1 to R7 appearing in the general formula (I) do not simultaneously represent hydrogen atom.

In addition, preferably used herein include the foregoing compounds in which at least one of the substituents R1 to R7 appearing in the general formula (I) represent a group: Rx-[A]ₚ- (in the formula, the aminocarboxylic acid residue represented by Rx is a poly(amino acid) residue comprising an aminocarboxylic acid residue carrying an aryl group or a heteroaryl group and at least one amino acid residue, combined with the former, selected from the group consisting of basic aliphatic amino acid residues, aliphatic mono-aminocarboxylic acid residues and acidic amino acid residues).

Moreover, preferably used herein include the foregoing compounds in which the aminocarboxylic acid carrying an aryl group or a heteroaryl group in the general formula (I) is phenylalanine or tryptophane; and the external end of the poly(amino acid) residue is an amino acid residue other than a phenylalanine residue and a tryptophane residue.

In addition, preferably used herein include the foregoing compounds in which at least one of the substituents R1 to R7 appearing in the general formula (I) represent a group: Rx-[A]ₚ-, wherein Rx represents a group represented by Formula (II) (in the formula, B represents an aryl group, at least one of the substituents R8 to R11 represents a group: Ry-[A1]_{q}- (in the formula, A1 represents a connecting group, q represents 0 or 1, and Ry represents an aminocarboxylic acid residue or a substituted or unsubstituted aminoalkyl group)) and p represents 1.

Further, it is preferred that the water-soluble compounds represented by the general formula (I) or the salts thereof according to the present invention do not include the following compounds Dab-Am-4 and CAS No. 154487-83-9 and salts thereof:

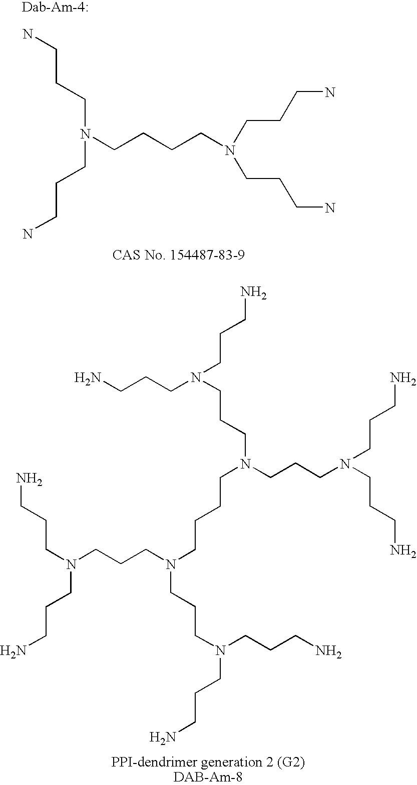

The molecular weight of the compound of the present invention represented by the foregoing general formula (I) is not restricted to any specific one, but the compound may have a molecular weight extending over a wide range on the order of 500 to 2,000,000 and preferably 500 to 3,000.

The compound of the present invention represented by the foregoing general formula (I) may be in the form of a pharmaceutically acceptable salt. Examples of such salts include inorganic salts such as hydrochlorides, hydrobromides, sulfates, nitrates and carbonates; and organic acid salts such as formates, oxalates, maleic acid salts, tartaric acid salts, citric acid salts, fumaric acid salts, acetic acid salts, propionic acid salts, butyric acid salts, benzoic acid salts, benzenesulfonic acid salts, toluenesulfonic acid salts and trifluoroacetic acid salts.

The compound of the present invention represented by the foregoing general formula (I) can easily be prepared according to, for instance, the synthetic method disclosed in Non-Patent Document 1.

For instance, the compound of the present invention is synthesized by condensing spermine (SPM), spermidine (SPD), tetraethylene-pentamine (TEPA) or pentaethylene-hexamine (PEHA) as a starting core material with, for instance, amino acids.

For instance, the compound of the present invention which comprises spermine as a core and 4 equivalents of protected amino acids condensed together can be synthesized by carrying out a condensation reaction, while using, for instance, spermine as a starting core; using, for instance dimethylformamide (DMF) as a solvent for protected amino acids; using, for instance, 2-(1H-benzotriazol-1-yl)-1,1,3,3-tetramethyl-uronium hexafluoro-phosphate (HBTU) as a condensation agent; using, for instance, N-hydroxy-benzotriazole (HOBt) as an additive; using, for instance, triethylamine (TEA) as a base. Then the Deprotection of the compound is carried out under appropriate conditions to thus give a dendrimer-shaped compound. It is also possible to synthesize the 2^{nd} generation and 3^{rd} generation dendrimer-shaped compounds by repeating a series of reactions and various dendrimer-shaped compounds can likewise be synthesized, which have substituents at different positions if variously changing protecting groups to be used.

The orally administrable phosphorus absorption inhibitor of the present invention comprises the foregoing compound. This orally administrable phosphorus absorption inhibitor is preferably in the form of a dosage form capable of being orally administered and it is particularly preferably in the form of a medical or pharmaceutical product and a food.

The orally administrable phosphorus absorption inhibitor of the present invention, in particular, permits the relaxation or alleviation of the phosphorus-intake in the patients suffering from a variety of disease caused by, for instance, the excess intake of phosphorus, such as hyper-phosphatemia, renal failure and osteoporosis, and the phosphorus absorption inhibitor is quite useful as a medicinal agent for preventing and treating these diseases.

The orally administrable phosphorus absorption inhibitor of the present invention can be prepared by processing the compound represented by the general formula (I) into a variety of forms according to the usual method. The orally administrable phosphorus absorption inhibitor of the present invention may be formed into any shape, for instance, a flat board-like shape such as a plate-like shape and a film-like shape and particulate forms and more specifically, solid forms, liquid forms, emulsions, paste-like products, jelly-like products, to thus give medical and pharmaceutical products or foods.

In other words, the orally administrable phosphorus absorption inhibitor of the present invention can efficiently be used as a food product in addition to the medical or pharmaceutical product. The orally administrable phosphorus absorption inhibitor of the present invention may be provided in the form of a product which can be ready for eating as a food without any post treatment, or a product which can be eaten after cooking, or a premixed material for preparing a food.

The foods which comprise the orally administrable phosphorus absorption inhibitor of the present invention may be in any form such as solids, powders, or granules. More specifically, the foods may be, for instance, various kinds of confectionery such as biscuits, cookies, cakes, snack foods, and rice crackers; bread, powdered beverages (such as powdered coffee, and powdered cocoa), but the food relating to the present invention is not restricted to these specific ones.

In addition, examples of foods in the form of liquids, emulsions, paste-like ones, and jelly-like ones include, but are not restricted to, a variety of beverages such as juices, carbonated beverages and lactic acid bacteria-containing beverages. Among them, preferably used herein include paste-like and jelly-like beverages.

When the orally administrable phosphorus absorption inhibitor of the present invention is, in particular, used as a medical or pharmaceutical product, the phosphorus absorption inhibitor can be formed into the usual pharmaceutical preparations in the form of, for instance, tablets, powders, granules, fine granules, and liquid preparations. These pharmaceutical preparations may be prepared according to the usual method using the phosphorus absorption inhibitor in combination with, if necessary, other pharmaceutically acceptable additives such as excipients, binders and/or lubricants.

In addition, other additives such as a sweetener and/or a coloring agent are incorporated into the pharmaceutical preparation and the resulting product may be used. The pharmaceutical preparations can be prepared according to the method disclosed in the general rules for preparations appearing in the Japanese Pharmacopoeia.

In the orally administrable phosphorus absorption inhibitor of the present invention, one should preferably intake the compound represented by the general formula (I) or a salt thereof used as an effective component of the phosphorus absorption inhibitor in an amount ranging from about 1 to about 20 g/day and preferably about 2 to 10 g/day as determined on the basis of the phosphorus-absorbing capacity thereof and accordingly, it is sufficient to set the content of the effective component to be administered three times a day immediately before meals and to design a pharmaceutical preparation such that one can intake the desired dose.

### EXAMPLES

The present invention will hereunder be described in more detail with reference to the following Examples, but the present invention is not limited to these specific Examples at all.

First of all, the abbreviations of the compounds used in the following Examples will be specified below:
TFA: trifluoro-acetyl group;
Boc: t-butoxycarbonyl group;
Z: benzyloxy-carbonyl group;
DCHA: dicyclohexyl-amine;
HBTU: 2-(1H-benzotriazol-1-yl)-1,1,3,3-tetramethyl-uronium-hexafluoro-phosphate;
HATU: (2-(7-aza-1H-benzotriazol-1-yl)-1,1,3,3-tetramethyl-uronium-hexafluorophosphates;
HCTU: (2-(6-chloro-1H-benzotriazol-1-yl)-1,1,3,3-tetramethyl-uronium-hexafluoro-phosphate;
HOBt: 1-hydroxybenzotriazole;
HOAt: 1-hydroxy-7-azabenzotriazole;
CDI: 1,1′-carbonyl-diimidazole;
TEA: triethylamine;
DIEA: diisopropyl ethylamine;
SPM: spermine;
SPD: spermidine;
DETA: diethylene-triamine;
TETA: triethylene-tetramine;
TEPA: tetraethylene-pentamine;
TAA: tris(2-aminoethyl) amine;
DMF: dimethylformamide;
GAA: guanidino-acetic acid;
Dab: diamino-butanoic acid;
Ape: 5-aminopentanoic acid;
Cha: cyclohexyl-alanine.

### Example 1

### SPM(Lys)₄ Hydrochloride

### Step 1: Condensation

DMF (19 mL) was added to a mixture of 2 g (3.78 mM) of Boc-Lys (Boc)OH.DCHA, 1.4 g (3.78 mM) of HBTU and 580 mg (3.78 mM) of HOBt.H₂O and the resulting mixture was stirred to dissolve these compounds. To the resulting solution, there were added 201 mg (0.947 mM) of spermine and 580 μl (4.16 mM) of TEA, followed by the stirring of the resulting mixture at room temperature overnight. After the completion of the reaction, ethyl acetate and a 1N aqueous solution of sodium hydroxide were added to thus extract the reaction product with the ethyl acetate. The resulting ethyl acetate phase was washed with a 0.1N aqueous solution of hydrochloric acid and then with a saturated aqueous solution of common salt, followed by the drying over sodium sulfate and the subsequent filtration. The solvent was distilled off under a reduced pressure, then the resulting residue was subjected to the reversed phase high-performance liquid chromatography using a chemically bonded octadecyl group-containing silica gel as a loading material, followed by the elution with a water/acetonitrile mixed solvent containing 0.1% (v/v) of trifluoroacetic acid and the subsequent lyophilization of the fraction containing an intended product to thus give an intermediate.

Amount of the intermediate thus obtained: 807 mg (0.53 mM) (Yield: 56%).

MS (ESI) m/z: 758 [M+2H]²⁺.

### Step 2: Deprotection

To 807 mg (0.53 mM) of the compound obtained in the foregoing step 1, there was added 3 mL of trifluoroacetic acid, and then the resulting mixture was stirred at room temperature for one hour. The solvent was distilled off under a reduced pressure, water was added to the resulting residue and the mixture was lyophilized to thus remove the excess trifluoroacetic acid.

MS (ESI) m/z: 358 [M+2H]²⁺

¹H-NMR (D₂O): δ: 1.25-1.9 (m, 32H), 2.8-3.0 (m, 8H), 3.0-3.7 (m, 12H), 3.8-3.9 (m, 2H), 4.2-4.4 (m, 2H)

### Step 3: Desalting and Conversion into Hydrochloride

After the exchange of a cation-exchange resin: Amberlite IRA910 with a 1N aqueous solution of sodium hydroxide, the ion-exchange resin was exchanged with Millipore filter-treated water till the wash liquid became neutral. Then an aqueous solution of the compound obtained in the foregoing step 2 was charged or loaded to the resin and Millipore filter-treated water was passed through the resin till the eluted liquid became neutral. The resulting aqueous solution was lyophilized, followed by the addition of hydrochloric acid to give the hydrochloride of an intended compound in a concentration of 18% by mass and the subsequent lyophilization to thus give an intended product.

Amount of the product thus obtained: 231 mg (0.32 mM) (Yield: 100%).

¹H-NMR (D₂O): δ: 1.1-1.8 (m, 32H), 2.4-2.6 (m, 8H), 3.0-3.7 (m, 16H)

### Example 2

### SPM(Lys)₁₂ Hydrochloride

### Step 1: Condensation

To a mixture comprising 3 g (5.75 mM) of Boc-Lys(Boc)OH.DCHA, 2.18 g (5.75 mM) of HBTU and 881 mg (5.75 mM) of HOBt.H₂O, there was added 20 mL of DMF, followed by the stirring of the mixture to thus dissolve the components. To the resulting solution, there were added 1.17 g (0.719 mM) of the intermediate prepared in the step 2 of Example 1 and 580 μL (4.16 mM) of TEA and the mixture was stirred at room temperature overnight. The same post-treatment used in the step 1 of Example 1 was repeated to thus give an intermediate.

MS (ESI) m/z: 1116 [M+3H]³⁺

### Step 2: Deprotection

To the compound obtained in the foregoing step 1, there was added 5 mL of trifluoroacetic acid and the resulting mixture was stirred at room temperature for one hour. The solvent was distilled off under a reduced pressure and the resulting residue was purified by the reversed phase high-performance liquid chromatography to thus give an intermediate.

Amount of the intermediate thus obtained: 1.12 g (0.21 mM) (Yield: 30%, calculated for the 2 steps).

MS (ESI) m/z: 871 [M+2H]²⁺

¹H-NMR (D₂O): δ: 1.1-1.9 (m, 80H), 2.8-3.5 (m, 32H), 3.75-3.95 (m, 8H), 4.1-4.2 (m, 4H)

### Step 3: Conversion into Hydrochloride

The compound obtained in the foregoing step 2 was subjected to the same treatments used in the step 3 of Example 1 to thus give an intended product.

¹H-NMR (D₂O): δ: 1.1-1.9 (m, 8H), 2.4-2.6 (m, 16H), 2.8-3.4 (m, 28H), 4.0-4.15 (m, 4H)

### Example 3

### SPM(Lys)₄(Arg)₈ Hydrochloride

### Step 1: Condensation

There was added 40 mL of DMF to a mixture comprising 2.4 g (7.37 mM) of Boc-Orn(TFA)OH, 2.8 g (7.37 mM) of HBTU and 1.13 g (7.37 mM) of HOBt.H₂O, followed by the stirring of them to thus dissolve the components. To the resulting solution, there were added 1.5 g (0.922 mM) of the intermediate prepared in the step 2 of Example 1 and 1.26 mL (9.03 mM) of TEA and the mixture was stirred at room temperature overnight. The same post-treatment and purification step used in the step 1 of Example 1 were repeated to thus give an intermediate.

Amount of the intermediate thus obtained: 1.17 g (0.37 mM) (Yield: 40%).

MS (ESI) m/z: 1066 [M+3H]³⁺

### Step 2: Deprotection

To 1.17 g (0.37 mM) of the compound obtained in the foregoing step 1, there was added 1.55 g (14.7 mM) of sodium carbonate, these components were dissolved in a mixed solvent comprising 8 mL of methanol and 2 mL of water, followed by the stirring of the resulting solution at room temperature overnight, the subsequent addition of 2.03 g (14.7 mM) of potassium carbonate and the additional stirring of the mixture at room temperature overnight. The insoluble matters were filtered off and the resulting filtrate was neutralized with hydrochloric acid. The solvent was distilled off under a reduced pressure and then the resulting residue was purified by the reversed phase high-performance liquid chromatography to thus give an intermediate.

Amount of the intermediate thus obtained: 312 mg (0.12 mM) (Yield: 26%).

### Step 3: Conversion into Guanigino-Derivative

There was dissolved 312 mg (0.12 mM) of the compound obtained in the step 2 in a mixed solvent comprising 7 mL of acetonitrile and 3 mL of methanol. To the resulting solution, there were added 166 mg (1.13 mM) of 1H-pyrazole-1-carboxy-amidine hydrochloride and 3134 (2.25 mM) of TEA, followed by the stirring of the resulting mixture over two nights at room temperature. The solvent was distilled off under a reduced pressure and the resulting residue was purified by the reversed phase high-performance liquid chromatography to thus give an intermediate.

Amount of the intermediate thus obtained: 228 mg (0.062 mM) (Yield: 48%).

### Step 4: Deprotection

To 228 mg (0.062 mM) of the compound obtained in the foregoing step 3, there was added 3 mL of trifluoroacetic acid, and then the resulting mixture was stirred at room temperature for one hour. The solvent was distilled off under a reduced pressure, water was added to the resulting residue and the mixture was lyophilized to thus remove the excess trifluoroacetic acid. Subsequently, the same procedures used in the step 3 of Example 1 were repeated to thus give an intended product.

Amount of the product thus obtained: 122 mg (0.062 mM) (Yield: quantitative).

MS (ESI) m/z: 983 [M+2H]²

¹H-NMR (D₂O): δ: 1.2-1.8 (m, 64H), 2.9-3.4 (m, 44H), 3.8-4.1 (m, 4H)

### Example 4

### SPM(Orn)₄ Hydrochloride

### Step 1: Condensation

There was added 30 mL of DMF to a mixture comprising 2 g (6.09 mM) of Boc-Orn(TFA)OH, 2.3 g (6.09 mM) of HATU and 829 mg (6.09 mM) of HOAt and the resulting mixture was stirred to dissolve the components. To the resulting solution, there were added 317 mg (1.52 mM) of spermine and 933 μL (6.7 mM) of TEA followed by the stirring of the mixture at room temperature overnight. After the completion of the reaction, the same post-treatment and purification procedure used in the step 1 of Example 1 were repeated to thus give an intermediate.

Amount of the intermediate thus obtained: 628 mg (0.42 mM) (Yield: 28%).

MS (ESI) m/z: 722 [M+2H]²⁺

### Step 2: Deprotection

To 644 mg (0.44 mM) of the compound obtained in the step 1, there was added 8 mL of methanol to thus completely dissolve the compound. To the resulting solution, there were added 1.9 g (17.8 mM) of sodium carbonate and 2 mL of water and the resulting mixture was stirred at 55□ overnight. After cooling the mixture to room temperature, impurities were removed through filtration, the resulting filtrate was neutralized using an aqueous hydrochloric acid solution. The solvent was distilled off under a reduced pressure and the resulting residue was purified by the reversed phase high-performance liquid chromatography to thus give an intermediate.

Amount of the intermediate thus obtained: 588 mg (0.38 mM) (Yield: 87%).

MS (ESI) m/z: 530 [M+2H]²⁺

¹H-NMR (D₂O): δ: 1.3 (s, 36H), 1.4-1.8 (m, 24H), 2.8-3.0 (m, 8H), 3.0-3.9 (m, 12H), 4.8-4.9 (m, 2H), 4.2-4.4 (m, 2H).

### Step 3: Deprotection

To 420 mg (0.27 mM) of the compound obtained in the foregoing step 2, there was added 3 mL of trifluoroacetic acid, and then the resulting mixture was stirred at room temperature for one hour. The solvent was distilled off under a reduced pressure, water was added to the resulting residue and the mixture was lyophilized to remove the excess trifluoroacetic acid and to thus give an intermediate.

### Step 4: Desalting and Conversion into Hydrochloride

The compound obtained in the foregoing step 3 was subjected to the same treatments used in the step 3 of Example 1 to thus give an intended product.

Amount of the product thus obtained: 161 mg (0.24 mM) (Yield: 88%).

MS (ESI) m/z: 330 [M+2H]²⁺

¹H-NMR (D₂O): δ: 1.2-1.8 (m, 24H), 2.4-2.6 (m, 8H), 3.0-3.7 (m, 16H).

### Example 5

### SPM(Arg)₄ Hydrochloride

### Step 1: Conversion into Guanigino-Derivative

There was dissolved 320 mg (0.21 mM) of the compound obtained in the step 2 of Example 4 in a mixed solvent comprising 2 mL of acetonitrile and 1 mL of methanol. To the resulting solution, there were added 124 mg (0.84 mM) of 1H-pyrazole-1-carboxy-amidine hydrochloride and 234 μL (1.68 mM) of TEA and the resulting mixture was stirred at room temperature overnight. The solvent was distilled off under a reduced pressure and the resulting residue was purified by the reversed phase high-performance liquid chromatography to thus give an intermediate.

Amount of the intermediate thus obtained: 213 mg (0.12 mM) (Yield: 59%).

### Step 2: Deprotection

To 213 mg (0.12 mM) of the compound obtained in the foregoing step 1, there was added 2 mL of trifluoroacetic acid, and then the resulting mixture was stirred at room temperature for one hour. The solvent was distilled off under a reduced pressure, water was added to the resulting residue and the mixture was lyophilized to thus remove the excess trifluoroacetic acid. Then the same treatments used in the step 3 of Example 1 were repeated to thus give an intended product.

Amount of the product thus obtained: 104 mg (0.12 mM) (Yield: 100%).

¹H-NMR (D₂O): δ: 1.4-1.8 (m, 24H), 3.0-3.8 (m, 24H).

### Example 6

### SPM(Lys)₆ Hydrochloride

### Step 1: Condensation

There were added 23 mL of DMF and 834 μL (6.03 mM) of TEA to a mixture comprising 1.73 g (5.06 mM) of Boc-Lys(TFA)OH, 1.92 g (5.06 mM) of HBTU, 774 mg (5.06 mM) of HOBt.monohydrate and 1.5 g (2.41 mM) of N¹,N¹²-ditrifluoroacetyl spermine.di-trifluoroacetate and the resulting mixture was stirred at room temperature overnight. The same post-treatments used in the step 1 of Example 1 were repeated to thus give an intermediate.

Yield: 430 mg

### Step 2: Deprotection

There was added 2 mL of methanol to 430 mg (0.41 mM) of the compound obtained in the foregoing step 1 to thus completely dissolve the latter. To the resulting solution, there were added 1.75 g (17.8 mM) of monosodium carbonate and 2 mL of water, followed by the stirring of the resulting mixture at room temperature overnight. After the removal of insoluble matters through filtration, the solvent was distilled off under a reduced pressure and the resulting residue was purified by repeating the same procedures used in the step 1 of Example 1 to thus give an intermediate.

Yield: 350 mg

### Step 3: Condensation

There were added 6 mL of DMF and 261 μL (1.88 mM) of TEA to a mixture comprising 663 mg (1.26 mM) of Boc-Lys(Boc)OH.DCHA, 479 mg (1.26 mM) of HATU, 171 mg (1.26 mM) of HOAt and 350 mg (0.31 mM) of the compound obtained in the foregoing step 2, followed by the stirring of the resulting mixture at room temperature overnight. The reaction solution was diluted with ethyl acetate, washed with, in order, water, a 0.1M aqueous hydrochloric acid solution, a saturated aqueous sodium bicarbonate solution and then a saturated aqueous common salt solution, and further dried over sodium sulfate. Then the solvent was distilled off and the resulting residue was purified by the same procedures used in the step 1 of Example 1 to thus give an intermediate.

### Step 4: Deprotection

To the compound obtained in the foregoing step 3, there was added 5 mL of trifluoroacetic acid, and then the resulting mixture was stirred at room temperature for one hour. The solvent was distilled off under a reduced pressure, water was added to the resulting residue and the mixture was lyophilized to thus remove the trifluoroacetic acid. Thereafter, the same procedures used in the step 3 of Example 1 were repeated to thus give an intended product (218 mg).

MS (ESI) m/z: 486.4 [M+2H]²⁺

### Example 7

### SPM(Lys)₆ Hydrochloride

### Step 1: Condensation

There were dissolved 0.755 g of Boc-Lys(Boc)OH DCHA and 0.22 g of HOBt.monohydrate in 5 mL of DMF, followed by the addition of 0.55 g of HBTU and 0.22 mL of TEA to the resulting solution and the subsequent stirring of the resulting mixture for 5 minutes. Further, there was added 2 mL of a solution of 0.33 g of di-benzyl 1,4-butan-diyl-bis[(3-aminopropyl) carbamate] in DMF, followed by the stirring thereof through the night. After subjecting the reaction solution to the post-treatment according to the usual method, it was purified by the medium pressure-silica gel column chromatography (chloroform:methanol=100/0 to 80/20) to thus give an intermediate.

Yield: 0.17 g

### Step 2: Deprotection, Desalting and Condensation

The compound obtained in the foregoing step 1 was added to 6 mL of a 1:1 mixed methylene chloride: trifluoroacetic acid solvent, the resulting mixture was stirred for one hour and the solvent was then distilled off. The resulting residue was subjected to desalting with the use of an anionic ion-exchange resin: Amberlite IRA-910.

There were dissolved, in 3 mL of DMF, the compound prepared in the preceding step, 0.84 g (1.60 mM) of Boc-Lys(Boc)OH.DCHA, 0.24 g (1.60 mM) of HOBt.monohydrate, 0.61 g (1.60 mM) of HBTU and 0.28 mL (2.00 mM) of TEA and the resulting solution was stirred through the night. After subjecting the reaction solution to the post-treatment according to the usual method, it was purified by the medium pressure-silica gel column chromatography (a gradient system of chloroform:methanol ranging from 100/0 to 80/20). To the resulting compound, there was added 5 mL of a 48% solution of HBr in acetic acid and then the mixture was stirred for one hour. After the solvent was distilled off, the resulting residue was purified by the reversed phase high-performance liquid chromatography according to the same method used in the step 1 of Example 1 to thus give a trifluoroacetate as an intended product.

Yield: 0.331 g

### Step 3: Desalting and Conversion into Hydrochloride

The compound obtained in the foregoing step 2 was subjected to the same treatments used in the step 3 of Example 1 to thus give an intended product.

Yield: 0.21 g

¹H-NMR (D₂O): δ: 4.06-4.12 (m, 2H), 3.60-3.70 (m, 8H), 2.79-3.12 (m, 40H), 1.15-1.86 (m, 80H).

MS (ESI) m/z: 870 [M+2H]²⁺

### Example 8

### SPM(Orn)₁₂ Hydrochloride

### Step 1: Condensation

There was added 10 mL of DMF to a mixture containing 1.47 g (4.44 mM) of Boc-Orn(Boc)OH, 1.68 g (4.44 mM) of HBTU and 681 mg (4.44 mM) of HOBt and then the mixture was stirred to dissolve the components. To the resulting solution, there were added 860 mg (0.55 mM) of the compound obtained in the step 3 of Example 4, and 760 μL (5.44 mM) of TEA, the resulting mixture was stirred at room temperature overnight and the same post-treatments used in the step 1 of Example 1 were repeated to thus give an intermediate.

MS (ESI) m/z: 740 [M+3H]³⁺

### Step 2: Deprotection, Desalting and Conversion into Hydrochloride

There was added 3 mL of trifluoroacetic acid to the compound prepared in the foregoing step 1 and the resulting mixture was stirred at room temperature for one hour. The solvent was distilled off under a reduced pressure, the resulting residue was purified by the reversed phase high-performance liquid chromatography and thereafter the same treatments used in the step 3 of Example 1 were repeated to thus give an intended product.

Amount of the product thus obtained: 320 mg (0.094 mM) (Overall yield for the foregoing two steps: 17%).

¹H-NMR (D₂O): δ: 1.3-1.9 (m, 64H), 2.8-3.6 (m, 28H), 3.8-4.2 (m, 12H).

### Example 9

### SPM(Orn)₄ Hydrochloride

### Step 1: Condensation

There were added 10 mL of DMF and 0.27 mL (1.92 mM) of TEA to a mixture containing 0.227 g (0.48 mM) of di-benzyl 1,4-butan-diyl-bis[(3-aminopropyl) carbamate], 573 g (1.01 mM) of Boc-Orn(TFA)Orn(TFA)OH, 382 mg (1.01 mM) of HBTU and 155 mg (1.01 mM) of HOBt and the resulting mixture was stirred at room temperature overnight. The same post-treatments used in the step 1 of Example 1 were repeated to thus give an intermediate.

### Step 2: Deprotection

To the compound obtained in the foregoing step 1, there was added 30 mL of methanol and the compound was completely dissolved in the solvent. To the resulting solution, there were added 816 mg (7.68 mM) of sodium carbonate and 6 mL of water and the resulting mixture was stirred at room temperature overnight. The insoluble matters were filtered off, the solvent was then distilled off under a reduced pressure, and then the resulting residue was subjected to the same post-treatments used in the step 1 of Example 1 to thus give an intermediate.

### Step 3: Deprotection

There were added, to the compound obtained in the foregoing step 2, 30 mL of methanol and a catalytic amount of 10% Pd/C and the resulting mixture was stirred in a hydrogen gas atmosphere all the night through. The catalyst was filtered off, the solvent was then distilled off and the resulting residue was purified by the same procedures used in the step 1 of Example 1 to thus give an intermediate.

### Step 4: Deprotection and Desalting

Trifluoroacetic acid (3 mL) was added to the compound prepared in the foregoing step 3 and the resulting mixture was stirred at room temperature for one hour. The solvent was removed through distillation under a reduced pressure, followed by the addition of water to the resulting residue and the lyophilization of the aqueous mixture to thus remove the excess trifluoro-acetic acid. Then, the same treatments used in the step 3 of Example 1 were repeated to thus give the intended product (148 mg).

### Example 10

### SPM(Lys)₂(Orn)₂ Hydrochloride

### Step 1: Condensation

There were dissolved, in 5 mL of methylene chloride, 1.06 g (2.00 mM) of Boc-Lys(Boc)OH DCHA and 0.32 g (2.00 mM) of CDI and the resulting mixture was stirred at room temperature for one hour. To this solution, there was added 0.20 g (1.00 mM) of spermine and the mixture was stirred at room temperature through the night. Then the solvent was distilled off, the resulting residue was purified by the reversed phase high-performance liquid chromatography, followed by the lyophilization of the purified fraction and the desalting of the resulting compound using an anion-exchange resin: Amberlite IRA-910.

Amount of the compound obtained: 0.32 g (0.37 mM)

### Step 2: Condensation

There were dissolved, in 2 mL of DMF, 0.25 g (0.75 mM) of Boc-Orn (Boc)OH, 0.28 g (0.75 mM) of HATU and 0.10 g (0.75 mM) of HOAt, and then 0.13 mL (0.93 mM) of TEA was added to the resulting solution. After stirring the mixture for 5 minutes, 0.32 g (0.37 mM) of the compound prepared in the foregoing step 1 was added to the mixture followed by the stirring thereof through the night. The solvent was then distilled off, the resulting residue was purified by the reversed phase high-performance liquid chromatography to thus recover the fraction containing an intended product and the fraction was lyophilized to thus give an intermediate.

Amount of the intermediate obtained: 0.20 g

### Step 3: Deprotection

The compound prepared in the foregoing step 2 (0.20 g) was dissolved in 5 mL of a methylene chloride: trifluoroacetic acid mixed solvent and the solution was stirred for one hour. The solvent was distilled off and the resulting residue was then purified by the reversed phase high-performance liquid chromatography to thus give an intermediate.

Yield: Quantitative

### Step 4: Desalting and Conversion into Hydrochloride

The compound prepared in the foregoing step 3 was subjected to the same treatments used in the step 3 of Example 1 to thus give an intended product.

Yield: 101 mg

¹H-NMR (D₂O): δ: 1.20-1.90 (m, 28H), 2.78-3.95 (m, 20H), 4.00-4.30 (m, 2H)

MS (ESI) m/z: 687 [M+H]⁺

### Example 11

### SPM(Lys)₈ Hydrochloride

### Step 1: Condensation

There were added 30 mL of DMF and 1.67 mL (12.1 mM) of TEA to a mixture containing 5.3 g (10.1 mM) of Boc-Lys(Boc)OH.DCHA, 3.84 g (10.1 mM) of HATU, 1.37 g (10.1 mM) of HOAt and 2.0 g (4.82 mM) of N1,N12-ditrifluoroacetyl-spermine.di-trifluoroacetate and the resulting mixture was stirred at room temperature overnight. The same post-treatments used in the step 1 of Example 1 were repeated to thus give an intermediate.

### Step 2: Deprotection

Trifluoroacetic acid (10 mL) was added to the compound prepared in the foregoing step 1 and the mixture was stirred at room temperature for one hour. The solvent was distilled off under a reduced pressure, followed by the addition of water and the subsequent lyophilization to thus remove the excess trifluoroacetic acid.

### Step 3: Condensation

There were added 15 mL of DMF and 9524, (6.88 mM) of TEA to a mixture containing the compound prepared in the foregoing step 2, 1.73 g (3.28 mM) of Boc-Lys(Boc)OH.DCHA, 1.25 g (3.28 mM) of HATU and 446 mg (3.28 mM) of HOAt and the resulting mixture was stirred at room temperature overnight. The same post-treatments used in the step 1 of Example 1 were repeated to thus give an intermediate.

Yield: 840 mg

### Step 4: Deprotection

Methanol (4 mL) was added to the compound 840 mg (0.72 mM) prepared in the foregoing step 3 to thus completely dissolve the latter in the methanol. To the resulting solution, there were added 3.0 g (28.9 mM) of sodium carbonate and 4 mL of water and the resulting mixture was stirred at room temperature overnight. After the removal of the insoluble matters through filtration, the solvent was distilled off under a reduced pressure, the resulting residue was subjected to the reversed phase high-performance liquid chromatography whose column was packed with a chemically bonded octadodecyl group-containing silica gel and the column was eluted with water:acetonitrile mixed solvent containing 0.1% (v/v) of trifluoroacetic acid and the fractions containing an intended product was lyophilized to thus give the title compound.

Yield: 700 mg

### Step 5: Condensation

There were added 8 mL of DMF and 94 μL (0.68 mM) of TEA to a mixture containing the compound (400 mg) prepared in the foregoing step 4, 238 mg (0.45 mM) of Boc-Lys(Boc)OH.DCHA, 172 mg (0.45 mM) of HATU and 61 mg (0.45 mM) of HOAt and the resulting mixture was stirred at room temperature overnight. The same post-treatments used in the step 1 of Example 1 were repeated to thus give an intermediate.

Yield: 385 mg

### Step 6: Deprotection

Trifluoroacetic acid (5 mL) was added to the compound prepared in the foregoing step 5 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid.

### Step 7: Desalting and Conversion into Hydrochloride

The compound prepared in the foregoing step 6 was subjected to the same treatments used in the step 3 of Example 1 to thus give an intended product.

Yield: 133 mg

MS (ESI) m/z: 614.6 [M+2H]²⁺

### Example 12

### SPM(Lys)₆ Hydrochloride

### Step 1: Deprotection

Trifluoroacetic acid (5 mL) was added to the compound (300 mg) prepared in the step 4 of Example 11 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid.

Yield: Quantitative

### Step 2: Desalting and Conversion into Hydrochloride

The compound prepared in the foregoing step 1 was subjected to the same treatments used in the step 3 of Example 1 to thus give an intended product.

Yield: 124 mg

MS (ESI) m/z: 486.4 [M+2H]²⁺

### Example 13

### SPM(Orn)₂(Lys)₂ Hydrochloride

The same procedures used in the step 1 of Example 10 were repeated except for using Boc-Orn(Boc)OH in place of the Boc-Lys(Boc)OH DCHA used therein and the same procedures used in the step 2 of Example 10 were repeated except for using Boc-Lys(Boc)OH DCHA instead of the Boc-Orn(Boc)OH used therein to thus give an intended product.

Yield: 308 mg

¹H-NMR (D₂O): δ: 1.25-1.90 (m, 28H), 2.79-3.90 (m, 20H), 4.15-4.30 (m, 2H)

MS (ESI) m/z: 687 [M+H]⁺

### Example 14

### SPM(Orn)₈ Hydrochloride

### Step 1: Condensation

There was added 12 mL of DMF to a mixture containing 1.17 g (3.53 mM) of Boc-Orn(Boc)OH, 1.34 g (3.53 mM) of HATU, 480 mg (3.53 mM) of HOAt and the resulting mixture was stirred for the dissolution of the components. To the resulting solution, there were added 935 mg (0.88 mM) of a compound prepared by repeating the same procedures used in the step 2 of Example 4 and 541 μL (3.88 mM) of TEA and the resulting mixture was stirred at room temperature overnight. After the completion of the reaction, the same post-treatments and purification step used in the step 1 of Example 1 were repeated to thus give an intermediate.

Amount of the intermediate obtained: 758 mg (0.32 mM) (Yield: 37%)

### Step 2: Deprotection, Desalting and Conversion into Hydrochloride

Trifluoroacetic acid (5 mL) was added to 800 mg (0.34 mM) of the compound prepared in the foregoing step 1 and the resulting mixture was stirred at room temperature for one hour. The solvent was distilled off under a reduced pressure, followed by the addition of water to the resulting residue and the lyophilization of the aqueous mixture to thus remove the excess trifluoro-acetic acid. The same treatments used in the step 3 of Example 1 were repeated to thus give an intended product.

Amount of the product obtained: 346 mg (0.31 mM) (Yield: 89%)

¹H-NMR (D₂O): δ: 1.2-1.8 (m, 40H), 2.4-2.6 (m, 8H), 2.9-3.4 (m, 26H), 3.5-3.8 (m, 2H)

### Example 15

### SPM(Lys)₈ Hydrochloride

### Step 1: Condensation

DMF (30 mL) and TEA (8.82 mL, 8.82 mM) were added to a mixture containing 13.9 g (40.6 mM) of Boc-Lys(TFA)OH, 15.4 g (40.6 mM) of HBTU, 6.2 g (40.6 mM) of HOBt and 2 g (9.90 mM) of spermine and the resulting mixture was stirred at room temperature overnight. After the completion of the reaction, the same post-treatments and purification step used in the step 1 of Example 1 were repeated to thus give an intermediate.

Yield: 1.6 g

### Step 2: Deprotection

Methanol (5 mL) was added to 850 mg (0.57 mM) of the compound prepared in the foregoing step 1 and the latter was completely dissolved in the methanol. To the resulting solution, there were added 2.4 g (22.7 mM) of sodium carbonate and 5 mL of water and the resulting mixture was stirred at room temperature overnight. After the removal of the insoluble matters through filtration, the solvent was distilled off under a reduced pressure and the resulting residue was subjected to the same purification step used in the step 1 of Example 1 to thus give an intermediate.

Yield: 800 mg

### Step 3: Condensation

DMF (15 mL) and TEA (595 μL, 4.3 mM) were added to a mixture containing the compound prepared in the foregoing step 2, 982 mg (2.87 mM) of Boc-Lys(TFA)OH, 1.09 g (2.87 mM) of HATU and 390 mg (2.87 mM) of HOAt and the resulting mixture was stirred at room temperature overnight. After the completion of the reaction, the same post-treatments and purification step used in the step 1 of Example 1 were repeated to thus give an intermediate.

Yield: 920 mg

### Step 4: Deprotection

Methanol (50 mL) was added to 920 mg of the compound prepared in the foregoing step 3 and the latter was completely dissolved in the methanol. To the resulting solution, there were added 10 g of sodium carbonate and 40 mL of water, followed by the stirring of the resulting mixture at room temperature overnight. After the removal of the insoluble matters through filtration, the solvent was distilled off under a reduced pressure and the resulting residue was subjected to the same purification step used in the step 1 of Example 1 to thus give an intermediate.

Yield: 880 mg

### Step 5: Deprotection

Trifluoroacetic acid (5 mL) was added to the compound (300 mg) prepared in the foregoing step 4 and the resulting mixture was stirred at room temperature for one hour. The solvent was distilled off under a reduced pressure, followed by the addition of water to the resulting residue and the lyophilization of the aqueous mixture to thus remove the excess trifluoro-acetic acid.

Yield: Quantitative

### Step 6: Desalting and Conversion into Hydrochloride

The compound prepared in the foregoing step 5 was subjected to the same treatments used in the step 3 of Example 1 to thus give an intended product.

Yield: 135 mg

MS (ESI) m/z: 614.6 [M+2H]²⁺

### Example 16

### SPM(Lys)₈ Hydrochloride

### Step 1: Deprotection

Trifluoroacetic acid (10 mL) was added to the compound (750 mg) prepared in the step 1 of Example 15 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid.

Yield: Quantitative

### Step 2: Condensation

DMF (15 mL) and TEA (415 μL, 3.0 mM) were added to a mixture containing the compound obtained in the foregoing step 1, 1.06 g (2.0 mM) of Boc-Lys(Boc)OH.DCHA, 760 mg (2.0 mM) of HATU, and 272 mg (2.0 mM) of HOAt and the resulting mixture was stirred at room temperature overnight. After the completion of the reaction, the same post-treatments and purification step used in the step 1 of Example 1 were repeated to thus give an intermediate.

Yield: 970 mg

### Step 3: Deprotection

Methanol (70 mL) was added to 920 mg of the compound obtained in the foregoing step 2 and the compound was completely dissolved in the methanol. To the resulting solution, there were added 10 g of sodium carbonate and 40 mL of water and the resulting mixture was stirred at room temperature overnight. After the removal of the insoluble matters through filtration, the solvent was distilled off under a reduced pressure and the resulting residue was subjected to the same purification step used in the step 1 of Example 1 to thus give an intermediate.

### Step 4: Deprotection

Trifluoroacetic acid (5 mL) was added to the compound prepared in the foregoing step 3 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid.

Yield: Quantitative

### Step 5: Desalting and Conversion into Hydrochloride

The compound prepared in the foregoing step 4 was subjected to the same treatments used in the step 3 of Example 1 to thus give an intended product.

Yield: 330 mg

MS (ESI) m/z: 614.6 [M+2H]²⁺

### Example 17

### SPM(Orn)₂ Hydrochloride

### Step 1: Condensation

There were dissolved, in 8 mL of dichloromethane, 997 mg (3.0 mM) of Boc-Orn(Boc)OH and 486 mg (3.0 mM) of CDI and the resulting mixture was stirred at room temperature for one hour. To the resulting solution, there was added 303 mg (1.5 mM) of spermine and the resulting mixture was stirred at room temperature overnight. The solvent was distilled off under a reduced pressure and the resulting residue was purified by the reversed phase high-performance liquid chromatography to thus give an intermediate.

Amount of the intermediate obtained: 1.14 g (1.3 mM) (Yield: 87%)

MS (ESI) m/z: 831 [M+H]⁺

### Step 2: Deprotection, Desalting and Conversion into Hydrochloride

Trifluoroacetic acid (3 mL) was added to 370 mg (0.35 mM) of the compound prepared in the foregoing step 1 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same treatments used in the step 3 of Example 1 were repeated to thus give an intended product.

Amount of the product obtained: 150 mg (0.35 mM) (Yield: Quantitative)

MS (ESI) m/z: 431 [M+H]⁺

### Example 18

### SPM(Arg)₂ Hydrochloride

### Step 1: Condensation

There was stirred 1.41 g (4.00 mM) of Z-Arg(NO2)-OH and 0.65 g (4.00 mM) of CDI in a mixed solvent containing 10 mL of methylene chloride and 3 mL of DMF for one hour, and 0.40 g (2 mM) of spermine was added to the resulting mixture, followed by the stirring of the mixture at room temperature overnight. The solvent was distilled off and the resulting residue was purified by the same method used in the step 1 of Example 1 to thus give an intermediate.

Yield: 0.38 g (0.41 mM)

### Step 2: Deprotection

To the compound prepared in the foregoing step 1, there were added 10 mL of methanol, 50 μL of concentrated hydrochloric acid and a catalytic amount of 10% Pd/C and the resulting mixture was stirred under a hydrogen gas atmosphere all the night through. After the catalyst was once removed through filtration, the reaction system was again prepared followed by the stirring thereof through the night. After the removal of the catalyst through filtration, the solvent was distilled off and the resulting residue was purified by the same method used in the step 1 of Example 1 to thus give an intermediate.

### Step 3: Desalting and Conversion into Hydrochloride

The compound prepared in the foregoing step 2 was subjected to the same treatments used in the step 3 of Example 1 to thus give an intended product.

Yield: 111 mg

¹H-NMR (D₂O): δ: 3.87-3.92 (m, 2H), 2.98-3.26 (m, 16H), 1.42-1.88 (m, 16H)

MS (ESI) m/z: 259 [M+2H]²⁺

### Example 19

### SPM(Arg)₂(Lys)₂ Hydrochloride

### Step 1: Condensation

There were dissolved, in 100 mL of dichloromethane, 4.84 g (14.8 mM) of Boc-Arg(TFA)OH and 2.39 g (14.8 mM) of CDI and the resulting solution was stirred at room temperature for one hour. Spermine (1.49 g, 7.4 mM) was added to the resulting solution and the mixture was stirred overnight. After the completion of the reaction, the same purification step used in the step 1 of Example 1 was repeated to thus give an intermediate.

Amount of the intermediate obtained: 3.7 g (3.53 mM) (Yield: 48%)

### Step 2: Condensation

DMF (12 mL) was added to a mixture containing 1.05 g (2.0 mM) of Boc-Lys(Boc)OH.DCHA, 760 mg (2.0 mM) of HATU and 272 mg (2.0 mM) of HOAt to thus give a solution, there were then added, to this solution, 1.05 g (1.0 mM) of the compound prepared in the foregoing step 1 and 306 μL (2.2 mM) of TEA and the resulting mixture was stirred at room temperature overnight. After the completion of the reaction, the same post-treatments and purification step used in the step 1 of Example 1 were repeated to thus give an intermediate.

Amount of the intermediate obtained: 488 mg (0.33 mM) (Yield: 33%)

MS (ESI) m/z: 740 [M+2H]²⁺

### Step 3: Deprotection

Methanol (8 mL) was added to 488 mg (0.33 mM) of the compound prepared in the foregoing step 2 and the latter was completely dissolved in the methanol. To this solution, there were added 1.39 g (13.2 mM) of sodium carbonate and 2 mL of water and the mixture was stirred at 55□ overnight. After cooling the mixture to room temperature, the insoluble matters were removed through filtration and the resulting filtrate was neutralized by the addition of an aqueous solution of hydrochloric acid. The solvent was distilled off under a reduced pressure, the resulting residue was subjected to the same purification treatments used in the step 1 of Example 1 to thus give an intermediate.

Amount of the intermediate obtained: 321 mg (0.24 mM) (Yield: 75%)

MS (ESI) m/z: 644 [M+2H]²⁺

### Step 4: Conversion into Guanidino Derivative

The compound (321 mg, 0.24 mM) prepared in the foregoing step 3 was completely dissolved in 3 mL of methanol. To this solution, there were added 73 mg (0.5 mM) of 1H-pyrazole-1-carboxy-amidine hydrochloride and 139 μL (1.0 mM) of TEA and the resulting mixture was stirred at room temperature overnight. The solvent was distilled off under a reduced pressure, the resulting residue was subjected to the same purification treatments used in the step 1 of Example 1 to thus give an intermediate.

Amount of the intermediate obtained: 296 mg (0.17 mM) (Yield: 74%)

MS (ESI) m/z: 686 [M+2H]²⁺

### Step 5: Deprotection, Desalting and Conversion into Hydrochloride

Trifluoroacetic acid (3 mL) was added to the compound (296 mg, 0.17 mM) prepared in the foregoing step 4 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same procedures used in the step 3 of Example 1 were repeated to thus give an intended product.

Amount of the product obtained: 131 mg (0.17 mM) (Yield: 100%)

¹H-NMR (D₂O): δ: 1.0-1.8 (m, 28H), 2.4-2.55 (M, 4H), 2.8-3.7 (m, H), 3.8-3.9 (m, 20H)

### Example 20

### SPM(Lys)₁₂ Hydrochloride

### Step 1: Condensation

There were added 50 mL of DMF and 415 μL (5.92 mM) of TEA to a mixture containing the compound (2 g) prepared in the step 4 of Example 15, 1.35 g (3.94 mM) of Boc-Lys(TFA)OH, 1.5 g (3.94 mM) of HATU and 536 mg (3.94 mM) of HOAt and the resulting mixture was stirred at room temperature overnight. After the completion of the reaction, the same post-treatments and purification step used in the step 1 of Example 1 were repeated to thus give an intermediate.

Yield: 475 mg

### Step 2: Deprotection

There was added 10 mL of trifluoroacetic acid to the compound obtained in the foregoing step 1 and the resulting mixture was stirred at room temperature for one hour. The solvent was distilled off under a reduced pressure, followed by the addition of water to the resulting residue and the lyophilization of the aqueous mixture to thus remove the excess trifluoro-acetic acid.

Yield: Quantitative

### Step 3: Condensation

There were added 10 mL of DMF and 1644 (1.19 mM) of TEA to a mixture containing the compound prepared in the foregoing step 2, 271 mg (0.79 mM) of Boc-Lys(TFA)OH, 302 mg (0.79 mM) of HATU and 108 mg (0.79 mM) of HOAt and the resulting mixture was stirred at room temperature overnight. After the completion of the reaction, the same post-treatments and purification step used in the step 1 of Example 1 were repeated to thus give an intermediate.

Yield: 500 mg

### Step 4: Deprotection

Methanol (30 mL) was added to 500 mg of the compound prepared in the foregoing step 3 and the latter was completely dissolved in the methanol. To this solution, there were added 10 g of sodium carbonate and 20 mL of water and the mixture was stirred at room temperature overnight. After the removal of the insoluble matters through filtration, the solvent was distilled off under a reduced pressure and the resulting residue was subjected to the same purification step used in the step 1 of Example 1 to thus give an intermediate.

Yield: 380 mg

### Step 5

Trifluoroacetic acid (3 mL) was added to the compound prepared in the foregoing Deprotection step 4 and the resulting mixture was stirred at room temperature for one hour. The solvent was distilled off under a reduced pressure, followed by the addition of water to the resulting residue and the lyophilization of the aqueous mixture to thus remove the excess trifluoro-acetic acid.

Yield: Quantitative

### Step 6: Desalting and Conversion into Hydrochloride

The compound prepared in the foregoing step 5 was subjected to the same treatments used in the step 3 of Example 1 to thus give an intended product.

Yield: 202 mg

MS (ESI) m/z: 580.9 [M+3H]³⁺, 436.0 [M+4H]⁴⁺

### Example 21

### SPM(Gly)₄ Hydrochloride

### Step 1: Condensation

There were added 30 mL of DMF and 0.89 mL (6.37 mM) of TEA to a mixture containing 2.2 g (5.79 mM) of Boc-Gly-OH, 2.2 g (5.79 mM) of HBTU and 293 mg (1.45 mM) of spermine and the resulting mixture was stirred at room temperature overnight. After the completion of the reaction, the same post-treatments and purification step used in the step 1 of Example 1 were repeated to thus give an intermediate.

Amount of the intermediate obtained: 285 mg (0.34 mM) (Yield: 24%)

### Step 2: Deprotection, Desalting and Conversion into Hydrochloride

Trifluoroacetic acid (3 mL) was added to the compound prepared in the foregoing step 1 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same procedures used in the step 3 of Example 1 were repeated to thus give an intended product.

Amount of the product obtained: 132 mg (0.30 mM) (Yield: 89%)

¹H-NMR (D₂O): δ: 1.35-1.55 (m, 4H), 1.55-1.8 (m, 4H), 3.0-3.5 (m, 20H)

### Example 22

### SPM(Orn)₈ Hydrochloride

### Step 1: Condensation

There were added 80 mL of DMF and 2.5 mL (2.50 mM) of TEA to a mixture containing 6.38 g (11.9 mM) of Boc-Orn(TFA)Orn(TFA)OH, 4.5 g (11.9 mM) of HATU, 1.6 g (11.9 mM) of HOAt and 600 mg (2.96 mM) of spermine and the resulting mixture was stirred at room temperature overnight. After the completion of the reaction, the same post-treatments and purification step used in the step 1 of Example 1 were repeated to thus give an intermediate.

Yield: 2.57 g

### Step 2: Deprotection

There was added 90 mL of methanol to the compound prepared in the foregoing step 1 to thus completely dissolve the latter in the methanol. To the resulting solution, there were added 10 g of sodium carbonate and 40 mL of water and the resulting mixture was stirred at room temperature overnight. After the removal of the insoluble matters through filtration, the solvent was distilled off under a reduced pressure and the resulting residue was subjected to the same purification step used in the step 1 of Example 1 to thus give an intermediate.

Yield: 1.65 g

### Step 3: Deprotection

Trifluoroacetic acid (5 mL) was added to the compound prepared in the foregoing step 2 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid.

Yield: Quantitative

### Step 4: Desalting and Conversion into Hydrochloride

The compound prepared in the foregoing step 3 was subjected to the same treatments used in the step 3 of Example 1 to thus give an intended product.

Yield: 875 mg

MS (ESI) m/z: 558.4 [M+2H]²⁺, 372.6 [M+3H]³⁺, 279.8 [M+4H]⁴⁺

### Example 23

### SPM(Lys)₂(Arg)₂ Hydrochloride

### Step 1: Condensation

There were dissolved 2.11 g (4.0 mM) of Boc-Lys(Boc)OH.DCHA and 648 mg (4.0 mM) of CDI in 10 mL of dichloromethane and the resulting mixture was stirred at room temperature for one hour. To the resulting solution, there was added 417 mg (2.0 mM) of spermine and this mixture was stirred at room temperature overnight. The solvent was distilled off under a reduced pressure and the resulting residue was purified by the reversed phase high-performance liquid chromatography to thus give an intermediate.

Amount of the product obtained: 910 mg (0.84 mM) (Yield: 42%)

¹H-NMR (D₂O): δ: 1.2-1.8 (m, H), 2.9-3.0 (t, H), 3.8-4.0 (m, H)

### Step 2: Condensation

DMF (5 mL) was added to a mixture containing 328 mg (1.0 mM) of Boc-Orn(TFA)OH, 380 mg (1.0 mM) of HATU and 136 mg (1.0 mM) of HOAt and the resulting mixture was stirred to dissolve the components. To the resulting solution, there were added 430 mg (0.5 mM) of the compound synthesized in the foregoing step 1 and 1534 (1.1 mM) of TEA and the resulting mixture was stirred at room temperature overnight. Then, the same post-treatments and purification step used in the step 1 of Example 1 were repeated to thus give an intermediate.

Amount of the intermediate obtained: 154 mg (0.1 mM) (Yield: 20%)

MS (ESI) m/z: 740 [M+2H]²⁺

### Step 3: Deprotection

There was dissolved 514 mg (0.34 mM) of the compound obtained in the foregoing step 2 in a mixed solvent comprising 12 mL of methanol and 3 mL of water, there was added 1.47 g (13.8 mM) of sodium carbonate to the resulting solution and then the mixture was stirred at 55□ overnight. After cooling the mixture to room temperature, the insoluble matters were removed through filtration and the filtrate was neutralized by the addition of an aqueous solution of hydrochloric acid. The solvent was distilled off under a reduced pressure and the resulting residue was subjected to the same post-treatment and purification step used in the step 1 of Example 1 to thus give an intermediate.

Amount of the intermediate obtained: 349 mg (0.26 mM) (Yield: 78%)

MS (ESI) m/z: 644 [M+2H]²⁺

### Step 4: Conversion into Guanidino Derivative

There was dissolved 349 mg (0.26 mM) of the compound obtained in the foregoing step 3 in a mixed solvent comprising 4 mL of acetonitrile and 1 mL of methanol. To this solution, there were added 77 mg (0.53 mM) of 1H-pyrazole-1-carboxy-amidine hydrochloride and 147 μL (1.06 mM) of TEA and the resulting mixture was stirred at room temperature overnight. The solvent was distilled off under a reduced pressure and the resulting residue was purified by the reversed phase high-performance liquid chromatography to thus give an intermediate.

Amount of the intermediate obtained: 288 mg (0.17 mM) (Yield: 69%)

MS (ESI) m/z: 686 [M+2H]²⁺

### Step 5: Deprotection, Desalting and Conversion into Hydrochloride

Trifluoroacetic acid (3 mL) was added to 288 mg (0.17 mM) of the compound prepared in the foregoing step 4 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same procedures used in the step 3 of Example 1 were repeated to thus give an intended product.

Amount of the product obtained: 151 mg (0.19 mM) (Yield: 93%)

¹H-NMR (D₂O): δ: 1.0-1.8 (m, 28H), 2.35-2.5 (m, 4H), 2.8-3.45 (m, 20H), 3.5-3.7 (m, 2H)

### Example 24

### SPM(Arg)₂ Hydrochloride

### Step 1: Protection

There was dissolved 1.04 g (5 mM) of spermine in 5 mL of DMF, then 20674 (11 mM) of t-butyl-phenyl carbonate was added to the resulting solution and the mixture was stirred at room temperature overnight. After the completion of the reaction, an aqueous solution of hydrochloric acid was added to the reaction solution which had been cooled to 0□ using an ice bath to thus adjust the pH thereof to 3. After the insoluble matters were extracted with dichloromethane, the pH value of the reaction system was controlled to 10 with an aqueous solution of sodium hydroxide and then the intended product was back-extracted with dichloromethane. The extract was dried over sodium sulfate, followed by the removal of the sodium sulfate through filtration and the resulting filtrate was subjected to distillation under a reduced pressure to thus give the title compound.

Amount of the title compound obtained: 1.98 g (4.9 mM) (Yield: 98%)

MS (ESI) m/z: 403 [M+H]⁺

### Step 2: Synthesis of SPM(Boc)₂[Boc-Orn(TFA)]₂

DMF (15 mL) was added to a mixture containing 656 mg (2.0 mM) of Boc-Orn(TFA)OH, 760 mg (2.0 mM) of HATU and 272 mg (2.0 mM) of HOAt and the resulting mixture was stirred to thus dissolve the components in DMF. To this solution, there were added 402 mg (1.0 mM) of the compound obtained in the foregoing step 1 and 306 μL (2.2 mM) of TEA and the resulting mixture was stirred at room temperature overnight. After the completion of the reaction, the same post-treatments and purification step used in the step 1 of Example 1 were repeated to thus give the title compound.

Amount of the title compound obtained: 416 mg (0.4 mM) (Yield: 40%)

MS (ESI) m/z: 1024 [M+H]⁺

### Step 3: Deprotection

There was completely dissolved 546 mg (0.53 mM) of the compound obtained in the foregoing step 2 in 12 mL of methanol. To the resulting solution, there were added 2.26 g (21.3 mM) of sodium carbonate and 3 mL of water and the resulting mixture was stirred at 55□ overnight. After the completion of the reaction, the insoluble matters were removed through filtration and the resulting filtrate was neutralized using an aqueous solution of hydrochloric acid. The solvent was distilled off under a reduced pressure and the resulting residue was purified by repeating the same procedures used in the step 1 of Example 1 to thus give the title compound.

Amount of the title compound obtained: 330 mg (0.3 mM) (Yield: 58%)

MS (ESI) m/z: 832 [M+H]⁺

### Step 4: Conversion into Guanidino Derivative

There was dissolved 330 mg (0.3 mM) of the compound obtained in the foregoing step 3 in a mixed solvent containing 3 mL of acetonitrile and 1 mL of methanol, then there were added, to the resulting solution, 91 mg (0.62 mM) of 1H-pyrazole-1-carboxy-amidine hydrochloride and 173 μL (1.24 mM) of TEA and the resulting mixture was stirred at room temperature overnight. The solvent was distilled off under a reduced pressure and the resulting residue was purified by repeating the same procedures used in the step 1 of Example 1 to thus give the title compound.

Amount of the title compound obtained: 276 mg (0.24 mM) (Yield: 80%)

MS (ESI) m/z: 458 [M+2H]²⁺

### Step 5: Deprotection, Desalting and Conversion into Hydrochloride

Trifluoroacetic acid (4 mL) was added to 276 mg (0.24 mM) of the compound prepared in the foregoing step 4 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same procedures used in the step 3 of Example 1 were repeated to thus give an intended product.

Amount of the product obtained: 117 mg (0.24 mM) (Yield: Quantitative)

¹H-NMR (D₂O): δ: 1.3-1.95 (m, 14H), 2.7-3.5 (m, 16H), 3.6-3.8 (m, 2H)

### Example 25

### SPM(GAA)₄ Hydrochloride

### Step 1: Conversion into Guanidino Derivative, Desalting and Conversion into Hydrochloride

There was dissolved 824 mg (0.93 mM) of a TFA salt of the compound obtained in the step 2 of Example 21 in a mixed solvent comprising 5 mL of acetonitrile and 5 mL of methanol. Then, there were added, to the resulting solution, 550 mg (3.75 mM) of 1H-pyrazole-1-carboxy-amidine hydrochloride and 1.04 mL (7.51 mM) of TEA and the resulting mixture was stirred at room temperature overnight. The solvent was distilled off under a reduced pressure and the resulting residue was purified by the reversed phase high-performance liquid chromatography and the resulting compound was subjected to the same treatments used in the step 3 of Example 1 to thus give an intended product.

Amount of the product obtained: 632 mg (0.59 mM) (Yield: 64%)

MS (ESI) m/z: 300 [M+2H]²⁺

¹H-NMR (D₂O): δ: 1.35-1.55 (m, 4H), 1.55-1.8 (m, 4H), 3.05-3.3 (m, 12H), 3.87 (s, 4H), 4.05 (s, 4H)

### Example 26

### SPM(Ape)₄ Hydrochloride

### Step 1: Condensation

DMF (30 mL) and TEA (0.89 mL, 6.37 mM) were added to a mixture comprising 1.26 g (5.79 mM) of Boc-ApeOH, 2.2 g (5.79 mM) of HBTU and 293 mg (1.45 mM) of spermine and the resulting mixture was stirred at room temperature overnight. After the completion of the reaction, the same post-treatments and purification step used in the step 1 of Example 1 were repeated to thus give an intermediate.

Amount of the intermediate obtained: 666 mg (0.34 mM) (Yield: 46%)

### Step 2: Deprotection, Desalting and Conversion into Hydrochloride

Trifluoroacetic acid (3 mL) was added to 666 mg (0.34 mM) of the compound prepared in the foregoing step 1 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same procedures used in the step 3 of Example 1 were repeated to thus give an intended product.

Amount of the product obtained: 159 mg (0.20 mM) 59%)

### Example 27

### SPM(Lys)₉ Hydrochloride

### Step 1: Synthesis of N-{3-[4-(3-Aminopropylamino)-butylamino]-propyl}-2,2,2-trifluoroacetamide

Trifluoroacetic acid (12 mL) was added to a solution of 2.7 g (4.52 mM) of t-butyl ester of (3-t-butoxycarbonylamino-propyl)-(4-{t-butoxycarbonyl-[3-(2,2,2-trifluoroacetylamino)-propyl]-amino}butyl)-carbamic acid in dichloro-methane (12 mL) and the mixture was stirred at room temperature for 20 hours. The solvent was distilled off under a reduced pressure, followed by the addition of water to the resulting residue and the lyophilization of the aqueous mixture to thus give the title compound.

Yield: Quantitative

¹H-NMR (DMSO): δ: 1.62 (4H, m), 1.86 (4H, m), 2.91 (10H, m), 3.29 (2H, q, J=6.7 Hz), 7.95-9.60 (5H, m)

MS (ESI) m/z: 299 [M+H]⁺

### Step 2: BocLys(Boc)Lys[BocLys(Boc)]OMe

DMF (50 mL) was added to a mixture comprising 18.12 g of BocLys(Boc)OH DCHA and 5.24 g of HOBt monohydrate to thus dissolve the latter in DMF. To the resulting solution, there were added 13.00 g of HBTU, 7.2 mL of TEA and 4.00 g of lysine methyl ester di-hydrochloride and the resulting mixture was stirred at room temperature through the night. After the solvent was distilled off, the resulting residue was post-treated according to the usual method and the residue was purified by the medium pressure silica gel column chromatography (CHCl₃:MeOH=99:1 to 85:15) to thus give an intended product.

Yield: 7.70 g

### Step 3: BocLys(Boc)Lys[BocLys(Boc)]OH

The compound obtained in the foregoing step 1 was dissolved in 30 mL of tetrahydrofuran, 15 mL of methanol and 15 mL of water, then 0.51 g of lithium hydroxide monohydrate was added to the resulting mixture and the mixture was stirred at room temperature for 2 hours. After about half of the solvent was distilled off, the mixture was post-treated according to the usual method and the solvent was distilled off to thus give an intended product.

Yield: 6.78 g

¹H-NMR (CDCl₃): δ: 1.12-2.13 (m, 54H), 2.85-3.25 (m, 6H), 4.10-5.05 (m, 3H), 6.20-6.50 (m, 3H), 7.35-7.85 (m, 3H)

MS (ESI) m/z: 803 [M+H]⁺

### Step 4: Condensation

TEA (3.74 mL, 26.8 mM) was added to a solution of 1.71 g (2.68 mM) of the compound obtained in the foregoing step 1, 6.46 g (8.04 mM) of the compound obtained in the foregoing step 3, 3.06 g (8.04 mM) of HATU and 1.09 g (8.04 mM) of HOAt in 50 mL of DMF and the resulting mixture was stirred at room temperature for 24 hours. After the completion of the reaction, the same post-treatments and purification step used in the step 1 of Example 1 were repeated to thus give an intermediate.

Amount of the product obtained: 3.14 g (1.18 mM) (Yield: 44%)

MS (ESI) m/z: 885 [M+3H]³⁺

### Step 5: Deprotection

Sodium carbonate (3 g) was added to a solution of 3.12 g (1.18 mM) of the compound obtained in the foregoing step 4 in 20 mL of water and 50 mL of methanol and the resulting mixture was stirred at room temperature for 4 hours. The insoluble matters were removed through filtration while washing with methanol and the methanol was distilled off under a reduced pressure. After the completion of the reaction, the same post-treatments and purification step used in the step 1 of Example 1 were repeated to thus give an intermediate.

Amount of the intermediate obtained: 1.65 g (0.65 mM) (Yield: 55%)

MS (ESI) m/z: 853 [M+3H]³⁺

### Step 6: Deprotection, Desalting and Conversion into Hydrochloride

Trifluoroacetic acid (5 mL) was added to a solution of 601 mg (0.225 mM) of the compound obtained in the foregoing step 5 in 5 mL of dichloromethane and the mixture was stirred at room temperature for 2 hours. After the solvent was distilled off under a reduced pressure, the resulting residue was subjected to the same purification step used in the step 1 of Example 1 and it was then subjected to the same treatments used in the step 3 of Example 1 to thus give an intended product.

Amount of the product obtained: 246 mg (0.184 mM) (Yield: 82%)

¹H-NMR (D₂O): δ: 1.12-1.80 (62H, m), 2.40-2.90 (14H, m), 2.95-3.40 (22H, m), 3.75-4.20 (3H, m)

MS (ESI) m/z: 453 [M+3H]³⁺

### Example 28

### SPM(Orn)₁₂ Hydrochloride

### Step 1: BocOrn(TFA)Orn(TFA)Orn(TFA)OBn

A solution of Isobutyl chloroformate (1.12 mL, 9.32 mM) in THF (6 mL) was dropwise added to a mixture comprising 2.91 g (8.88 mM) of BocOrn(TFA)OH, 1.02 mL (9.32 mM) of N-methyl-morpholine, 6 mL of THF and 60 mL of DMF, while cooling the mixture to −40□. After stirring the mixture at that temperature for one hour, there was added, to the stirred mixture, a solution of H-Orn(TFA)Orn(TFA)OBn.hydrochloride (5 g, 8.88 mM) and N-methyl-morpholine (1.95 mL, 17.8 mM) in DMF (20 mL) and the resulting mixture was stirred at room temperature overnight. The reaction solution was diluted with ethyl acetate, washed with, in order, water, a 0.1M aqueous hydrochloric acid solution, a saturated sodium bicarbonate solution, and a saturated aqueous solution of common salt, then dried over sodium sulfate, and then the solvent was distilled off to thus give the title compound.

Yield: 7.0 g

### Step 2: BocOrn(TFA)Orn(TFA)Orn(TFA)OH

To the compound obtained in the foregoing step 1, there were added 1.5 g of 10% Pd/C (moisture content: 50%) and 100 mL of methanol and the resulting mixture was stirred at room temperature overnight, in a hydrogen gas atmosphere. The reaction liquid was filtered through cerite and then the solvent was distilled off to thus give the title compound.

Yield: 6.2 g

### Step 3: Condensation

DMF (50 mL) and TEA (2.5 mL, 14.4 mM) were added to a mixture comprising 6.2 g (8.29 mM) of Boc-Orn(TFA)Orn(TFA)Orn(TFA)OH, 3.15 g (8.29 mM) of HATU, 1.13 g (8.29 mM) of HOAt and 419 mg (2.07 mM) of spermine, and the mixture was stirred at room temperature overnight. The reaction solution was diluted with ethyl acetate, washed with, in order, water, a 0.1M aqueous hydrochloric acid solution, a saturated sodium bicarbonate solution, and a saturated aqueous solution of common salt, then dried over sodium sulfate, and then the solvent was distilled off and the resulting residue was subjected to the same post-treatment and purification step used in the step 1 of Example 1 to thus give and intermediate.

Yield: 900 mg

### Step 4: Deprotection

Methanol (80 mL) was added to the compound obtained in the foregoing step 3 to completely dissolve the latter in the methanol. To the resulting solution, there were added 10 g of sodium carbonate and 40 mL of water and this mixture was stirred at room temperature overnight. After the insoluble matters were filtered off, the solvent was distilled off under a reduced pressure and the resulting residue was purified by the same procedures used in the step 1 of Example 1 to thus give an intermediate.

Yield: 573 mg

### Step 5: Deprotection, Desalting and Conversion into Hydrochloride

Trifluoroacetic acid (10 mL) was added to the compound obtained in the foregoing step 4 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same treatments used in the step 3 of Example 1 were repeated to thus give an intended product.

Yield: 260 mg

MS (ESI) m/z: 524.8 [M+3H]⁺, 393.8 [M+4H]⁴⁺

### Example 29

### SPM(Lys)₁₂ Hydrochloride

### Step 1: Condensation

DMF (60 mL) and TEA (1.13 mL, 8.16 mM) were added to a mixture comprising 2.75 g of the compound obtained in the step 4 of Example 15, 1.86 g (5.44 mM) of Boc-Lys(TFA)OH, 2.07 g (5.44 mM) of HATU and 740 mg (5.44 mM) of HOAt and the resulting mixture was stirred at room temperature overnight. After the completion of the reaction, the same post-treatments and purification step used in the step 1 of Example 1 were repeated to thus give an intermediate.

Yield: 2.5 g

### Step 2: Deprotection

Methanol (80 mL) was added to 2.5 g of the compound obtained in the foregoing step 1 and the latter was completely dissolved in the methanol. To the resulting solution, there were added 10 g of sodium carbonate and 40 mL of water and this mixture was stirred at room temperature overnight. After the insoluble matters were filtered off, the solvent was distilled off under a reduced pressure and the resulting residue was purified by the same procedures used in the step 1 of Example 1 to thus give an intermediate.

Yield: 1.92 g

### Step 3: Deprotection

Trifluoroacetic acid (10 mL) was added to the compound obtained in the foregoing step 2 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid.

Yield: Quantitative

### Step 4: Desalting and Conversion into Hydrochloride

The compound prepared in the foregoing step 3 was subjected to the same treatments used in the step 3 of Example 1 to thus give an intended product.

Yield: 940 mg

MS (ESI) m/z: 580.9 [M+3H]³⁺, 436.0 [M+4H]⁴⁺

### Example 30

### SPM(Orn)₂(Arg)₂ Hydrochloride

### Step 1: Condensation

DMF (11 mL) was added to a mixture containing 908 mg (2.76 mM) of Boc-Orn(TFA)OH, 1.05 g (2.76 mM) of HATU and 376 mg (2.76 mM) of HOAt, followed by the stirring of the resulting mixture to dissolve the components. To this solution, there were added 1.14 g (1.3 mM) of the compound obtained in the step 1 of Example 17 and 424 μL (3.04 mM) of TEA, followed by the stirring of the resulting mixture at room temperature overnight. The same post-treatments and purification step used in the step 1 of Example 1 to thus give an intermediate.

Amount of the intermediate obtained: 829 mg (0.56 mM) (Yield: 41%)

MS (ESI) m/z: 726 [M+2H]²⁺

### Step 2: Deprotection

The compound (829 mg, 0.57 mM) obtained in the foregoing step 1 was dissolved in a mixed solvent comprising 10 mL of methanol and 2.5 mL of water, then 2.4 g (22.8 mM) of sodium carbonate was added to the resulting solution and the mixture was stirred at 55□ overnight. After cooling the mixture to room temperature, the insoluble matters were filtered off, and the resulting filtrate was neutralized by the addition of an aqueous solution of hydrochloric acid. The solvent was distilled off under a reduced pressure and the resulting residue was purified by the reversed phase high-performance liquid chromatography to thus give an intermediate.

Amount of the intermediate obtained: 659 mg (0.43 mM) (Yield: 77%)

MS (ESI) m/z: 630 [M+2H]²⁺

### Step 3: Conversion into Guanidino Derivative

The compound (878 mg, 0.59 mM) obtained in the foregoing step 2 was dissolved in a mixed solvent comprising 4 mL of acetonitrile and 1 mL of methanol. To this solution, there were added 173 mg (1.18 mM) of 1H-pyrazole-1-carboxy-amidine hydrochloride and 329 μL (2.36 mM) of TEA and the resulting mixture was stirred at room temperature overnight. The solvent was distilled off under a reduced pressure and the resulting residue was purified by the reversed phase high-performance liquid chromatography to thus give an intermediate.

Amount of the intermediate obtained: 848 mg (0.53 mM) (Yield: 91%)

MS (ESI) m/z: 672 [M+2H]²⁺

### Step 4: Deprotection, Desalting and Conversion into Hydrochloride

Trifluoroacetic acid (5 mL) was added to 848 mg (0.53 mM) of the compound obtained in the foregoing step 3 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same treatments used in the step 3 of Example 1 were repeated to thus give an intended product.

Amount of the product obtained: 357 mg (0.47 mM) (Yield: 90%)

¹H-NMR (D₂O): δ: 1.2-1.8 (m, 24H), 2.4-2.5 (m, 4H), 2.85-3.5 (m, 18H), 3.6-3.75 (m, 2H)

### Example 31

### SPD(Orn)₃ Hydrochloride

### Step 1: Condensation

The procedures of the step 1 of Example 1 were repeated except that Boc-Orn(TFA)OH was substituted for the Boc-Lys(Boc)OH.DCHA and that spermidine was substituted for the spermine, to thus give an intermediate.

### Step 2: Deprotection

The compound (800 mg, 0.74 mM) obtained in the foregoing step 1 was dissolved in a mixed solvent comprising 9 mL of methanol and 3 mL of water, then 3.1 g (29.7 mM) of sodium carbonate was added to the resulting solution and the mixture was stirred at 55□ overnight. After the completion of the reaction, the insoluble matters were filtered off and the resulting filtrate was neutralized using an aqueous hydrochloric acid solution. The solvent was distilled off under a reduced pressure and the resulting residue was purified by the reversed high-performance liquid chromatography to thus give an intended product.

Amount of the product obtained: 507 mg (0.44 mM) (Yield: 60%)

MS (ESI): 789 [M+H]⁺

### Step 3: Deprotection and Desalting

Trifluoroacetic acid (3 mL) was added to 200 mg (0.17 mM) of the compound obtained in the foregoing step 2 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same treatments used in the step 3 of Example 1 were repeated to thus give an intended product.

Amount of the product obtained: 86 mg (0.17 mM) (Yield: Quantitative)

MS (ESI): 489 [M+H]⁺

### Example 32

### SPD(Arg)₃ Hydrochloride

### Step 1: Conversion into Guanidino Derivative

The compound (300 mg, 0.26 mM) obtained in the step 2 of Example 31 was dissolved in a mixed solvent comprising 1 mL of acetonitrile and 4 mL of methanol, then 116 mg (0.79 mM) of 1H-pyrazole-1-carboxy-amidine hydrochloride and 2224 (1.59 mM) of TEA were added to the resulting solution and the mixture was stirred at room temperature overnight. The solvent was distilled off under a reduced pressure, the resulting residue was purified by the reversed high-performance liquid chromatography and then lyophilized to thus give an intended product.

Amount of the product obtained: 215 mg (0.16 mM) (Yield: 63%)

MS (ESI): 472 [M+2H]²⁺

### Step 2: Deprotection, Desalting and Conversion into Hydrochloride

Trifluoroacetic acid (3 mL) was added to 215 mg (0.16 mM) of the compound obtained in the foregoing step 1 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same procedures used in the step 3 of Example 1 were repeated to thus give an intended product.

Amount of the product obtained: 112 mg (0.15 mM) (Yield: 91%)

¹H-NMR (D₂O): δ: 1.3-1.8 (m, 18H), 2.8-3.95 (m, 17H)

### Example 33

### SPD(Orn)₂ Hydrochloride

### Step 1: Condensation

There were dissolved, in 150 mL of dichloromethane, 9.95 g (6 mM) of Boc-Orn(Boc)OH and 4.86 g (6 mM) of CDI and the resulting solution was stirred at room temperature for one hour. To this solution, there was added 2.35 mL (3 mM) of spermidine and the mixture was stirred at room temperature overnight. The solvent was distilled off under a reduced pressure and the resulting residue was purified by the reversed phase high-performance liquid chromatography to thus give an intermediate.

Amount of the intermediate obtained: 5.2 g (10.7 mM) (Yield: 71%)

### Step 2: Deprotection, Desalting and Conversion into Hydrochloride

Trifluoroacetic acid (3 mL) was added to 250 mg (0.28 mM) of the compound obtained in the foregoing step 1 and the resulting mixture was stirred at room temperature for one hour. The solvent was distilled off under a reduced pressure, followed by the addition of water to the resulting residue and the lyophilization of the aqueous mixture to thus remove the excess trifluoroacetic acid. Then the same procedures used in the step 3 of Example 1 were repeated to thus give an intended product.

Amount of the product obtained: 124 mg (0.28 mM) (Yield: Quantitative)

¹H-NMR (D₂O): δ: 1.4-1.85 (m, 14H), 2.8-3.4 (m, 12H), 3.8-3.9 (m, 2H)

### Example 34

### TETA(Orn)₂[Orn(Orn)₂]₂ TFA Salt

### Step 1: Condensation

There were dissolved, in 8 mL of dichloromethane, 1.6 g (4.00 mM) of Z-Orn(Z)OH and 648 mg (4.00 mM) of CDI and the resulting solution was stirred at room temperature for one hour. To this solution, there was added 298 μL (2.00 mM) of triethylene tetramine and the resulting mixture was stirred through the night. The solvent was distilled off and the resulting residue was purified by the reversed phase high-performance liquid chromatography and then the fraction containing an intended product was lyophilized to thus give the intermediate for an intended compound.

Amount of the intermediate obtained: 1.29 g (1.12 mM) (Yield: 56%)

MS (ESI): m/z: 911 [M+H]⁺

### Step 2: Condensation

There were dissolved, in 8 mL of DMF, 832 mg (2.5 mM) of Boc-Orn(Boc)OH, 949 mg (2.5 mM) of HATU and 340 mg (2.5 mM) of HOAt. To this solution, there were added 1.29 g (1.12 mM) of the compound obtained in the foregoing step 1 and 1.03 mL (7.3 mM) of TEA and the resulting mixture was stirred through the night. After the completion of the reaction, the same treatments used in the step 1 of Example 1 were repeated to thus give an intermediate.

Amount of the intermediate obtained: 1.15 g (0.72 mM) (Yield: 65%)

MS (ESI): m/z: 770 [M+2H]²⁺

### Step 3: Deprotection

There was dissolved, in trifluoroacetic acid (4 mL), 1.15 g (0.72 mM) of the compound obtained in the foregoing step 2 and the resulting solution was stirred for one hour. The solvent was distilled off, followed by the addition of water to the resulting residue for the dissolution of the compound and the lyophilization of the aqueous solution to thus give an intermediate.

Yield: Quantitative

### Step 4: Condensation

There was dissolved, in 10 mL of DMF, 1.1 g (3.28 mM) of Boc-Orn(Boc)OH, 1.25 g (3.28 mM) of HATU and 448 mg (3.28 mM) of HOAt. To this solution, there were added 820 mg (0.72 mM) of the compound obtained in the foregoing step 3 and 1.35 mL (9.71 mM) of TEA and the resulting mixture was stirred at room temperature through the night. After the completion of the reaction, the same treatments used in the step 1 of Example 1 were repeated to thus give an intermediate.

Amount of the intermediate obtained: 1.45 g (0.60 mM) (Yield: 81%)

MS (ESI): m/z: 1198 [M+2H]²⁺

### Step 5: Deprotection

To 1.45 g (0.60 mM) of the compound obtained in the foregoing step 4, there was added 145 mg of 10% Pd/C and then the mixture was dissolved and dispersed in 8 mL of methanol. The atmosphere of the reaction system was replaced with hydrogen gas and then the system was stirred at room temperature overnight. After the Pd/C was filtered off, the solvent was distilled off, the resulting residue was purified by the reversed phase high-performance liquid chromatography and the fraction containing an intended product was lyophilized to thus give an intermediate.

Amount of the intermediate obtained: 1.04 g (0.45 mM) (Yield: 75%)

MS (ESI): m/z: 930 [M+2H]²⁺

### Step 6: Deprotection

The compound (1.04 g, 0.45 mM) obtained in the foregoing step 5 was dissolved in 5 mL of trifluoroacetic acid and the solution was stirred for one hour. The solvent was distilled off, the resulting residue was purified by the reversed phase high-performance liquid chromatography and the fraction containing an intended product was lyophilized to thus give the intended product.

Amount of the product obtained: 768 mg (0.31 mM) (Yield: 70%)

MS (ESI): m/z: 530 [M+2H]²⁺

¹H-NMR (D₂O): δ: 1.4-1.95 (m, 32H), 2.85-4.0 (m, 36H)

### Example 35

### TETA[Orn(Dab)₂]₄ TFA Salt

### Step 1: Condensation

There were dissolved, in 200 mL of dichloromethane, 15.35 g (41.9 mM) of Z-Orn(Boc)OH and 6.8 g (41.9 mM) of CDI and the resulting solution was stirred at room temperature for one hour. To this solution, there was added 3.13 mL (21 mM) of triethylene tetramine and the mixture was stirred at room temperature through the night. After the completion of the reaction, the same post-treatment used in the step 1 of Example 1 was repeated.

There were dissolved, in 30 mL of DMF, 4.5 g (12.3 mM) of Z-Orn(Boc)OH, 4.7 g (12.3 mM) of HATU and 1.67 g (12.3 mM) of HOAt. To the resulting solution, there were added 4.7 g (5.58 mM) of the compound obtained in step 3 and 3.3 mL (23.7 mM) of TEA and the resulting mixture was stirred at room temperature through the night. After the completion of the reaction, the same post-treatment used in the step 1 of Example 1 was repeated, 10% Pd/C was added to the resulting residue and the resulting mixture was dissolved and dispersed in 100 mL of methanol. After the atmosphere of the solution was replaced with hydrogen, the solution was stirred at room temperature overnight. After the Pd/C was filtered off, the solvent was distilled off, the resulting residue was purified by the reversed phase high-performance liquid chromatography and the fraction containing an intended product was lyophilized to thus give an intermediate.

Amount of the intermediate obtained: 2.76 g (2.75 mM)

### Step 2: Deprotection

There was dissolved, in 4 mL of trifluoroacetic acid, 730 mg (0.5 mM) of the compound obtained in the foregoing step 1 and the resulting solution was stirred for one hour. The solvent was distilled off, followed by the addition of water to the resulting residue to thus dissolve the compound and the lyophilization of the aqueous solution to thus give an intermediate.

### Step 3: Condensation

There were dissolved, in 12 mL of DMF, 2.1 g (4.4 mM) of Boc-Dab(Boc)OH.DCHA, 1.66 g (4.4 mM) of HATU and 598 mg (4.4 mM) of HOAt. To this solution, there were added 820 g (0.72 mM) of the compound obtained in the foregoing step 2 and 1.88 mL (13.5 mM) of TEA and the resulting mixture was stirred through the night. After the completion of the reaction, the same treatment used in the step 1 of Example 1 was repeated to thus give an intermediate.

Amount of the intermediate obtained: 992 mg (0.22 mM) (Yield: 44% calculated for the two steps)

MS (ESI): m/z: 1002 [M+3H]³⁺

### Step 4: Deprotection

There was dissolved, in 4 mL of trifluoroacetic acid, 992 mg (0.22 mM) of the compound obtained in the foregoing step 3 and the resulting solution was stirred for one hour. The solvent was distilled off and the resulting residue was purified by the reversed phase high-performance liquid chromatography and the fraction containing an intended product was lyophilized to thus give an intermediate.

Amount of the intermediate obtained: 699 mg (0.21 mM) (Yield: 98%)

MS (ESI): m/z: 468 [M+3H]³⁺

¹H-NMR (D₂O): δ: 1.4-1.7 (m, 16H), 2.05-2.25 (m, 16H), 2.95-3.8 (m, 36H), 3.8-4.3 (m, 12H)

### Example 36

### TETA[Orn(Orn)₂]₂(Orn)₂ TFA Salt

### Step 1: Condensation

There were dissolved, in 8 mL of dichloromethane, 1.33 g (4.00 mM) of Boc-Orn(Boc)OH, and 648 mg (4.00 mM) of CDI and the resulting solution was stirred at room temperature for one hour. To this solution, there was added 298 μL (2.00 mM) of triethylene tetramine and the mixture was stirred at room temperature through the night. The solvent was distilled off and the resulting residue was purified by the reversed phase high-performance liquid chromatography and the fraction containing an intended product was lyophilized to thus give an intermediate.

Amount of the intermediate obtained: 1.31 g (1.3 mM) (Yield: 65%)

MS (ESI): m/z: 775 [M+H]⁺

### Step 2: Condensation

There were dissolved, in 10 mL of DMF, 1.15 g (2.89 mM) of Z-Orn(Z)OH, 1.1 g (2.89 mM) of HATU and 393 mg (2.89 mM) of HOAt. To this solution, there were added 1.31 g (1.3 mM) of the compound obtained in the foregoing step 1 and 1.19 mL (8.54 mM) of TEA and the resulting mixture was stirred through the night. After the completion of the reaction, the same treatments used in the step 1 of Example 1 were repeated to thus give an intermediate.

Amount of the intermediate obtained: 1.35 g (0.87 mM) (Yield: 67%)

MS (ESI): m/z: 770 [M+2H]²⁺

### Step 3: Deprotection

There was dissolved, in 4 mL of trifluoroacetic acid, 1.35 g (0.87 mM) of the compound obtained in the foregoing step 2 and the resulting solution was stirred for one hour. The solvent was distilled off, followed by the addition of water to the resulting residue to thus dissolve the compound and the lyophilization of the aqueous solution to thus give an intermediate.

### Step 4: Condensation

There were dissolved, in 10 mL of DMF, 1.27 g (3.83 mM) of Boc-Orn(Boc)OH, 1.45 g (3.83 mM) of HATU and 521 mg (3.83 mM) of HOAt. To this solution, there were added 991 mg (0.87 mM) of the compound obtained in the foregoing step 3 and 1.57 mL (11.3 mM) of TEA and the resulting mixture was stirred at room temperature through the night. After the completion of the reaction, the same procedures used in the step 1 of Example 1 were repeated to thus give an intermediate.

Amount of the intermediate obtained: 1.63 g (0.67 mM) (Yield: 78% calculated for the two steps)

MS (ESI): m/z: 1198 [M+2H]²⁺

### Step 5: Deprotection

There was added 163 mg of 10% Pd/C to 1.63 g (0.67 mM) of the compound obtained in the foregoing step 4 and then the resulting mixture was dissolved and dispersed in 8 mL of methanol. After replacing the atmosphere of the reaction system with hydrogen, the system was stirred at room temperature overnight. After the Pd/C was filtered off, the solvent was distilled off, the resulting residue was purified by the reversed phase high-performance liquid chromatography and the fraction containing an intended product was lyophilized to thus give an intermediate.

Amount of the intermediate obtained: 1.15 g (0.50 mM) (Yield: 75%)

MS (ESI): m/z: 930 [M+2H]²⁺

### Step 6: Deprotection

There was dissolved, in 5 mL of trifluoroacetic acid, 1.15 g (0.50 mM) of the compound prepared in the foregoing step 5 and the solution was stirred for one hour. The solvent was distilled off and the resulting residue was purified by the reversed phase high-performance liquid chromatography and the fraction containing an intended product was lyophilized to thus give the intended product.

Amount of the product obtained: 1.27 g (0.52 mM) (Yield: 87%)

MS (ESI): m/z: 530 [M+2H]²⁺

¹H-NMR (D₂O): δ: 1.375-1.95 (m, 32H), 2.8-3.0 (m, 12H), 3.0-3.7 (m, 16H), 3.8-3.9 (t, 2H), 3.9-4.0 (t, 2H), 4.05-4.2 (m, 2H), 4.35-4.45 (m, 2H)

### Example 37

### TETA(PheOrn)₂(OrnOrn)₂ Hydrochloride

### Step 1: Condensation

There were dissolved, in 20 mL of dichloromethane, 0.97 g (6.0 mM) of CDI and 1.79 g (6.0 mM) of Z-L-Phe-OH and the solution was stirred at room temperature for 30 minutes. Triethylene tetramine (0.447 mL, 3.0 mM) was added to the resulting solution and the mixture was stirred at room temperature through the night. The solvent was distilled off and the resulting residue was purified by the reversed phase high-performance liquid chromatography and the fraction containing an intended product was lyophilized to thus give an intermediate.

Amount of the intermediate obtained: 2.1 g (2.24 mM, 75%)

### Step 2: Condensation

There were dissolved, in 15 mL of DMF, 1.64 g (4.48 mM) of Z-Orn(Boc)-OH, 0.67 g (4.48 mM) of HOAt and 1.87 g (4.48 mM) of HATU, followed by the addition of 1.25 mL of TEA and the subsequent stirring of the resulting mixture for 10 minutes. To the resulting mixture, there was added the compound (2.1 g) obtained in the foregoing step 1 and the mixture was stirred through the night. After the completion of the reaction, the same procedures used in the step 1 of Example 1 were repeated to thus give an intermediate.

Yield: 1.50 g

### Step 3: Deprotection and Condensation

The compound obtained in the foregoing step 2 was dissolved in 30 mL of methanol, and the compound was hydrogenated within a hydrogen gas atmosphere in the presence of a catalytic amount of 10% Pd/C. After confirming the completion of the reaction, the Pd/C was filtered off and then the solvent was distilled off to thus give a residue. There were dissolved, in 10 mL of DMF, 1.53 g (4.61 mM) of Boc-Orn(Boc)-OH, 0.69 g (4.61 mM) of HOAt and 1.93 g (4.61 mM) of HATU, then 0.8 mL of TEA was added to the resulting solution and the mixture was stirred for 10 minutes. The foregoing residue was added to the mixture and the latter was stirred at room temperature through the night. After the completion of the reaction, the same procedures used in the step 1 of Example 1 were repeated to thus give an intermediate.

### Step 4: Deprotection

The compound obtained in the foregoing step 3 was dissolved in 20 mL of trifluoroacetic acid and the solution was stirred for 2 hours. The solvent was distilled off and the resulting residue was purified by the reversed phase high-performance liquid chromatography and the fraction containing an intended product was lyophilized to thus give an intermediate.

### Step 5: Ion-Exchanging and Conversion into Hydrochloride

The same procedures used in the step 3 of Example 1 were repeated to thus give an intended compound.

Yield: 0.82 g

¹H-NMR (D₂O): δ: 7.15-7.41 (m, 10H), 4.44-4.62 (m, H), 3.88-4.13 (m, 4H), 2.63-3.77 (m, 28H), 1.54-2.02 (m, 24H)

MS (ESI): m/z: 1112 [M+H]⁺

### Example 38

### TETA(Orn)₂[Orn(Orn)₂]₂ Hydrochloride

### Step 1: Ion-Exchanging and Conversion into Hydrochloride

The compound obtained in Example 34 was treated by the same procedures used in the step 3 of Example 1 to thus give an intended compound.

Yield: 339 mg (0.27 mM)

¹H-NMR (D₂O): δ: 1.4-1.8 (m, 32H), 2.8-3.0 (m, 12H), 3.0-4.0 (m, 24H)

### Example 39

### TETA[Orn(Orn)₂]₂(Orn)₂ Hydrochloride

### Step 1: Ion-Exchanging and Conversion into Hydrochloride

The compound obtained in Example 36 was treated by the same procedures used in the step 3 of Example 1 to thus give an intended compound.

Yield: 614 mg (0.49 mM)

¹H-NMR (D₂O): δ: 1.35-1.8 (m, 32H), 2.8-3.0 (m, 12H), 3.0-3.7 (m, 20H), 3.7-4.0 (m, 2H), 4.1-4.3 (m, 2H)

### Example 40

### TETA(Phe-δOrnOrn)₄ Hydrochloride

### Step 1: Condensation

There were dissolved, in 15 mL of DMF, 2.6 g (8.8 mM) of Z-Phe-OH, 3.3 g (8.8 mM) of HATU and 1.2 g (8.8 mM) of HOAt. To the resulting solution, there were added 298 μL (2.0 mM) of triethylene tetramine and 3.0 mL (22.0 mM) of TEA and the resulting mixture was stirred at room temperature through the night. After the completion of the reaction, the same procedures used in the step 1 of Example 1 were repeated to thus give an intermediate.

Amount of the intermediate obtained: 1.07 g (0.84 mM) (Yield: 42%)

MS (ESI): m/z: 636 [M+2H]²⁺

### Step 2: Deprotection

There was added 107 mg of 10% Pd/C to 1.07 g (0.84 mM) of the compound obtained in the foregoing step 1 and then the resulting mixture was dissolved and dispersed in 15 mL of methanol. After replacing the atmosphere of the reaction system with hydrogen, the reaction system was stirred at room temperature overnight. After the removal of the Pd/C through filtration, the solvent was distilled off, the resulting residue was purified by the reversed phase high-performance liquid chromatography and the fraction containing an intended product was lyophilized to thus give an intermediate.

Amount of the intermediate obtained: 757 mg (0.58 mM) (Yield: 70%)

MS (ESI): m/z: 735 [M+H]⁺

### Step 3: Condensation

There were dissolved, in 10 mL of DMF, 1.20 g (2.79 mM) of Boc-Orn(Z)OH, 1.06 g (2.79 mM) of HATU and 381 mg (2.79 mM) of HOAt. To this solution, there were added 757 mg (0.58 mM) of the compound obtained in the foregoing step 2 and 975 μL (6.99 mM) of TEA and the resulting mixture was stirred at room temperature through the night. After the completion of the reaction, the same operations used in the step 1 of Example 1 were repeated to thus give an intermediate.

Amount of the intermediate obtained: 1.13 g (0.48 mM) (Yield: 84%)

MS (ESI): m/z: 1064 [M+2H]²⁺

### Step 4: Deprotection

There was added 113 mg of 10% Pd/C to 1.13 g (0.48 mM) of the compound obtained in the foregoing step 3 and the resulting mixture was dissolved and dispersed in 6 mL of methanol. After replacing the atmosphere of the reaction system with hydrogen, the reaction system was stirred at room temperature overnight. After the removal of the Pd/C through filtration, the solvent was distilled off, the resulting residue was purified by the reversed phase high-performance liquid chromatography and the fraction containing an intended product was lyophilized to thus give an intermediate.

Amount of the intermediate obtained: 841 mg (0.37 mM) (Yield: 77%)

MS (ESI): m/z: 796 [M+H]⁺

### Step 5: Condensation

There were dissolved, in 8 mL of DMF, 663 mg (1.8 mM) of Boc-Orn(Z)OH, 689 mg (1.8 mM) of HATU and 246 mg (1.8 mM) of HOAt. To this solution, there were added 841 mg (0.37 mM) of the compound obtained in the foregoing step 4 and 630 μL (4.52 mM) of TEA and the resulting mixture was stirred at room temperature through the night. After the completion of the reaction, the same operations used in the step 1 of Example 1 were repeated to thus give an intermediate.

Amount of the intermediate obtained: 1.01 g (0.31 mM) (Yield: 84%)

MS (ESI): m/z: 996 [M+3H]³⁺

### Step 6: Deprotection

There was added 101 mg of 10% Pd/C to 1.01 g (0.31 mM) of the compound obtained in the foregoing step 5 and the resulting mixture was dissolved and dispersed in 10 mL of methanol. After replacing the atmosphere of the reaction system with hydrogen, the reaction system was stirred at room temperature overnight. After the removal of the Pd/C through filtration, the solvent was distilled off, the resulting residue was purified by the reversed phase high-performance liquid chromatography and the fraction containing an intended product was lyophilized to thus give an intermediate.

Amount of the intermediate obtained: 679 mg (0.21 mM) (Yield: 68%)

MS (ESI): m/z: 817 [M+3H]³⁺

### Step 7: Deprotection

There was dissolved, in 4 mL of trifluoroacetic acid, 679 mg (0.21 mM) of the compound obtained in the foregoing step 6 and the solution was stirred for one hour. The solvent was distilled off, the resulting residue was purified by the reversed phase high-performance liquid chromatography and the fraction containing an intended product was lyophilized to thus give an intermediate.

Amount of the intermediate obtained: 585 mg (0.19 mM) (Yield: 92%)

MS (ESI): m/z: 824 [M+2H]²⁺

### Step 8: Ion-Exchanging and Conversion into Hydrochloride

The compound (585 mg, 0.19 mM) obtained in the foregoing step 7 was subjected to the same operations used in the step 3 of Example 1 to thus give an intended compound.

Amount of the product obtained: 376 mg (0.18 mM)

¹H-NMR (D₂O): δ: 1.3-1.85 (m, 32H), 2.6-3.25 (m, 28H), 3.6-3.95 (m, 8H), 7.0-7.4 (m, 20H)

### Example 41

### TETA[Orn(Dab)₂]₄ Hydrochloride

### Step 1: Ion-Exchanging and Conversion into Hydrochloride

The compound obtained in Example 35 was subjected to the same operations used in the step 3 of Example 1 to thus give an intended compound.

Amount of the compound obtained: 301 mg (0.18 mM) (Yield: 98%)

¹H-NMR (D₂O): δ: 1.3-2.0 (m, 32H), 2.6-3.25 (m, 28H), 3.6-3.95 (m, 8H), 7.0-7.4 (m, 20H)

### Example 42

### TETA(TyrOrn)₂(OrnOrn)₂ TFA Salt

The same procedures used in the steps 1 to 4 of Example 37 were repeated except that Z-L-Tyr(tBu)-OH was substituted for the Z-L-Phe-OH used in the step 1 to thus give an intended compound.

MS (ESI): m/z: 1157 [M+H]⁺

¹H-NMR (D₂O): δ: 6.97-7.31 (m, 4H), 6.69-6.78 (m, 4H), 4.28-4.53 (m, 4H), 3.54-3.91 (m, 4H), 2.60-3.54 (m, 28H), 1.51-1.93 (m, 24H)

### Example 43

### TETA(PheOrn)₂[Orn(Orn)Orn]₂ Hydrochloride

### Step 1: Deprotection and Condensation

After dissolving 1.36 g (0.97 mM) of the compound obtained in the step 2 of Example 37 in 20 mL of trifluoroacetic acid, the resulting solution was stirred for 2 hours. After the solvent was distilled off, the resulting residue was dissolved in a water-acetonitrile mixed solvent and then the solution was lyophilized. In a separate container, there were dissolved, in 10 mL of DMF, 650 mg of Boc-Orn(Boc)-OH, 810 mg of HATU and 290 mg of HOAt, the solution was then stirred at room temperature for 10 minutes, the compound previously obtained through the lyophilization was added to the solution and the mixture was stirred through the night. After the completion of the reaction, the same operations used in the step 1 of Example 1 were repeated to thus give an intermediate.

Yield: 0.80 g

### Step 2: Deprotection and Condensation

The compound obtained in the foregoing step 1 was dissolved in 30 mL of methanol, and the reaction was continued within a hydrogen atmosphere in the presence of a catalytic amount of Pd/C through the night. After the catalyst was filtered off, the solvent was distilled off to thus give an intermediate. In a separate container, there were dissolved, in 10 mL of DMF, 0.70 g (2.10 mM) of Boc-Orn(Boc)-OH, 0.88 g (2.31 mM) of HATU and 0.31 g (2.31 mM) of HOAt, 0.37 mL of TEA was then added to the solution and the mixture was then stirred at room temperature for 10 minutes. Thereafter, the intermediate previously prepared was added to the mixture and the mixture was stirred through the night. After the completion of the reaction, the same operations used in the step 1 of Example 1 were repeated to thus give an intermediate.

### Step 3: Deprotection

The compound obtained in the foregoing step 2 was subjected to Deprotection with 20 mL of trifluoroacetic acid and then the solvent was distilled off. The resulting residue was purified by the reversed phase high-performance liquid chromatography and the fraction containing an intended product was lyophilized to thus give an intermediate.

Yield: 0.48 g

### Step 4: Ion-Exchanging and Conversion into Hydrochloride

The TFA salt obtained in the foregoing step 3 was subjected to the same operations used in the step 3 of Example 1 to thus give an intended compound.

Yield: 0.45 g

MS (ESI): m/z: 677 [M+2H]²⁺

¹H-NMR (D₂O): δ: 7.14-7.38 (m, 10H), 4.46-4.64 (m, 4H), 3.83-4.11 (m, 6H), 2.55-3.67 (m, 32H), 1.49-1.95 (m, 32H)

### Example 44

### TETA(PheOrn)₂(δOrnOrn)₂ Hydrochloride

### Step 1: Condensation

There were dissolved, in DMF, 1.59 g of Boc-Orn(Z)-OH, 1.81 g of HATU and 0.65 g of HOAt and then 2.4 mL of TEA was added to the resulting mixture. After stirring the mixture for 10 minutes, 2.06 g of the compound obtained in the step 1 of Example 37 was added thereto and the mixture was stirred through the night. After the completion of the reaction, the same operations used in the step 1 of Example 1 were repeated to thus give an intermediate.

Yield: 2.0 g

### Step 2: Deprotection and Condensation

The compound obtained in the foregoing step 1 was dissolved in 30 mL of methanol, and the reaction was continued within a hydrogen atmosphere in the presence of a catalytic amount of Pd/C through the night. After the catalyst was once filtered off, the reaction components were again charged, the reaction system was stirred through the night, the catalyst was filtered off and the solvent was distilled off. There were dissolved, in 15 mL of DMF, 1.99 g (6.0 mM) of Boc-Orn(Boc)-OH, 2.51 g (6.0 mM) of HATU and 0.90 g (6.0 mM) of HOAt and then 1.05 mL of TEA was added to the solution. After stirring for 10 minutes, the previously prepared compound was added to this reaction system and the mixture was stirred through the night. After the completion of the reaction, the same operations used in the step 1 of Example 1 were repeated to thus give an intermediate.

### Step 3: Deprotection

The compound obtained in the foregoing step 2 was dissolved in 20 mL of trifluoroacetic acid and the solution was stirred at room temperature for one hour. The solvent was distilled off, the resulting residue was purified by the reversed phase high-performance liquid chromatography and the fraction containing an intended product was lyophilized to thus give an intermediate.

### Step 4: Ion-Exchanging and Conversion into Hydrochloride

After desalting the product obtained in the foregoing step using an ion-exchange resin like the step 3 of Example 1, hydrochloric acid was added in an amount corresponding to 18% by mass and then the mixture was lyophilized to thus give an intended compound.

Yield: 1.50 g

MS (ESI): m/z: 1112 [M+H]⁺

¹H-NMR (D₂O): δ: 7.16-7.40 (m, 10H), 4.23-4.57 (m, 4H), 3.85-4.01 (m, 4H), 2.82-3.58 (m, 28H), 1.48-1.98 (m, 24H)

### Example 45

### TETA(Phe-δOrnOrn)₅ Hydrochloride

### Step 1: Condensation

There was dissolved, in 15 mL of dichloromethane, 474 μL (2.5 mM) of tetraethylene pentamine, then 1.98 g (5 mM) of Z-Phe-OSu was added to the solution and the mixture was stirred at room temperature overnight. After the completion of the reaction, the solvent was distilled off, the resulting residue was purified by the reversed phase high-performance liquid chromatography and the fraction containing an intended product was lyophilized to thus give an intermediate.

Amount of the intermediate obtained: 909 mg (0.82 mM) (Yield: 33%)

MS (ESI): m/z: 751 [M+H]⁺

### Step 2: Condensation

There were dissolved, in 10 mL of DMF, 821 mg (2.74 mM) of Z-Phe-OH, 1.04 g (2.74 mM) of HATU and 373 mg (2.74 mM) of HOAt. To this solution, there were added 909 mg (0.82 mM) of the compound obtained in the foregoing step 1 and 9554 (6.85 mM) of TEA and the resulting mixture was stirred at room temperature through the night. After the completion of the reaction, the same operations used in the step 1 of Example 1 were repeated to thus give an intermediate.

Amount of the intermediate obtained: 837 mg (0.51 mM) (Yield: 63%)

MS (ESI): m/z: 798 [M+2H]²⁺

### Step 3: Deprotection

There was added 84 mg of 10% Pd/C to 837 mg (0.51 mM) of the compound obtained in the foregoing step 2 and the resulting mixture was dissolved and dispersed in 6 mL of methanol. After replacing the atmosphere of the reaction system with hydrogen gas, the system was stirred at room temperature overnight. After the Pd/C was filtered off, the solvent was distilled off, the resulting residue was purified by the reversed phase high-performance liquid chromatography and the fraction containing an intended product was lyophilized to thus give an intermediate.

Amount of the intermediate obtained: 528 mg (0.35 mM) (Yield: 68%)

MS (ESI): m/z: 925 [M+H]⁺

### Step 4: Condensation

There were dissolved, in 10 mL of DMF, 712 mg (1.94 mM) of Boc-Orn(Z)-OH, 737 mg (1.94 mM) of HATU and 265 mg (1.94 mM) of HOAt. To this solution, there were added 528 mg (0.35 mM) of the compound obtained in the foregoing step 3 and 677 μL (4.86 mM) of TEA and the resulting mixture was stirred at room temperature through the night. After the completion of the reaction, the same operations used in the step 1 of Example 1 were repeated to thus give an intended compound.

Amount of the compound obtained: 778 mg (0.29 mM) (Yield: 83%)

MS (ESI): m/z: 889 [M+3H]³⁺

### Step 5: Deprotection

There was added 77 mg of 10% Pd/C to 778 mg (0.29 mM) of the compound obtained in the foregoing step 4 and the resulting mixture was dissolved and dispersed in 6 mL of methanol. After replacing the atmosphere of the reaction system with hydrogen gas, the system was stirred at room temperature overnight. After the Pd/C was filtered off, the solvent was distilled off to thus give an intermediate.

Amount of the intermediate obtained: 578 mg (0.29 mM)

MS (ESI): m/z: 998 [M+2H]²⁺

### Step 6: Condensation

There were dissolved, in 10 mL of DMF, 712 mg (1.94 mM) of Boc-Orn(Z)OH, 737 mg (1.94 mM) of HATU and 265 mg (1.94 mM) of HOAt. To this solution, there were added 578 mg (0.29 mM) of the compound obtained in the foregoing step 3 and 677 μL (4.86 mM) of TEA and the resulting mixture was stirred at room temperature through the night. After the completion of the reaction, the same operations used in the step 1 of Example 1 were repeated to thus give an intermediate.

Amount of the intermediate obtained: 578 mg (0.15 mM) (Yield: 53%)

MS (ESI): m/z: 935 [M+4H]⁴⁺

### Step 7: Deprotection

There was added 57 mg of 10% Pd/C to 578 mg (0.15 mM) of the compound obtained in the foregoing step 6 and the resulting mixture was dissolved and dispersed in 5 mL of methanol. After replacing the atmosphere of the reaction system with hydrogen gas, the system was stirred at room temperature overnight. After the Pd/C was filtered off, the solvent was distilled off to thus give an intermediate.

MS (ESI): m/z: 767 [M+4H]⁴⁺

### Step 8: Deprotection

There was dissolved, in 5 mL of trifluoroacetic acid, 459 mg (0.15 mM) of the compound obtained in the foregoing step 7 and the resulting solution was stirred for one hour. The solvent was distilled off, the resulting residue was purified by the reversed phase high-performance liquid chromatography and the fraction containing an intended product was lyophilized to thus give an intermediate.

Amount of the intermediate obtained: 311 mg (0.08 mM) (Yield: 55% calculated for the two steps)

MS (ESI): m/z: 689 [M+3H]³⁺

### Step 9: Ion-Exchanging and Conversion into Hydrochloride

After desalting 311 mg (0.08 mM) of the compound obtained in the foregoing step 8 using an ion-exchange resin like the step 3 of Example 1, hydrochloric acid was added in an amount corresponding to 18% by mass and then the mixture was lyophilized to thus give an intended compound.

Amount of the compound obtained: 187 mg (0.07 mM)

### Example 46

### TETA(D-PheOrn)₂(OrnOrn)₂ Hydrochloride

### Step 1: Condensation

There were dissolved, in 10 mL of dichloromethane, 2.39 g (8.0 mM) of Z-D-Phe-OH and 1.30 g (8.0 mM) of CDI and the solution was stirred at room temperature for one hour. Then 568 mg (4.0 mM) of triethylene tetramine was added to the solution and the mixture was stirred overnight. After the completion of the reaction, the same procedure for purification used in the step 1 of Example 1 was repeated to thus give an intermediate.

Amount of the intermediate obtained: 2.45 mg (2.61 mM) (Yield: 65%)

### Step 2: Condensation

A solution was prepared by dissolving, in 5 mL of DMF, 2.20 g (6.0 mM) of Z-Orn(Boc)-OH, 2.28 g (6.0 mM) of HATU and 816 mg (6.0 mM) of HOAt. To this solution, there were added 2.45 g (2.61 mM) of the compound obtained in the foregoing step 1 and 2.07 g (12 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 2.0 mg (1.42 mM) (Yield: 54%)

### Step 3: Deprotection

To 2.0 g (1.42 mM) of the compound obtained in the foregoing step 2, there were added 1.0 g of 10% Pd/C and 40 mL of methanol, and the resulting mixture was stirred at room temperature overnight in a hydrogen gas atmosphere. The reaction solution was filtered through cerite and then the solvent was distilled off to thus give an intermediate.

Yield: Quantitative

### Step 4: Condensation

A solution was prepared by dissolving, in 5 mL of DMF, 2.10 g (6.3 mM) of Boc-Orn(Boc)-OH, 2.40 g (6.3 mM) of HATU and 852 mg (6.3 mM) of HOAt. To this solution, there were added the compound obtained in the foregoing step 3 and 2.17 g (12 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 2.4 g (1.39 mM) (Yield: 98%)

### Step 5: Deprotection

To the compound obtained in the foregoing step 4, there was added 5 mL of trifluoroacetic acid and the resulting mixture was stirred at room temperature for one hour. The solvent was distilled off under a reduced pressure, water was then added to the resulting residue and the aqueous mixture was lyophilized to thus remove the trifluoroacetic acid.

### Step 6: Desalting and Conversion into Hydrochloride

The compound obtained in the foregoing step 5 was subjected to the same treatments used in the step 3 of Example 1 to thus give an intended compound.

Amount of the compound obtained: 827 mg

MS (ESI): m/z: 563 [M+2H]²⁺

### Example 47

### TETA(TrpOrn)₂(OrnOrn)₂ Hydrochloride

### Step 1: Condensation

Z-Trp-OSu (3.85 g, 8.0 mM) was dissolved in 10 mL of dichloromethane, then 568 mg (4.0 mM) of triethylene tetramine was added to the solution and the mixture was stirred overnight. After the completion of the reaction, ether was added to the reaction system, the precipitates formed were recovered through filtration and washed twice with ether. The precipitates were dried under a reduced pressure to thus give an intermediate.

Yield: Quantitative

### Step 2: Condensation

A solution was prepared by dissolving, in 7 mL of DMF, 2.93 g (8.0 mM) of Z-Orn(Boc)-OH, 3.04 g (8.0 mM) of HATU and 1.09 g (8.0 mM) of HOAt, then there were added, to the solution, the compound obtained in the foregoing step 1 and 2.76 mL (16 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 2.04 g (1.30 mM) (Yield: 33%)

### Step 3: Deprotection

To 2.04 g (1.30 mM) of the compound obtained in the foregoing step 2, there were added 1.0 g of 10% Pd/C and 40 mL of methanol, and the resulting mixture was stirred at room temperature overnight in a hydrogen gas atmosphere. The reaction solution was filtered through cerite and then the solvent was distilled off to thus give an intermediate.

Yield: Quantitative

### Step 4: Condensation

A solution was prepared by dissolving, in 7 mL of DMF, 1.90 g (5.8 mM) of Boc-Orn(Boc)-OH, 2.2 g (5.8 mM) of HATU and 778 mg (5.8 mM) of HOAt, then there were added, to the solution, the compound obtained in the foregoing step 3 and 2.0 mL (11.6 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 2.0 g (0.97 mM) (Yield: 74%)

MS (ESI): m/z: 1103 [M+2H]²⁺

### Step 5: Deprotection

Trifluoroacetic acid (5 mL) was added to the compound obtained in the foregoing step 4 and the resulting mixture was stirred at room temperature for one hour. The solvent was distilled off under a reduced pressure, water was then added to the resulting residue and the aqueous mixture was lyophilized to thus remove the trifluoroacetic acid.

### Step 6: Ion-Exchanging and Conversion into Hydrochloride

The compound obtained in the foregoing step 5 was subjected to the same operations used in the step 3 of Example 1 to thus give an intended compound.

Yield: 379 mg

MS (ESI): m/z: 602 [M+2H]²⁺

### Example 48

### TAA(PheOrnOrn)₃ Hydrochloride

### Step 1: Condensation and Deprotection

There were dissolved, in methylene chloride (30 mL), 1.49 g (9.0 mM) of CDI and 2.69 g (9.0 mM) of Z-L-Phe and the solution was stirred at room temperature for 30 minutes. To the solution, there was added 0.450 mL (3.0 mM) of tris(2-aminoethyl)-amine and the mixture was stirred at room temperature through the night. A water-acetone mixed solvent was added to the solution to form a suspension and the solids thus separated out from the mixture were recovered through filtration.

The solid thus obtained were dissolved in tetrahydrofuran (30 mL) and methanol (30 mL), followed by the addition of a catalytic amount of Pd/C and the stirring of the resulting mixture in a hydrogen gas atmosphere through the night. After the completion of the reaction, the catalyst was filtered off and then the solvent was distilled off to thus give an intermediate. There were separately dissolved, in 15 mL of DMF, 1.35 g (3.67 mM) of Z-Orn(Boc)-OH, 0.55 g of HOAt and 1.53 g of HATU, then 0.85 mL of TEA was added to the mixture and the resulting mixture was stirred for 10 minutes. The intermediate previously prepared was dissolved in 10 mL of DMF, the resulting solution was added to the separately prepared solution and the resulting mixture was stirred through the night. Ethyl acetate and an aqueous 1N-sodium hydroxide solution were added to the mixture and the mixture was extracted twice with ethyl acetate. After washing the extract with, in order, an aqueous 1N-sodium hydroxide solution and an aqueous saturated common salt solution, the resulting organic phase was dried over anhydrous sodium sulfate. The solvent was distilled off, the resulting solids were suspended in acetone and the solids were recovered through filtration to thus give 2.76 g of an intermediate. The solids (2.76 g) thus obtained were dissolved in a acetic acid (30 mL):methanol (20 mL) mixed solvent, a catalytic amount of Pd/C was added to the solution and the resulting mixture was stirred in a hydrogen gas atmosphere through the night. After the catalyst was removed through filtration, the resulting residue was dissolved in a methanol (20 mL):water (20 mL) mixed solvent, a catalytic amount of Pd/C was added to the solution and the resulting mixture was stirred in a hydrogen gas atmosphere through the night. Then the catalyst was filtered off and the solvent was distilled off. The resulting residue was dissolved in water, subjected to desalting with an anionic ion-exchange resin and then the resulting aqueous solution was lyophilized to thus give an intermediate (1.82 g).

There were dissolved, in 10 mL of DMF, 1.69 g (5.07 mM) of Boc-Orn(Boc)-OH, 0.76 g of HOAt and 2.12 g of HATU, then 0.94 mL of TEA was added to the solution and the resulting mixture was stirred for 10 minutes.

To the resulting mixture, there was added a solution of the intermediate (1.82 g) previously formed in 5 mL of DMF and the mixture was stirred through the night. The solvent was distilled off, ethyl acetate and an aqueous 1N-sodium hydroxide solution were added to the resulting residue and the mixture was extracted twice with ethyl acetate. After washing the extract with, in order, an aqueous 1N-sodium hydroxide solution and an aqueous saturated common salt solution, the resulting organic phase was dried over anhydrous sodium sulfate. The solvent was distilled off and the resulting residue was purified by the same procedures used in the step 1 of Example 1 to thus give an intermediate.

### Step 2: Deprotection

The compound obtained in the foregoing step 1 was dissolved in 20 mL of trifluoroacetic acid and the solution was stirred for 2 hours. The same purification procedures used in the step 1 of Example 1 were repeated to thus give a compound.

### Step 3: Ion-Exchanging and Conversion into Hydrochloride

The compound prepared above was subjected to desalting with an ion-exchange resin like the step 3 of Example 1 to thus obtain the title compound.

Yield: 0.194 g

¹H-NMR (D₂O): δ: 7.35-7.13 (m, 15H), 4.47-4.27 (m, 6H), 4.06-3.89 (m, 3H), 3.33-2.84 (m, 30H), 2.50-1.53 (24H)

### Example 49

### TETA(Phe-αOrnOrn)₄ Hydrochloride

### Step 1: Condensation

There were dissolved, in 12 mL of DMF, 1.13 g (3.1 mM) of Z-Orn(Boc)OH, 1.17 g (3.1 mM) of HATU and 422 mg (3.1 mM) of HOAt. To this solution, there were added 1.44 g (0.70 mM) of the compound obtained in the step 4 of Example 40: TETA[Phe-Orn(Boc)]₄.4TFA, 1080 μL (7.75 mM) of TEA and the mixture was stirred at room temperature through the night. After the completion of the reaction, the same operations used in the step 1 of Example 1 were repeated to thus give an intermediate.

Amount of the intermediate obtained: 1.64 g (0.55 mM) (Yield: 79%)

MS (ESI): m/z: 995 [M+3H]³⁺

### Step 2: Deprotection

To 1.64 g (0.55 mM) of the compound obtained in the foregoing step 1, there was added 164 mg of 10% Pd/C and the resulting mixture was dispersed and dissolved in 15 mL of methanol. After replacing the atmosphere of the reaction system with hydrogen, the reaction system was stirred at room temperature overnight. The Pd/C was filtered off, the solvent was distilled off, the resulting residue was purified by the reversed phase high-performance liquid chromatography and the fraction containing an intended product was lyophilized to thus give the intended compound.

Amount of the product obtained: 869 mg (0.29 mM) (Yield: 54%)

MS (ESI): m/z: 816 [M+3H]³⁺

### Step 3: Deprotection

The compound (869 mg, 0.29 mM) obtained in the foregoing step 2 was dissolved in 8 mL of trifluoroacetic acid and the solution was stirred for one hour. The solvent was distilled off, the resulting residue was purified by the reversed phase high-performance liquid chromatography and the fraction containing an intended product was lyophilized to thus give an intermediate.

Amount of the intermediate obtained: 773 mg

MS (ESI): m/z: 550 [M+3H]³⁺

### Step 4: Ion-Exchanging and Conversion into Hydrochloride

The compound (773 mg) obtained in the foregoing step 3 was subjected to the same operations used in the step 3 of Example 1 to thus give an intended compound.

Amount of the compound obtained: 470 mg (0.24 mM)

### Example 50

### TETA(OrnOrn)₂(PheOrn)₂ Hydrochloride

### Step 1: Condensation

There was dissolved, in 50 mL of dichloromethane, 4.2 g (7.81 mM) of Boc-(Orn(TFA))₂-OH and 1.265 g (7.81 mM) of CDI and the solution was stirred at room temperature for one hour. Then 0.583 mL (3.91 mM) of triethylene tetramine was added to the solution and the mixture was stirred overnight. After the completion of the reaction, the solvent was distilled off under a reduced pressure, the resulting residue was purified by the reversed phase high-performance liquid chromatography and the fraction containing an intended product was lyophilized to thus give an intermediate.

Amount of the intermediate obtained: 1.0 g (0.55 mM) (Yield: 14%)

### Step 2: Condensation

To the mixture comprising 412 mg (1.375 mM) of Z-Phe-OH, 522 mg (1.375 mM) of HATU, 190 mg (1.375 mM) of HOAt and 10 mL of DMF, there was added a solution of 1.0 g (0.55 mM) of the compound obtained in the foregoing step 1 and 0.46 mL (3.3 mM) of TEA in 10 mL of DMF and the resulting mixture was stirred at room temperature overnight. After the completion of the reaction, the same post-treatments and purification step used in the step 1 of Example 1 were repeated to thus give an intermediate.

Amount of the intermediate obtained: 800 mg (0.46 mM) (Yield: 84%)

### Step 3: Deprotection, Condensation and Deprotection

To 800 mg (0.46 mM) of the compound obtained in the foregoing step 2, there were added 1 g of 10% Pd/C and 20 mL of methanol, and the resulting mixture was stirred at room temperature overnight in a hydrogen gas atmosphere. The reaction solution was filtered through cerite and then the solvent was distilled off to thus give an intermediate. A solution was separately prepared by dissolving, in 5 mL of DMF, 360 mg (1.1 mM) of Boc-Orn(Boc)-OH, 420 mg (1.1 mM) of HATU and 150 mg (1.1 mM) of HOAt, then the compound obtained above and 0.64 mL (2.3 mM) of TEA were added to the solution and the resulting mixture was stirred at room temperature overnight. After the completion of the reaction, the reaction system was subjected to the same post-treatment used in the step 1 of Example 1, there were added, to the reaction system, 780 mg (7.36 mM) of sodium carbonate, 20 mL of methanol and 4 mL of water and the mixture was stirred overnight. The solvent was distilled off, the resulting residue was purified by the reversed phase high-performance liquid chromatography and the fraction containing an intended product was lyophilized to thus give an intermediate.

### Step 4: Deprotection

Trifluoroacetic acid (5 mL) was added to the compound obtained in the foregoing step 3 and the mixture was stirred at room temperature for one hour. The solvent was distilled off under a reduced pressure, water was added to the resulting residue and then the aqueous mixture was lyophilized to thus remove the trifluoroacetic acid.

### Step 5: Ion-Exchanging and Conversion into Hydrochloride

The compound obtained in the foregoing step 4 was subjected to the same operations used in the step 3 of Example 1 to thus give an intended compound.

Yield: 250 mg

MS (ESI): m/z: 376 [M+3H]³⁺

### Example 51

### TETA[Phe-Orn(Orn)₂]₄ Hydrochloride

### Step 1: Deprotection

There were dissolved, in 8 mL of trifluoroacetic acid, 1.44 g (0.7 mM) of the compound: TETA[Orn(Boc)]₄.4TFA prepared in the step 4 of Example 40 and the solution was stirred for one hour. The solvent was distilled off, water was added to the resulting residue to give a solution and the solution was lyophilized to give an intended compound.

### Step 2: Condensation

There were dissolved, in 15 mL of DMF, 2.25 g (6.16 mM) of Z-Orn(Boc)OH, 2.33 g (6.16 mM) of HATU and 838 mg (6.16 mM) of HOAt. To this solution, there were added 1.47 g (0.7 mM) of the compound obtained in the foregoing step 1 and 2.14 mL (15.4 mM) of TEA and the resulting mixture was stirred through the night. After the completion of the reaction, the same post-treatment used in the step 1 of Example 1 was repeated and the product was used in the subsequent step without isolating and purifying the same.

### Step 3: Deprotection

To the mixture prepared in the foregoing step 3, there was added 10% Pd/C and the resulting mixture was then dispersed and dissolved in 15 mL of methanol. After replacing the atmosphere of the reaction system with hydrogen, the system was stirred at room temperature overnight. The Pd/C was filtered off, then the solvent was distilled off, the resulting residue was purified by the reversed phase high-performance liquid chromatography and the fraction containing an intended product was lyophilized to thus give an intermediate.

Amount of the product obtained: 1.35 g (0.28 mM) (Yield: 41% calculated for the three steps)

MS (ESI): m/z: 969 [M+3H]³⁺

### Step 4: Deprotection

The compound (1.35 g, 0.28 mM) obtained in the foregoing step 3 was dissolved in 8 mL of trifluoroacetic acid and the solution was stirred for one hour. The solvent was distilled off and the resulting residue was purified by the reversed phase high-performance liquid chromatography, followed by the lyophilization to thus give the intended compound.

Yield: 1.26 g (0.32 mM)

MS (ESI): m/z: 526 [M+4H]⁴⁺

### Step 5: Ion-Exchanging and Conversion into Hydrochloride

The compound (311 mg, 0.08 mM) obtained in the foregoing step 4 was subjected to the same operations used in the step 3 of Example 1 to thus give an intended compound.

Yield: 768 mg (0.30 mM)

¹H-NMR (D₂O): δ: 1.375-2.0 (m, 48H), 2.4-3.5 (m, 44H), 3.6-4.6 (m, 16H), 7.0-7.35 (m, 20H)

### Example 52

### TETA(TyrOrn)₂(OrnOrn)₂ Hydrochloride

### Step 1: Ion-Exchanging and Conversion into Hydrochloride

The compound obtained in Example 34 was subjected to the same operations used in the step 3 of Example 1 to thus give an intended compound.

Yield: 57 mg

¹H-NMR (D₂O): δ: 7.15-7.30 (m, 4H), 6.82-6.96 (m, 4H), 4.50-4.75 (m, 4H), 4.03-4.21 (m, 4H), 2.75-3.92 (m, 28H), 1.61-2.11 (m, 24H)

### Example 53

### TETA[Dab(Dab)₂]₄ Hydrochloride

### Step 1: Condensation

There were suspended, in 50 mL of dichloromethane, 2.04 g (5.8 mM) of Z-Dab(Boc)-OH and 940 mg (5.8 mM) of CDI and the resulting suspension was stirred at room temperature for one hour. To the suspension, there was added 0.432 mL (2.9 mM) of triethylene tetramine and the mixture was stirred overnight. After the completion of the reaction, the solvent was distilled off under a reduced pressure. To a mixture comprising 2.25 g (6.38 mM) of Z-Dab(Boc)-OH, 2.42 g (6.38 mM) of HATU, 8.68 mg (6.38 mM) of HOAt and 10 mL of DMF, there was added a solution obtained by dissolving, in 10 mL of DMF, the residue obtained above and 1.21 mL (8.7 mM) of TEA. After the completion of the reaction, the same post-treatments and purification step used in the step 1 of Example 1 were repeated to thus give an intermediate.

Amount of the intermediate obtained: 1.39 g (0.94 mM) (Yield: 32%)

### Step 2: Deprotection and Condensation

To 1.39 g (0.94 mM) of the compound obtained in the foregoing step 1, there was added 10% Pd/C and the resulting mixture was dissolved and dispersed in 50 mL of methanol. After replacing the atmosphere of the reaction system with hydrogen, the system was stirred at room temperature overnight. The Pd/C was filtered off and then the solvent was distilled off. To a mixture comprising 2.65 g (7.52 mM) of Z-Dab(Boc)-OH, 2.86 g (7.52 mM) of HATU, 1.0 g (7.52 mM) of HOAt and 10 mL of DMF, there was added a solution obtained by dissolving the residue obtained above in 10 mL of DMF and 2.1 mL (15 mM) of TEA. After the completion of the reaction, the same post-treatments used in the step 1 of Example 1 were repeated to thus give an intermediate.

### Step 3: Deprotection

To 1.39 g (0.94 mM) of the compound obtained in the foregoing step 1, there was added 10% Pd/C and the resulting mixture was dissolved and dispersed in 50 mL of methanol. After replacing the atmosphere of the reaction system with hydrogen, the system was stirred at room temperature overnight. The Pd/C was filtered off and then the solvent was distilled off. Trifluoroacetic acid (5 mL) was added to the resulting residue and the mixture was stirred at room temperature for one hour. The solvent was distilled off under a reduced pressure, followed by the addition of water to the resulting residue and the lyophilization of the aqueous mixture to thus remove the trifluoroacetic acid.

### Step 4: Ion-Exchanging and Conversion into Hydrochloride

The compound obtained in the foregoing step 3 was subjected to the same operations used in the step 3 of Example 1 to thus give an intended compound.

Yield: 172 mg (0.10 mM)

¹H-NMR (D₂O): δ: 1.6-2.2 (m, 24H), 2.8-3.9 (m, 48H), 4.15-4.3 (m, 4H)

### Example 54

### TETA[Lys(Dab)₂]₄ TFA Salt

### Step 1: Condensation

There was added 6 mL of DMF to a mixture comprising 15.6 g (29.49 mM) of Boc-Lys(Boc)OH DCHA, 11.2 g (29.49 mM) of HBTU and 4.5 g (29.49 mM) of HOBt/H₂O followed by the stirring of the mixture to thus dissolve the components. To this solution, there were added 1.0 mL (6.7 mM) of triethylene tetramine and 4.1 mL (29.49 mM) of TEA and the resulting mixture was stirred at room temperature overnight. After the completion of the reaction, the same post-treatments and purification step used in the step 1 of Example 1 were repeated to thus give an intermediate.

Amount of the intermediate obtained: 6.01 g (4.12 mM) (Yield: 62%)

### Step 2: Deprotection

The compound obtained in the foregoing step 1 was dissolved in 25 mL of trifluoroacetic acid and the solution was stirred for one hour. The solvent was distilled off to thus give an intermediate.

### Step 3: Condensation

To a mixture comprising 2.16 g (6.15 mM) of Z-Dab(Boc)-OH, 2.34 g (6.15 mM) of HATU, 836 mg (6.15 mM) of HOAt and 10 mL of DMF, there was added a solution obtained by dissolving 1.15 g (0.732 mM) of the compound obtained in the foregoing step 2 in 1.6 mL (11.5 mM) of TEA and 10 mL of DMF. After the completion of the reaction, the same post-treatments used in the step 1 of Example 1 were repeated, 10% Pd/C was added to the resulting residue and the mixture was dissolved and dispersed in 50 mL of methanol. After replacing the atmosphere of the reaction system with hydrogen, the system was stirred at room temperature overnight. After the Pd/C was filtered off, the solvent was distilled off. The same post-treatments and purification step used in the step 1 of Example 1 were repeated to thus give an intermediate.

### Step 4: Deprotection

The compound (520 mg, 0.16 mM) obtained in the foregoing step 3 was dissolved in 5 mL of trifluoroacetic acid and the solution was stirred for one hour. The solvent was distilled off, the resulting residue was purified by the reversed phase high-performance liquid chromatography and the fraction containing an intended product was lyophilized to thus give the intended compound.

Amount of the compound obtained: 105 mg (0.032 mM) (Yield: 20%)

MS (ESI): m/z: 487 [M+3H]³⁺

### Example 55

### TETA(D-Phe-Orn-Orn)₄ Hydrochloride

### Step 1: Condensation

There were dissolved, in 10 mL of DMF, 5.26 g (17.6 mM) of Z-D-Phe-OH, 6.69 g (17.6 mM) of HATU and 2.39 g (17.6 mM) of HOAt and the solution was stirred at room temperature for one hour. Then 568 mg (4.0 mM) of TETA was added to the solution and the mixture was stirred overnight. After the completion of the reaction, the same operations used in the step 1 of Example 1 were repeated to thus give an intermediate.

Amount of the intermediate obtained: 1.10 g (0.87 mM) (Yield: 22%)

### Step 2: Deprotection

To 1.10 g (0.87 mM) of the compound obtained in the foregoing step 1, there was added 0.5 g of 10% Pd/C and the resulting mixture was dissolved and dispersed in 20 mL of methanol and the solution was stirred at room temperature overnight in a hydrogen gas atmosphere. The reaction solution was filtered through cerite and then the solvent was distilled off to thus give an intermediate.

Yield: Quantitative

### Step 3: Condensation

To a solution obtained by dissolving, in 7 mL of DMF, 1.66 g (4.5 mM) of Z-Orn(Boc)-OH, 1.72 g (4.5 mM) of HATU and 6161 mg (4.5 mM) of HOAt, there were added the compound obtained in the foregoing step 2 and 781 μL (9.0 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 1.14 g (0.54 mM) (Yield: 62%)

### Step 4: Deprotection

To 1.10 g (0.87 mM) of the compound obtained in the foregoing step 3, there were added 0.5 g of 10% Pd/C and 20 mL of methanol and the resulting mixture was stirred at room temperature overnight in a hydrogen gas atmosphere. The reaction solution was filtered through cerite and the solvent was distilled off to thus give an intermediate.

Amount of the intermediate obtained: 809 mg (0.51 mM) (Yield: 94%)

### Step 5: Condensation

To a solution obtained by dissolving, in 4 mL of DMF, 743 mg (2.23 mM) of Boc-Orn(Boc)-OH, 850 mg (2.23 mM) of HATU and 304 mg (2.23 mM) of HOAt, there were added the compound obtained in the foregoing step 4 and 7694 (4.46 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 310 mg (0.11 mM) (Yield: 21%)

### Step 6: Deprotection

Trifluoroacetic acid (5 mL) was added to the compound obtained in the foregoing step 5 and the mixture was stirred at room temperature for one hour. The solvent was distilled off under a reduced pressure, followed by the addition of water to the resulting residue and the lyophilization of the aqueous mixture to thus remove the excess trifluoroacetic acid.

### Step 7: Ion-Exchanging and Conversion into Hydrochloride

The compound obtained in the foregoing step 6 was subjected to the same operations used in the step 3 of Example 1 to thus give an intended compound.

Yield: 100 mg

MS (ESI): m/z: 824 [M+2H]²⁺

### Example 56

### TETA (Trp-Orn-Dab)₄ Hydrochloride

### Step 1: Condensation

There were dissolved, in 10 mL of DMF, 5.95 g (17.6 mM) of Z-Trp-OH, 6.69 g (17.6 mM) of HATU and 2.39 g (17.6 mM) of HOAt and the solution was stirred at room temperature for one hour. Then 568 mg (4.0 mM) of triethylene tetramine was added to the solution and the mixture was stirred overnight. After the completion of the reaction, the same purification step used in the step 1 of Example 1 was repeated to thus give an intermediate.

Amount of the intermediate obtained: 3.97 g (2.78 mM) (Yield: 70%)

### Step 2: Deprotection

To 3.97 g (2.78 mM) of the compound prepared in the foregoing step 1, there were added 2.0 g of 10% Pd/C and 60 mL of methanol and the resulting mixture was stirred at room temperature overnight in a hydrogen gas atmosphere. The reaction liquid was filtered through cerite and then the solvent was distilled off to give an intermediate.

Amount of the intermediate obtained: 2.01 g (2.25 mM) (Yield: 81%)

### Step 3: Condensation

To a solution prepared by dissolving, in 10 mL of DMF, 3.62 g (9.9 mM) of Z-Orn(Boc)-OH, 3.76 g (9.9 mM) of HATU and 1.35 g (9.9 mM) of HOAt, there were added the compound obtained in the foregoing step 2 and 3.41 mL (19.8 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 3.70 g (1.62 mM) (Yield: 72%)

### Step 4: Deprotection

To 3.70 g (1.62 mM) of the compound obtained in the foregoing step 3, there were added 1.5 g of 10% Pd/C and 60 mL of methanol and the resulting mixture was stirred at room temperature overnight in a hydrogen gas atmosphere. The reaction liquid was filtered through cerite and then the solvent was distilled off to give an intermediate.

Amount of the intermediate obtained: 2.80 g (1.60 mM) (Yield: 99%)

### Step 5: Condensation

To a solution prepared by dissolving, in 7 mL of DMF, 665 mg (2.16 mM) of Boc-Dab(Boc)-OH, 821 mg (2.16 mM) of HATU and 294 mg (2.16 mM) of HOAt, there were added 782 mg (0.45 mM) of the compound obtained in the foregoing step 4 and 755 μL (4.32 mM) of DIEA and the mixture was stirred at room temperature overnight. After the completion of the reaction, the same post-treatments and purification step used in the step 1 of Example 1 were repeated to thus give an intermediate.

Amount of the intermediate obtained: 437 mg (0.15 mM) (Yield: 34%)

### Step 6: Deprotection

Trifluoroacetic acid (5 mL) was added to the compound obtained in the foregoing step 5 and the mixture was stirred at room temperature for one hour. The solvent was distilled off under a reduced pressure, followed by the addition of water to the resulting residue and the lyophilization of the aqueous mixture to thus remove the trifluoroacetic acid.

### Step 7: Ion-Exchanging and Conversion into Hydrochloride

The compound obtained in the foregoing step 6 was subjected to the same operations used in the step 3 of Example 1 to thus give an intended compound.

Yield: 205 mg

MS (ESI): m/z: 874 [M+2H]²⁺

### Example 57

### TETA(PheOrn)₄ Hydrochloride

### Step 1: Condensation

There was added 8 mL of DMF to a mixture comprising 1.11 g (3.34 mM) of BocOrn(Boc)OH, 1.39 g (3.34 mM) of HATU and 489 mg (3.34 mM) of HOAt and the resulting mixture was stirred to dissolve the mixture in the DMF. To this solution, there were added 600 mg (0.817 mM) of the compound: TETA(Phe)₄ obtained in the step 2 of Example 40 and 570 μL (4.09 mM) of TEA and the mixture was stirred at room temperature overnight. After the completion of the reaction, the same post-treatments and purification step used in the step 1 of Example 1 was repeated to thus give an intermediate.

Amount of the intermediate obtained: 1.106 g (0.55 mM) (Yield: 67.9%)

MS (ESI): m/z: 997 [M+2H]²⁺

### Step 2: Deprotection

Trifluoroacetic acid (6 mL) was added to 1.106 g (0.55 mM) of the compound prepared in the foregoing step 1 and the resulting mixture was stirred at room temperature for one hour. The solvent was distilled off under a reduced pressure and the resulting residue was purified by the reversed phase high-performance liquid chromatography and the fraction containing an intended product was lyophilized to thus give an intermediate.

MS (ESI): m/z: 596 [M+2H]²⁺

### Step 3: Ion-Exchanging and Conversion into Hydrochloride

The compound obtained in the foregoing step 2 was subjected to the same operations used in the step 3 of Example 1 to thus give an intended compound.

Amount of the compound obtained: 531.8 mg (0.365 mM) (Yield: 65.9%)

MS (ESI): m/z: 596 [M+2H]²⁺

### Example 58

### TETA(OrnOrn)₂(ChaOrn)₂ Hydrochloride

### Step 1: Condensation

There were dissolved, in 10 mL of dichloromethane, 3.22 g (8.8 mM) of Z-Orn(Boc)-OH and 1.43 g (8.8 mM) of CDI and the mixture was stirred at room temperature for one hour. Then 568 mg (4.0 mM) of TETA was added to the mixture followed by the stirring of the mixture overnight. After the completion of the reaction, the solvent was distilled off under a reduced pressure, there were added, to the resulting residue, a solution prepared by dissolving, in 30 mL of DMF, 4.3 g (8.8 mM) of Z-Cha-OH.DCHA, 3.3 g (8.8 mM) of HATU, 1.2 g (8.8 mM) of HOAt and 1.94 mL (17.6 mM) of DIEA and the resulting mixture was stirred at room temperature overnight. After the completion of the reaction, the same post-treatments and purification step used in the step 1 of Example 1 were repeated to thus give an intermediate.

Amount of the intermediate obtained: 2.27 g (1.60 mM) (Yield: 40%)

### Step 2: Deprotection

To 2.27 g (1.60 mM) of the compound obtained in the foregoing step 1, there were added 1.0 g of 10% Pd/C and 40 mL of methanol and the resulting mixture was stirred at room temperature overnight in a hydrogen gas atmosphere. The reaction liquid was filtered through cerite and then the solvent was distilled off to give an intermediate.

Yield: Quantitative

### Step 3: Condensation

To a solution prepared by dissolving, in 10 mL of DMF, a mixture comprising 1.32 g (4.0 mM) of Boc-Orn(Boc)-OH, 1.65 g (4.0 mM) of HCTU and 676 mg (4.0 mM) of Cl-HOBt, there were added 800 mg (0.91 mM) of the compound obtained in the foregoing step 2 and 1.38 mL (8.0 mM) of DIEA and the resulting mixture was stirred at room temperature overnight. After the completion of the reaction, the same post-treatments and purification step used in the step 1 of Example 1 was repeated to thus give an intermediate.

Amount of the intermediate obtained: 1.0 g (0.47 mM) (Yield: 51%)

### Step 4: Deprotection

Trifluoroacetic acid (5 mL) was added to the compound prepared in the foregoing step 3 and the resulting mixture was stirred at room temperature for one hour. The solvent was distilled off under a reduced pressure, followed by the addition of water to the resulting residue and the lyophilization of the aqueous mixture to thus remove the trifluoroacetic acid.

### Step 5: Ion-Exchanging and Conversion into Hydrochloride

The compound obtained in the foregoing step 4 was subjected to the same operations used in the step 3 of Example 1 to thus give an intended compound.

Yield: 484 mg

MS (ESI): m/z: 569 [M+2H]²⁺

### Example 59

### TETA(Orn)₂[Dab(Dab)₂]₂ Hydrochloride

### Step 1: Condensation

There were dissolved, in 8 mL of dichloromethane, 1.6 g (4.00 mM) of Z-Orn(Z)OH and 648 mg (4.00 mM) of CDI and the solution was stirred at room temperature for one hour. To this solution, there was added 2984 (2.00 mM) of triethylene tetramine and the mixture was stirred at room temperature through the night. The solvent was distilled off, the resulting residue was purified by the reversed phase high-performance liquid chromatography and the fraction containing an intended product was lyophilized to thus give an intermediate.

Amount of the intermediate obtained: 1.22 g (1.08 mM) (Yield: 54%)

MS (ESI): m/z: 911 [M+H]⁺

### Step 2: Condensation

There were dissolved, in 10 mL of DMF, 755 mg (2.37 mM) of Boc-Dab(Boc)OH, 910 mg (2.37 mM) of HATU and 323 mg (2.37 mM) of HOAt. To this solution, there were added 1.22 g (1.08 mM) of the compound obtained in the foregoing step 1 and 542 μL (3.88 mM) of TEA and the resulting mixture was stirred at room temperature through the night. After the completion of the reaction, the same operation used in the step 1 of Example 1 was repeated to thus give an intermediate.

Amount of the intermediate obtained: 814 mg (0.52 mM) (Yield: 49%)

MS (ESI): m/z: 756 [M+2H]²⁺

### Step 3: Deprotection

The compound (814 mg, 0.52 mM) prepared in the foregoing step 2 was dissolved in a mixed solvent containing 2 mL of trifluoroacetic acid and 5 mL of dichloromethane and the resulting solution was stirred for one hour. The solvent was distilled off, followed by the addition of water to the resulting residue to thus dissolve the compound and the lyophilization of the resulting solution to thus give an intended compound.

### Step 4: Condensation

There were dissolved, in 15 mL of DMF, 1.28 g (4.02 mM) of Boc-Dab(Boc)OH, 1.52 g (4.02 mM) of HATU and 548 mg (4.02 mM) of HOAt. To this solution, there were added the compound obtained in the foregoing step 3 and 542 μL (3.88 mM) of TEA and the mixture was stirred at room temperature through the night. After the completion of the reaction, the same operation used in the step 1 of Example 1 was repeated to thus give an intermediate.

Amount of the intermediate obtained: 1.22 g (0.52 mM) (Yield: 58%)

MS (ESI): m/z: 771 [M+3H]³⁺

### Step 5: Deprotection

There was added 122 mg of 10% Pd/C to 1.22 g (0.52 mM) of the compound obtained in the foregoing step 4 and the mixture was dissolved and dispersed in 6 mL of methanol. After replacing the atmosphere of the reaction system with hydrogen, the system was stirred at room temperature overnight. After the removal of the Pd/C through filtration, the solvent was distilled off, the resulting residue was purified by the reversed phase high-performance liquid chromatography and the fraction containing an intended product was lyophilized to thus give an intermediate.

Amount of the intermediate obtained: 640 mg (0.31 mM) (Yield: 61%)

MS (ESI): m/z: 592 [M+3H]³⁺

### Step 6: Deprotection

The compound (640 mg, 0.31 mM) obtained in the foregoing step 5 was dissolved in 5 mL of trifluoroacetic acid and the solution was stirred for one hour. The solvent was distilled off, the resulting residue was purified by the reversed phase high-performance liquid chromatography and the fraction containing an intended product was lyophilized to thus give an intermediate.

Amount of the intermediate obtained: 307 mg (0.13 mM) (Yield: 42%)

MS (ESI): m/z: 488 [M+2H]²⁺

### Step 7: Ion-Exchanging and Conversion into Hydrochloride

The compound (307 mg, 0.13 mM) obtained in the foregoing step 6 was subjected to the same operations used in the step 3 of Example 1 to thus give an intended compound.

Amount of the compound obtained: 155 mg (0.12 mM) (Yield: 95%)

¹H-NMR (D₂O): δ: 1.4-1.8 (m, 24H), 2.8-4.0 (m, 36H), 7.1-7.35 (m, 10H)

### Example 60

### TAA(TrpOrnOrn)₃ Hydrochloride

### Step 1: Condensation

There were dissolved, in 10 mL of DMF, 5.57 g (16.5 mM) of Z-Trp-OH, 6.25 g (16.5 mM) of HBTU and 2.52 g (16.5 mM) of HOBt and the solution was stirred at room temperature for one hour. Tris(2-aminoethyl)-amine (730 mg, 5 mM) was added to the solution and the mixture was stirred overnight. After the completion of the reaction, the same purification step used in the step 1 of Example 1 was repeated to thus give an intermediate.

Amount of the intermediate obtained: 4.86 g (4.39 mM) (Yield: 88%)

### Step 2: Deprotection

There were added 2.0 g of 10% Pd/C and 60 mL of methanol to 4.86 g (4.39 mM) of the compound obtained in the foregoing step 1 and the resulting mixture was stirred at room temperature overnight in a hydrogen gas atmosphere. The reaction liquid was filtered through cerite and then the solvent was distilled off to give an intermediate.

Yield: Quantitative

### Step 3: Condensation

The compound obtained in the foregoing step 2 and 5.24 mL (30.4 mM) of DIEA were added to a solution prepared by dissolving, in 10 mL of DMF, 5.55 g (15.2 mM) of Z-Orn(Boc)-OH, 6.27 g (15.2 mM) of HCTU and 2.57 g (15.2 mM) of Cl-HOBt and the mixture was stirred at room temperature overnight. After the completion of the reaction, water was added to the reaction system, followed by the filtration thereof and washing of the resulting solids with an ethyl acetate/ether mixed solvent. The solids were dried under a reduced pressure to give an intermediate.

Amount of the intermediate obtained: 6.70 g (3.83 mM) (Yield: 87%)

### Step 4: Deprotection

There were added 2.0 g of 10% Pd/C and 60 mL of methanol to 6.70 g (3.83 mM) of the compound obtained in the foregoing step 3 and the resulting mixture was stirred at room temperature overnight in a hydrogen gas atmosphere. The reaction liquid was filtered through cerite, the solvent was distilled off under a reduced pressure, the resulting residue was purified by the reversed phase high-performance liquid chromatography, followed by the lyophilization of the corresponding fraction to thus give an intended compound.

Amount of the compound obtained: 2.75 g (1.98 mM) (Yield: 52%)

### Step 5: Condensation

The compound (1.0 g, 0.72 mM) obtained in the foregoing step 4 and 820 μL (4.8 mM) of DIEA were added to a solution obtained by dissolving, in 5 mL of DMF, 789 mg (2.4 mM) of Z-Orn(Boc)-OH, 982 mg (2.4 mM) of HCTU and 402 mg (2.4 mM) of Cl-HOBt and the resulting mixture was stirred at room temperature overnight. After the completion of the reaction, the reaction liquid was diluted with ethyl acetate, washed with, in order, water, a 0.1M aqueous hydrochloric acid solution, an aqueous saturated sodium bicarbonate solution, and an aqueous saturated common salt solution, dried over sodium sulfate and then the solvent was distilled off. The resulting solids were washed with a mixed ethyl acetate/ether solvent. The solids were dried under a reduced pressure to give an intermediate.

Yield: Quantitative

### Step 6: Deprotection

Trifluoroacetic acid (5 mL) was added to the compound obtained in the foregoing step 5 and the mixture was stirred at room temperature for one hour. The solvent was distilled off under a reduced pressure, followed by the addition of water to the resulting residue and the lyophilization of the aqueous mixture to remove the trifluoroacetic acid and to thus obtain an intermediate.

### Step 7: Ion-Exchanging and Conversion into Hydrochloride

The compound obtained in the foregoing step 6 was subjected to the same operations used in the step 3 of Example 1 to give an intended compound.

Yield: 355 mg

MS (ESI): m/z: 695 [M+2H]²⁺

### Example 61

### TAA(TrpOrnDab)₃ Hydrochloride

### Step 1: Condensation

The compound (1.0 g, 0.72 mM) obtained in the step 4 of Example 60 and 820 μL (4.8 mM) of DIEA were added to a solution obtained by dissolving, in 5 mL of DMF, 756 mg (2.4 mM) of Z-Dab(Boc)-OH, 982 mg (2.4 mM) of HCTU and 402 mg (2.4 mM) of Cl-HOBt and the resulting mixture was stirred at room temperature overnight. After the completion of the reaction, the reaction liquid was diluted with ethyl acetate, washed with, in order, water, an aqueous saturated sodium bicarbonate solution, and an aqueous saturated common salt solution, dried over magnesium sulfate and then the solvent was distilled off. The resulting solids were washed with a mixed ethyl acetate/ether solvent. The solids were dried under a reduced pressure to give an intermediate.

Yield: Quantitative

### Step 2: Deprotection

Trifluoroacetic acid (5 mL) was added to the compound prepared in the foregoing step 1 and the resulting mixture was stirred at room temperature for one hour. The solvent was distilled off under a reduced pressure, followed by the addition of water to the resulting residue and the lyophilization of the aqueous mixture to thus remove the trifluoroacetic acid.

### Step 3: Ion-Exchanging and Conversion into Hydrochloride

The compound obtained in the foregoing step 2 was subjected to the same operations used in the step 3 of Example 1 to give an intended compound.

Yield: 389 mg

MS (ESI): m/z: 674 [M+2H]²⁺

### Example 62

### TETA(4-NH₂Bz)₄(Orn)₄(Orn)₈ Hydrochloride

### Step 1: Condensation and Reduction

4-Nitrobenzoyl chloride (5.5 g, 29.5 mM) was dropwise added to a solution of 1.0 mL (6.7 mM) of triethylene tetramine and 4.1 mL (29.5 mM) of TEA in 50 mL of dichloromethane and the mixture was stirred overnight. The solids separated out from the mixture were filtered off, suspended in 100 mL of methanol, followed by the addition of 2 g of 10% Pd/C and the subsequent stirring of the resulting mixture at room temperature for 5 hours in a hydrogen gas atmosphere. The reaction liquid was filtered through cerite and the solvent was distilled off to give an intermediate.

Amount of the intermediate obtained: 3.58 g (5.76 mM) (Yield: 86%)

### Step 2: Condensation and Deprotection

The compound (2.77 g, 4.45 mM) obtained in the foregoing step 1 and 5.4 mL (38.8 mM) of TEA were added to a solution prepared by dissolving, in 50 mL of DMF, 7.2 g (14.6 mM) of Z-Orn(Boc)-OH, 7.4 g (14.6 mM) of HATU and 2.7 g (14.6 mM) of HOAt, and the resulting mixture was stirred at room temperature overnight. After the completion of the reaction, the same post-treatment used in the step 1 of Example 1 was repeated, then 1.5 g of 10% Pd/C and 60 mL of methanol were added to the resulting compound and the mixture was stirred at room temperature overnight in a hydrogen gas atmosphere. The reaction liquid was filtered through cerite, the solvent was distilled off, the resulting residue was purified by the reversed phase high-performance liquid chromatography and the fraction containing an intended product was lyophilized to thus give an intermediate.

Amount of the intermediate obtained: 5.44 g (2.81 mM) (Yield: 63%)

### Step 3: Deprotection, Condensation and Deprotection

The compound (5.44 g, 2.81 mM) obtained in the foregoing step 2 was dissolved in 5 mL of trifluoroacetic acid, followed by the stirring of the solution for one hour and the removal of the solvent through distillation. A solution obtained by dissolving, in 15 mL of DMF, the compound obtained above and 6.7 mL (48 mM) of TEA was added to a solution prepared by dissolving, in 17 mL of DMF, 7.78 g (23.4 mM) of Boc-Orn(Boc)-OH, 8.9 g (23.4 mM) of HBTU and 3.6 g (23.4 mM) of HOBt and the resulting mixture was stirred at room temperature overnight. After the completion of the reaction, the same post-treatments used in the step 1 of Example 1 were repeated, the resulting residue was dissolved in 5 mL of trifluoroacetic acid and then the solution was stirred for one hour. The solvent was distilled off and the resulting residue was purified by the reversed phase high-performance liquid chromatography, followed by the lyophilization of the fraction obtained to thus give an intended compound.

Amount of the compound obtained: 1.03 g (0.27 mM) (Yield: 10%)

### Step 3: Ion-Exchanging and Conversion into Hydrochloride

The compound (1.03 g, 0.27 mM) obtained in the foregoing step 3 was subjected to the same operations used in the step 3 of Example 1 to give an intended compound.

Yield: 700 mg

### Example 63

### TETA(PheDab)₄ Hydrochloride

### Step 1: Condensation

There were dissolved, in 10 mL of DMF, 700 mg (2.2 mM) of Boc-Dab(Boc)OH, 843 mg (2.2 mM) of HBTU and 337 mg (2.2 mM) of HOBt. To this solution, there were added 367 mg (0.5 mM) of the compound prepared in the step 2 of Example 40: TETA(Phe)₄ and 460 μL (3.3 mM) of TEA and the resulting mixture was stirred at room temperature through the night. After the completion of the reaction, the same post-treatments used in the step 1 of Example 1 were repeated and the resulting product was used in the subsequent step without isolating and further purifying the same.

### Step 2: Deprotection

The residue obtained in the foregoing step 1 was dissolved in 5 mL of trifluoroacetic acid and the solution was stirred for one hour. The solvent was distilled off and the resulting residue was purified by the reversed phase high-performance liquid chromatography, followed by the lyophilization of the fraction obtained to thus give an intended compound.

Amount of the compound obtained: 528 mg (0.25 mM) (Yield: 51%)

MS (ESI): m/z: 568 [M+2H]²⁺

### Step 3: Ion-Exchanging and Conversion into Hydrochloride

The compound (528 mg, 0.25 mM) obtained in the foregoing step 2 was subjected to the same operations used in the step 3 of Example 1 to give an intended compound.

Yield: 319 mg

¹H-NMR (D₂O): δ: 1.95-2.4 (m, 8H), 2.6-4.0 (m, 36H), 7.0-7.4 (m, 20H)

### Example 64

### TEPA(PheOrn)₅ Hydrochloride

### Step 1: Condensation

To a solution of 10.06 (25.38 mM) of Z-Phe-OSu in 50 mL of dichloromethane, there was added 2.41 mL (12.69 mM) of tetraethylene pentamine, the mixture was stirred at room temperature through the night and then the solvent was distilled off under a reduced pressure. There were separately dissolved, in 50 mL of DMF, 13.29 g (44.4 mM) of Z-Phe-OH, 16.9 g (44.4 mM) of HATU and 6.0 g (44.4 mM) of HOAt. To this solution, there was added the residue obtained above after dissolving in 12.2 mL (88 mM) of TEA and 50 mL of DMF, and then the resulting mixture was stirred overnight. Ethyl acetate was added to the mixture, followed by the washing of the resulting mixture with, in order, water, a 0.2M aqueous solution of sodium hydroxide and a saturated aqueous common salt solution and the subsequent drying over magnesium sulfate. The solvent was distilled off under a reduced pressure, the resulting residue was purified by the reversed phase high-performance liquid chromatography, the fraction containing an intended product was lyophilized to give a compound, 2 g of 10% Pd/C and 100 mL of methanol were added to the resulting compound and the mixture was stirred at room temperature overnight in a hydrogen gas atmosphere. The reaction liquid was filtered through cerite and the solvent was distilled off to give an intermediate.

Amount of the intermediate obtained: 4.5 g (4.87 mM) (Yield: 38%)

### Step 2: Condensation

There were dissolved, in 12 mL of DMF, 914 mg (2.75 mM) of Boc-Orn(Boc)OH, 1043 mg (2.75 mM) of HBTU and 421 mg (2.75 mM) of HOBt. To this solution, there were added 462 mg (0.5 mM) of TEPA(Phe)₅, and 5754 (4.12 mM) of TEA and the resulting mixture was stirred at room temperature through the night. After the completion of the reaction, the same post-treatments used in the step 1 of Example 1 were repeated and the product was used in the subsequent reaction without isolating and purifying the same.

MS (ESI): m/z: 833 [M+3H]³⁺

### Step 3: Deprotection

The residue obtained in the foregoing step 2 was dissolved in 5 mL of trifluoroacetic acid and the solution was stirred for one hour. The solvent was distilled off, the resulting residue was purified by the reversed phase high-performance liquid chromatography, and the fraction containing an intended product was lyophilized to thus give an intermediate.

Amount of the intermediate obtained: 727 mg (0.27 mM) (Yield: 55%)

MS (ESI): m/z: 749 [M+2H]²⁺

### Step 4: Ion-Exchanging and Conversion into Hydrochloride

The compound (727 mg, 0.27 mM) obtained in the foregoing step 3 was subjected to the same operations used in the step 3 of Example 1 to give an intended compound.

Yield: 482 mg

¹H-NMR (D₂O): δ: 1.4-1.85 (m, 20H), 1.9-3.9 (m, 46H), 7.0-7.4 (m, 25H)

### Example 65

### TETA(OrnOrnOrn)₂(ProOrnOrn)₂ Hydrochloride

### Step 1: Condensation

To a mixture comprising 2.29 g (6.26 mM) of Z-Orn(Boc)OH and 1.10 g (6.26 mM) of CDI, there was added 30 mL of dichloromethane and the mixture was stirred to thus dissolve the components. To this solution, there was added 750 mg (5.13 mM) of triethylene tetramine and the resulting mixture was stirred at room temperature overnight. After the completion of the reaction, ethyl acetate and an aqueous 1 N sodium hydroxide solution were added to the reaction system and the system was thus extracted with ethyl acetate. The extract was washed with a saturated aqueous common salt solution, dried over sodium sulfate, followed by the filtration to thus give a compound.

Amount of the compound obtained: 2.15 g (2.55 mM) (Yield: 49.7%)

MS (ESI): m/z: 843 [M+2H]²⁺

### Step 2: Condensation

To a mixture containing 900 mg (3.61 mM) of Z-Pro-OH, 1.51 g (3.61 mM) of HATU and 541 mg (3.61 mM) of HOAt, there was added 18 mL of DMF and the mixture was stirred to thus dissolve the components. To this solution, there were added 2.15 g (2.55 mM) of the compound obtained in the foregoing step 1 and 760 μL (5.42 mM) of TEA and the mixture was stirred at room temperature overnight. After the completion of the reaction, the same post-treatment used in the step 1 of Example 1 was repeated to give an intermediate.

Amount of the intermediate obtained: 1.14 g (0.873 mM) (Yield: 48.3%)

MS (ESI): m/z: 653 [M+2H]²⁺

### Step 3: Deprotection

There were added, to the compound obtained in the foregoing step 2, 710 mg of 10% Pd/C and 10 mL of ethanol and the resulting mixture was stirred at room temperature overnight in a hydrogen gas atmosphere. The reaction liquid was filtered through cerite and then the solvent was distilled off to give a compound.

Amount of the compound obtained: 560 mg (0.728 mM) (Yield: 83.4%)

MS (ESI): m/z: 385 [M+2H]²⁺

### Step 4: Condensation

To a mixture comprising 1.07 g (2.92 mM) of Z-Orn(Boc)-OH, 1.16 g (2.92 mM) of HATU and 417 mg (2.92 mM) of HOAt, there was added 10 mL of DMF and the mixture was stirred to thus dissolve the components. To this solution, there were added 560 mg (0.728 mM) of the compound obtained in the foregoing step 3 and 5104 (3.65 mM) of TEA and the mixture was stirred at room temperature overnight. After the completion of the reaction, the same post-treatment used in the step 1 of Example 1 was repeated to give an intermediate.

Amount of the intermediate obtained: 1.48 g (0.683 mM) (Yield: 93.7%)

MS (ESI): m/z: 1081 [M+2H]²⁺

### Step 5: Deprotection

To the compound obtained in the foregoing step 4, there were added 700 mg of 10% Pd/C and 15 mL of ethanol and the resulting mixture was stirred at room temperature overnight in a hydrogen gas atmosphere. The reaction liquid was filtered through cerite and then the solvent was distilled off to give an intermediate.

Amount of the intermediate obtained: 1.24 g (0.760 mM) (Yield: quantitative)

MS (ESI): m/z: 813 [M+2H]²⁺

### Step 6: Condensation

To a mixture comprising 532.8 mg (1.45 mM) of Z-Orn(Boc)OH, 579.7 mg (1.52 mM) of HATU and 207.5 mg (1.52 mM) of HOAt, there was added 7 mL of DMF and the mixture was stirred to dissolve the components. To this solution, there were added 591 mg (0.363 mM) of the compound obtained in the foregoing step 5 and 253 μL (1.82 mM) of TEA and the resulting mixture was stirred at room temperature overnight. After the completion of the reaction, the same post-treatment used in the step 1 of Example 1 was repeated to give an intermediate.

Amount of the intermediate obtained: 779.6 mg (0.258 mM) (Yield: 71.2%)

MS (ESI): m/z: 1007 [M+3H]³⁺

### Step 7: Deprotection

To the compound obtained in the foregoing step 6, there were added 350 mg of 10% Pd/C and 15 mL of ethanol and the resulting mixture was stirred at room temperature overnight in a hydrogen gas atmosphere. The reaction liquid was filtered through cerite and then the solvent was distilled off to give a compound.

MS (ESI): m/z: 828 [M+H]⁺

### Step 8: Deprotection

Trifluoroacetic acid (6 mL) was added to the compound obtained in the foregoing step 7 and the resulting mixture was stirred at room temperature for one hour. The solvent was distilled off under a reduced pressure, the resulting residue was purified by the reversed phase high-performance liquid chromatography and the fraction was lyophilized to thus give an intended compound.

MS (ESI): m/z: 494 [M+3H]³⁺

### Step 9: Ion-Exchanging and Conversion into Hydrochloride

The compound obtained in the foregoing step 8 was subjected to the same operations used in the step 3 of Example 1 to give an intended compound.

Amount of the compound obtained: 373 mg (0.206 mM) (Yield: 80.0%)

MS (ESI): m/z: 494 [M+3H]³⁺

### Example 66

### TETA(ProOrnOrn)₂(OrnOrnOrn)₂ Hydrochloride

### Step 1: Condensation

To a mixture comprising 1.47 g (6.00 mM) of Z-Pro-OH, 1.84 g (6.60 mM) of HATU and 898 mg (6.60 mM) of HOAt, there was added 3 mL of DMF and the resulting mixture was stirred to dissolve the components. To this solution, there were added 437 mg (3.00 mM) of TETA and 1.25 mL (9.00 mM) of TEA and the mixture was stirred at room temperature overnight. After the completion of the reaction, the same post-treatment used in the step 1 of Example 1 was repeated and the product thus obtained was used in the subsequent step without isolating and purifying the same.

MS (ESI): m/z: 609 [M+H]⁺

### Step 2: Condensation

To a mixture of 2.20 g (6.00 mM) of Z-Orn(Boc)-OH, 2.51 g (6.60 mM) of HATU and 898 mg (6.60 mM) of HOAt, there was added 30 mL of DMF and the resulting mixture was stirred to thus dissolve the components. To this solution, there were added 2.94 g of the compound obtained in the foregoing step 1 and 1.25 mL (9.00 mM) of TEA and the mixture was stirred at room temperature overnight. After the completion of the reaction, the same post-treatment used in the step 1 of Example 1 was repeated to give an intermediate.

Amount of the intermediate obtained: 1.40 g (0.108 mM) (Yield: 35.9%)

MS (ESI): m/z: 653 [M+2H]²⁺

### Step 3: Deprotection

To the compound obtained in the foregoing step 2, there were added 700 mg of 10% Pd/C and 10 mL of ethanol and the resulting mixture was stirred at room temperature overnight in a hydrogen gas atmosphere. The reaction liquid was filtered through cerite and then the solvent was distilled off to give a compound.

Amount of the compound obtained: 946 mg (Yield: Quantitative)

MS (ESI): m/z: 385 [M+2H]²⁺

### Step 4: Condensation

DMF (25 mL) was added to a mixture comprising 1.81 g (4.94 mM) of Z-Orn(Boc)-OH, 1.97 g (5.18 mM) of HATU and 705 mg (5.18 mM) of HOAt and the resulting mixture was stirred to dissolve the components. To this solution, there were added 948 mg (1.23 mM) of TETAPro₂[Orn(Boc)]₂ and 869 μL (6.17 mM) of TEA and the mixture was stirred at room temperature overnight. After the completion of the reaction, the same post-treatment used in the step 1 of Example 1 was repeated to give an intended compound.

Amount of the compound obtained: 1.82 g (0.842 mM) (Yield: 68.3%)

MS (ESI): m/z: 1082.0 [M+2H]²⁺

### Step 5: Deprotection

To the compound obtained in the foregoing step 4, there were added 900 mg of 10% Pd/C and 10 mL of ethanol and the resulting mixture was stirred at room temperature overnight in a hydrogen gas atmosphere. The reaction liquid was filtered through cerite and the solvent was distilled off to give a compound.

Amount of the compound obtained: 1.50 g (0.925 mM) (Yield: Quantitative)

MS (ESI): m/z: 813 [M+2H]²⁺

### Step 6: Condensation

DMF (7 mL) was added to a mixture comprising 616 mg (1.85 mM) of Boc-Orn(Boc)-OH, 737 mg (1.95 mM) of HBTU and 265 mg (1.95 mM) of HOAt and the resulting mixture was stirred to dissolve the components. To this solution, there were added 753 mg (0.463 mM) of the compound obtained in the foregoing step 5 and 320 μL (2.32 mM) of TEA and the mixture was stirred at room temperature overnight. After the completion of the reaction, the same post-treatment used in the step 1 of Example 1 was repeated to give an intermediate.

MS (ESI): m/z: 962 [M+3H]³⁺

### Step 7: Deprotection

Trifluoroacetic acid (7 mM) was added to the compound obtained in the foregoing step 6 and the mixture was stirred at room temperature for one hour. The solvent was distilled off under a reduced pressure and the resulting residue was purified by the reversed phase high-performance liquid chromatography and the fraction containing an intended product was lyophilized to thus give an intermediate.

Yield: 1.63 g

MS (ESI): m/z: 494 [M+3H]³⁺

### Step 8: Ion-Exchanging and Conversion into Hydrochloride

The compound obtained in the foregoing step 2 was subjected to the same operations used in the step 3 of Example 1 to give an intended compound.

Amount of the compound obtained: 623 mg (0.347 mM) (Yield: 75.0%)

MS (ESI): m/z: 494 [M+3H]³⁺

### Example 67

### TETA(PheOrnDab)₄ Hydrochloride

### Step 1: Condensation

DMF (15 mL) was added to a mixture comprising 1.50 g (4.09 mM) of X-Orn(Boc)-OH, 1.63 g (4.29 mM) of HBTU and 657 mg (4.29 mM) of HOBt/H₂O and the resulting mixture was stirred to dissolve the components. To this solution, there were added 750 mg (1.02 mM) of TETA(Phe)₄ and 710 μL (5.11 mM) of TEA and the mixture was stirred at room temperature overnight. After the completion of the reaction, the same post-treatment used in the step 1 of Example 1 was repeated to give an intermediate.

MS (ESI): m/z: 1065 [M+2H]²⁺

### Step 2: Deprotection

To the compound obtained in the foregoing step 1, there were added 892 mg of 10% Pd/C and 10 mL of ethanol and the resulting mixture was stirred at room temperature overnight in a hydrogen gas atmosphere. The reaction liquid was filtered through cerite and the solvent was distilled off to give a compound.

Amount of the compound obtained: 1.63 g (1.03 mM) (Yield: Quantitative)

MS (ESI): m/z: 796 [M+2H]²⁺

### Step 3: Condensation

DMF (6 mL) was added to a mixture comprising 400 mg (1.26 mM) of Boc-Orn(Boc)-OH, 500 mg (1.32 mM) of HBTU and 202 mg (1.32 mM) of HOBt/H₂O and the resulting mixture was stirred to dissolve the components. To this solution, there were added 500 mg (0.314 mM) of the compound obtained in the foregoing step 2 and 220 μL (1.57 mM) of TEA and the resulting mixture was stirred at room temperature overnight. After the completion of the reaction, the same post-treatment used in the step 1 of Example 1 was repeated to give an intermediate.

### Step 4: Deprotection

Trifluoroacetic acid (6 mM) was added to the compound obtained in the foregoing step 3 and the mixture was stirred at room temperature for one hour. The solvent was distilled off under a reduced pressure, the resulting residue was purified by the reversed phase high-performance liquid chromatography and the fraction containing an intended product was lyophilized to thus give an intermediate.

Yield: 517 mg

MS (ESI): m/z: 797 [M+2H]²⁺

### Step 5: Ion-Exchanging and Conversion into Hydrochloride

The compound obtained in the foregoing step 4 was subjected to the same operations used in the step 3 of Example 1 to give an intended compound.

Amount of the compound obtained: 315 mg (0.162 mM)

MS (ESI): m/z: 797 [M+2H]²⁺

### Example 68

### TETA(PheOrn(3-Ala)₄ Hydrochloride

### Step 1: Condensation

DMF (6 mL) was added to a mixture of 500 mg (0.314 mM) of the compound: TETA[PheOrn(Boc)]₄ obtained in the step 4 of Example 40, 360 mg (1.26 mM) of Boc-β-AlaOSu and 3504 (2.51 mM) of TEA and the resulting mixture was stirred to thus dissolve the components. To this solution, there was added 750 mg (1.02 mM) of the compound: TETA(Phe)₄ obtained in the step 2 of Example 40 and the resulting mixture was stirred at room temperature overnight. After the completion of the reaction, the same post-treatment used in the step 1 of Example 1 was repeated to give an intended compound.

### Step 2: Deprotection

Trifluoroacetic acid (6 mM) was added to the compound obtained in the foregoing step 1 and the mixture was stirred at room temperature for one hour. The solvent was distilled off under a reduced pressure, the resulting residue was purified by the reversed phase high-performance liquid chromatography and the corresponding fraction was then lyophilized to thus give a compound.

Yield: 445.1 mg

MS (ESI): m/z: 738.0 [M+2H]²⁺

### Step 3: Ion-Exchanging and Conversion into Hydrochloride

The compound obtained in the foregoing step 2 was subjected to the same operations used in the step 3 of Example 1 to give an intended compound.

Amount of the compound obtained: 256 mg (0.142 mM)

MS (ESI): m/z: 738.0 [M+2H]²⁺

### Example 69

### TETA(OrnPheDab)₄ Hydrochloride

### Step 1: Condensation and Deprotection

CDI (1.86 g, 11.5 mM) was added to a solution of 4.21 g (11.5 mM) of Z-Orn(Boc)-OH in 30 mL of dichloromethane and the mixture was stirred at room temperature for 30 minutes. To the reaction solution, there was added 0.8 g (5.47 mM) of triethylene tetramine and the mixture was stirred at that temperature through the night. Then the solvent was distilled off under a reduced pressure, ethyl acetate was added to the resulting residue, the mixture was washed with, in order, water, a 0.2M aqueous sodium hydroxide solution and a saturated aqueous common salt solution and then dried over magnesium sulfate. The solvent was distilled off under a reduced pressure, 30 mL of DMF was added to the residue, followed by the addition of, in order, 4 g (10.9 mM) of Z-Orn(Boc)-OH, 4.16 g (10.9 mM) of HATU, 1.49 g (10.9 mM) of HOAt and 2.3 mL (16.5 mM) of TEA and the stirring of the resulting mixture at room temperature through the night. Ethyl acetate was added to the reaction solution, the mixture was washed with, in order, water, a 0.2M aqueous hydrochloric acid solution, a 0.2M aqueous sodium hydroxide solution and a saturated aqueous common salt solution, dried over magnesium sulfate and the solvent was distilled off under a reduced pressure. There were then added, to the resulting residue, 50 mL of methanol and 0.6 g of 10% Pd/C and the mixture was stirred through the night in a hydrogen gas atmosphere. The reaction liquid was filtered through cerite, the solvent was then distilled off under a reduced pressure, the resulting residue was purified by the reversed phase high-performance liquid chromatography and the fraction containing an intended product was lyophilized to thus give an intermediate.

Amount of the intermediate obtained: 4.84 mg (3.49 mM) (Yield: 63.6%)

MS (ESI): m/z: 502 [M+2H]²⁺

### Step 2: Condensation and Deprotection

To a solution of the compound (3.45 g, 2.49 mM) obtained in the foregoing step 1 in 40 mL of DMF, there were added, in order, 3.13 g (10.4 mM) of Z-Phe-OH, 3.96 g (10.4 mM) of HBTU, 1.6 g (10.4 mM) of HOBt.H₂O and 3.47 mL (24.9 mM) of TEA and the mixture was stirred at room temperature through the night. Ethyl acetate was added to the reaction solution, the mixture was washed with, in order, water, a 0.2M aqueous hydrochloric acid solution, a 0.2M aqueous sodium hydroxide solution and a saturated aqueous common salt solution, dried over magnesium sulfate and the solvent was distilled off under a reduced pressure. To the resulting residue, there were added 50 mL of methanol and 0.4 g of 10% Pd/C and the mixture was stirred at room temperature through the night in a hydrogen gas atmosphere. The reaction liquid was filtered through cerite, the solvent was distilled off under a reduced pressure, the resulting residue was purified by the reversed phase high-performance liquid chromatography and the fraction containing an intended product was lyophilized to thus give an intermediate.

Amount of the intermediate obtained: 3.83 g (1.94 mM) (Yield: 77.9%)

MS (ESI): m/z: 796 [M+2H]²⁺

### Step 3: Condensation and Deprotection

To a solution of the compound (2.0 g, 1.01 mM) obtained in the foregoing step 2 in 10 mL of DMF, there were added, in order, 1.36 g (4.27 mM) of Boc-Dab(Boc)-OH, 1.61 g (4.24 mM) of HBTU, 0.65 g ((4.24 mM) of HOBt.H₂O and 1.41 mL (10.2 mM) of TEA and the mixture was stirred at room temperature through the night. The same post-treatment used in the step 1 of Example 1 was then repeated to give an intermediate. Trifluoroacetic acid (6 mL) was added to the intermediate and the mixture was stirred at room temperature for one hour. The solvent was distilled off under a reduced pressure, the resulting residue was purified by the reversed phase high-performance liquid chromatography and the corresponding fraction was lyophilized to thus give an intermediate.

MS (ESI): m/z: 531 [M+3H]³⁺

### Step 4: Desalting and Conversion into Hydrochloride

After replacing Amberlite IRA910 as an anion-exchange resin with a 1 N aqueous sodium hydroxide solution, then the resin was replaced with water treated with a Millipore filter (hereunder referred to as “Millipore-water”) till the wash liquid became neutral. An aqueous solution of the compound obtained in the foregoing step 3 was loaded to the resin and Millipore-water was passed through the resin till the wash liquid became neutral. The solvent was partially distilled off under a reduced pressure to thus reduce the amount of the solution, the solution was lyophilized, then hydrochloric acid was added in such an amount that the concentration of the resulting hydrochloride was 18% by mass and then the solution was again lyophilized to give an intended product.

Amount of the product obtained: 695 mg (0.358 mM) (Yield: 35% calculated for the 4 steps)

MS (ESI): m/z: 531 [M+3H]³⁺

### Example 70

### TETA(OrnPheOrn)₄ Hydrochloride

### Step 1: Condensation and Deprotection

To a solution of the compound (1.9 g, 0.963 mM) obtained in the step 2 of Example 69 in DMF (10 mL), there were added, in order, 1.34 g (4.03 mM) of Boc-Orn(Boc)-OH, 1.53 g (4.03 mM) of HBTU, 0.62 g (4.05 mM) of HOBt.H₂O and 1.34 mL (9.61 mM) of TEA and the mixture was stirred at room temperature through the night. Ethyl acetate was added to the reaction solution, the mixture was washed with, in order, water, a 0.2M aqueous hydrochloric acid solution, a 0.2M aqueous sodium hydroxide solution and a saturated aqueous common salt solution, dried over magnesium sulfate and the solvent was distilled off under a reduced pressure. To the resulting residue, there was added trifluoroacetic acid, the resulting mixture was stirred at room temperature for one hour, the solvent was distilled off under a reduced pressure, the resulting residue was purified by the reversed phase high-performance liquid chromatography and the fraction containing an intended product was lyophilized to thus give an intermediate.

MS (ESI): m/z: 550 [M+3H]³⁺

### Step 2: Ion-Exchanging and Conversion into Hydrochloride

The compound obtained in step 1 was subjected to the same operations used in the step 3 of Example 1 to give an intended compound.

Amount of the compound obtained: 703 mg (0.350 mM) (Yield: 36% calculated for the 2 steps)

MS (ESI): m/z: 550 [M+3H]³⁺

### Example 71

### TAA[PheDab(Dab)₂]₃ Hydrochloride

### Step 1: Condensation and Deprotection

CDI (2.51 g, 15.5 mM) was added to a solution of Z-Phe-OH (4.64 g, 15.5 mM) in dichloromethane (50 mL), the resulting mixture was stirred at room temperature for 30 minutes. To the reaction solution, there was added 0.73 g (4.99 mM) of tris(2-aminoethyl)-amine, the mixture was stirred at that temperature through the night, the solvent was distilled off under a reduced pressure, ethyl acetate was added to the resulting residue, the mixture was washed with, in order, water, a 0.2M aqueous sodium hydroxide solution and a saturated aqueous common salt solution and dried over magnesium sulfate. The solvent was distilled off under a reduced pressure, there were added, to the resulting residue, 100 mL of methanol, 30 mL of THF and 0.3 g of 10% Pd/C and the mixture was stirred through the night in a hydrogen gas atmosphere. The reaction liquid was filtered through cerite to give crude crystals of an Intermediate.

MS (ESI): m/z: 588 [M+H]⁺

### Step 2: Condensation and Deprotection

To a solution of the crude crystals (2.0 g, 3.41 mM) obtained in the foregoing step 1 in DMF (15 mL), there were added, in order, 3.25 g (10.2 mM) of Boc-Orn(Boc)-OH, 3.88 g (10.2 mM) of HBTU, 1.56 g (10.2 mM) of HOBt.H₂O and 1.9 mL (13.6 mM) of TEA and the mixture was stirred at room temperature through the night. Ethyl acetate was added to the reaction solution, the mixture was washed with, in order, water, a 0.2M aqueous hydrochloric acid solution, a 0.2M aqueous sodium hydroxide solution and a saturated aqueous common salt solution, dried over magnesium sulfate and the solvent was distilled off under a reduced pressure. To the resulting residue, there was added trifluoroacetic acid, the resulting mixture was stirred at room temperature for one hour, the solvent was distilled off under a reduced pressure, the resulting residue was purified by the reversed phase high-performance liquid chromatography and the fraction containing an intended product was lyophilized to thus give an intermediate.

Amount of the intermediate obtained: 3.84 g (2.62 mM) (Yield: 77%)

MS (ESI): m/z: 445 [M+2H]²⁺

### Step 3: Condensation and Deprotection

To a solution of the compound (1.5 g, 1.03 mM) obtained in the foregoing step 2 in DMF (20 mL), there were added, in order, 2.0 g (6.28 mM) of Boc-Orn(Boc)-OH, 2.4 g (6.33 mM) of HBTU, 0.97 g (6.33 mM) of HOBt.H₂O and 2.14 mL (15.4 mM) of TEA and the mixture was stirred at room temperature through the night. Ethyl acetate was added to the reaction solution, the mixture was washed with, in order, water, a 0.2M aqueous hydrochloric acid solution, a 0.2M aqueous sodium hydroxide solution and a saturated aqueous common salt solution, dried over magnesium sulfate and the solvent was distilled off under a reduced pressure. To the resulting residue, there was added trifluoroacetic acid, the resulting mixture was stirred at room temperature for one hour, the solvent was distilled off under a reduced pressure, the resulting residue was purified by the reversed phase high-performance liquid chromatography and the fraction containing an intended product was lyophilized to thus give an intermediate.

MS (ESI): m/z: 497 [M+3H]³⁺

### Step 4: Ion-Exchanging and Conversion into Hydrochloride

The compound obtained in the foregoing step 3 was subjected to the same operations used in the step 3 of Example 1 to thus give an intended compound.

Amount of the compound obtained: 620 mg (0.342 mM) (Yield: 33%, calculated for the 4 steps)

MS (ESI): m/z: 497 [M+3H]³⁺

### Example 72

### TAA[3,5-NH₂Bz(Orn)₂]₃ Hydrochloride

### Step 1: Condensation

CDI (5.86 g, 36.1 mM) was added to a solution of Boc-Orn(Boc)-OH (11.47 g, 34.5 mM) in THF (100 mL). After stirring the mixture at room temperature for 30 minutes, there were added, to the mixture, in order, 2.5 g (16.4 mM) of 3,5-diamino-benzoic acid and 5.04 mL (36.1 mM) of TEA and the resulting mixture was stirred at 60□ through the night. After cooling the same to room temperature, the solvent was distilled off under a reduced pressure. Ethyl acetate was added to the resulting residue, the mixture was washed with, in order, water, a 0.2M aqueous hydrochloric acid solution, a 0.2M aqueous sodium hydroxide solution and a saturated aqueous common salt solution, and then dried over magnesium sulfate. The solvent was then distilled off under a reduced pressure, the resulting residue was subjected to the slurry-washing with ethyl acetate, diethyl ether and hexane to thus give an intermediate.

Amount of the intermediate obtained: 11.0 g (14.1 mM) (Yield: 86%)

MS (ESI): m/z: 781 [M+H]⁺

### Step 2: Condensation and Deprotection

To a solution of the compound (8.0 g, 10.3 mM) obtained in the foregoing step 1 and tris(2-aminoethyl)-amine (0.49 mL, 3.27 mM) in DMF (40 mL), there were added 4.3 g (11.3 mM) of HATU, 1.5 g (11.0 mM) of HOAt and 1.57 mL (11.3 mM) of TEA and the resulting mixture was stirred at room temperature through the night. Ethyl acetate was added to the reaction solution, the mixture was washed with, in order, water, a 0.2M aqueous hydrochloric acid solution, a 0.2M aqueous sodium hydroxide solution and a saturated aqueous common salt solution, then dried over magnesium sulfate and the solvent was distilled off under a reduced pressure. Trifluoroacetic acid was added to the resulting residue, the resulting mixture was stirred at room temperature for one hour, the solvent was distilled off under a reduced pressure, the resulting residue was purified by the reversed phase high-performance liquid chromatography and the fraction containing an intended product was lyophilized to thus give an intermediate.

MS (ESI): m/z: 412 [M+3H]³⁺

### Step 3: Ion-Exchanging and Conversion into Hydrochloride

The compound obtained in the foregoing step 2 was subjected to the same operations used in the step 3 of Example 1 to thus give an intended compound.

Amount of the compound obtained: 1.84 mg (1.22 mM) (Yield: 37%, calculated for the 3 steps

MS (ESI): m/z: 412 [M+3H]³⁺

### Example 73

### TETA(OrnOrn)₂(LeuOrn)₂ Hydrochloride

### Step 1: Condensation

CDI (2.51 g, 15.5 mM) was added to a solution of Z-Orn(Boc)-OH (5.1 g, 15.5 mM) in dichloromethane (50 mL) and the mixture was stirred at room temperature for 30 minutes. Triethylene tetramine (1.13 g, 7.75 mM) was added to the reaction solution, the mixture was stirred at that temperature through the night, the solvent was distilled off under a reduced pressure, then ethyl acetate was added to the resulting residue, the mixture was washed with, in order, water, a 0.2M aqueous sodium hydroxide solution and a saturated aqueous common salt solution and then dried over magnesium sulfate.

### Step 2: Condensation

There were dissolved, in 12 mL of DMF, 1.46 g (5.5 mM) of Z-Leu-OH, 2.08 g (5.5 mM) of HATU and 6.2 g (5.5 mM) of HOAt. To this solution, there were added 2.1 g (2.5 mM) of TETA[Z-Orn(Boc)]₂ synthesized in the foregoing step 1 and 1150 μL (8.25 mM) of TEA and the resulting mixture was stirred at room temperature through the night. After the completion of the reaction, the same post-treatment used in the step 1 of Example 1 was repeated and the product thus obtained was used in the subsequent step without isolating and purifying the same.

MS (ESI): m/z: 669 [M+2H]²⁺

### Step 3: Deprotection

To the residue obtained in the foregoing step 2, there was added 350 mg of 10% Pd/C and the mixture was dissolved and dispersed in 8 mL of methanol. After replacing the atmosphere of the reaction system with hydrogen, the system was stirred at room temperature overnight. After the removal of the Pd/C through filtration, the solvent was distilled off from the filtrate, the resulting residue was purified by the reversed phase high-performance liquid chromatography and the fraction containing an intended product was lyophilized to thus give an intermediate.

Amount of the intermediate obtained: 1.47 g (1.17 mM) (Yield: 46%, calculated for the two steps)

MS (ESI): m/z: 801 [M+H]^{±}

### Step 4: Condensation

There were dissolved, in 15 mL of DMF, 1.88 g (5.15 mM) of Z-Orn(Boc)-OH, 1.95 g (5.15 mM) of HBTU and 789 mg (5.15 mM) of HOBt. To this solution, there were added 1.47 g (1.17 mM) of the compound obtained in the foregoing step 2 and 1077 μL (7.73 mM) of TEA and the resulting mixture was stirred at room temperature through the night. After the completion of the reaction, the same post-treatment used in the step 1 of Example 1 was repeated and the product thus obtained was used in the subsequent step without isolating and purifying the same.

MS (ESI): m/z: 732 [M+3H]³⁺

### Step 5: Deprotection

To the residue obtained in the foregoing step 4, there was added 300 mg of 10% Pd/C and the mixture was dissolved and dispersed in 15 mL of methanol. After replacing the atmosphere of the reaction system with hydrogen, the system was stirred at room temperature overnight. After the removal of the Pd/C through filtration, the solvent was distilled off from the filtrate, the resulting residue was purified by the reversed phase high-performance liquid chromatography and the fraction containing an intended product was lyophilized to thus give an intermediate.

Amount of the intermediate obtained: 1.34 g (0.63 mM) (Yield: 60%, calculated for the two steps)

MS (ESI): m/z: 829 [M+2H]²⁺

### Step 6: Deprotection

The compound (1.34 g, 0.63 mM) obtained in the foregoing step 5 was dissolved in 5 mL of trifluoroacetic acid and the solution was stirred for one hour. The solvent was distilled off, the resulting residue was purified by the reversed phase high-performance liquid chromatography, and the fraction containing an intended product was lyophilized to thus give an intermediate.

Amount of the intermediate obtained: 1.14 g (0.52 mM) (Yield: 83%)

MS (ESI): m/z: 529 [M+2H]²⁺

### Step 7: Ion-Exchanging and Conversion into Hydrochloride

The compound (1.14 g, 0.52 mM) obtained in the foregoing step 6 was subjected to the same operations used in the step 3 of Example 1 to give an intended compound.

Yield: 668 mg

¹H-NMR (D₂O): δ: 0.6-0.85 (m, 12H), 0.9-1.8 (m, 28H), 2.7-3.1 (m, 12H), 3.1-4.5 (m, 22H)

### Example 74

### TETA[PheAsp(DETA)]₄ Hydrochloride

### Step 1: Condensation and Deprotection

DMF (15 mL) was added to a mixture comprising 1.36 g (4.21 mM) of BocAsp(OBn)OH, 1.63 g (4.29 mM) of HBTU and 657 mg (4.29 mM) of HOBt/H₂O and the resulting mixture was stirred to dissolve the components. To this solution, there were added 750 mg (1.02 mM) of TETA(Phe)₄ and 710 μL (5.11 mM) of TEA and the mixture was stirred at room temperature overnight. After the completion of the reaction, the same post-treatment used in the step 1 of Example 1 was repeated, then 350 mg of 10% Pd/C was added to the resulting residue and the mixture was dissolved and dispersed in 18 mL of methanol. After replacing the atmosphere of the reaction system with hydrogen, the system was stirred at room temperature overnight. After the removal of the Pd/C through filtration, the solvent was distilled off from the filtrate, the resulting residue was purified by the reversed phase high-performance liquid chromatography and the fraction containing an intended product was lyophilized to thus give an intermediate.

Amount of the intermediate obtained: 970 mg (0.61 mM) (Yield: 14%, calculated for the two steps)

### Step 2: Condensation and Deprotection

DMF (15 mL) was added to a mixture comprising 970 mg (0.61 mM) of the compound obtained in the foregoing step 1, 1.0 g (2.68 mM) of HATU and 364 mg (2.68 mM) of HOAt and the resulting mixture was stirred to dissolve the components. To this solution, there were added 994 mg (2.68 mM) of 2-(2-benzyloxycarbonyl-aminoethylamino) ethyl) carbamic acid benzyl ester and 746 μL (5.11 mM) of TEA and the mixture was stirred at room temperature overnight. The solvent was distilled off from the filtrate, the resulting residue was purified by the reversed phase high-performance liquid chromatography and the fraction containing an intended product was lyophilized to thus give an intermediate.

### Step 3: Deprotection, Ion-Exchanging and Conversion into Hydrochloride

After trifluoroacetic acid was added to the compound obtained in the foregoing step 2 and the mixture was stirred at room temperature for one hour, the solvent was distilled off under a reduced pressure, the resulting residue was purified by the reversed phase high-performance liquid chromatography and the fraction containing an intended product was lyophilized to thus give an intermediate. The compound thus obtained was subjected to the same operations used in the step 3 of Example 1 to thus give an intended compound.

MS (ESI): m/z: 513 [M+3H]³⁺

### Example 75

### TETA(LeuOrn)₄ Hydrochloride

### Step 1: Condensation

There were dissolved, in 15 mL of dichloromethane, 5.3 g (20.0 mM) of Z-Leu-OH and 3.2 g (20.0 mM) of CDI and the solution was stirred at room temperature for one hour. To this solution, there was added 1433 μL (10.0 mM) of triethylene tetramine and the resulting mixture was stirred at room temperature through the night. After the completion of the reaction, the solvent was distilled off, the resulting residue was purified by the reversed phase high-performance liquid chromatography and the fraction containing an intended product was lyophilized to thus give and intermediate.

Amount of the intermediate obtained: 5.55 g (6.4 mM) (Yield: 64%)

MS (ESI): m/z: 641 [M+H]⁺

### Step 2: Condensation

There were dissolved, in 25 mL of DMF, 1.86 g (7.0 mM) of Z-Leu-OH, 2.66 g (7.0 mM) of HATU and 958 mg (7.0 mM) of HOAt. To this solution, there were added 2.78 g (3.2 mM) of the compound obtained in the foregoing step 1 and 1.47 mL (10.5 mM) of TEA and the resulting mixture was stirred at room temperature through the night. After the completion of the reaction, the same post-treatment used in the step 1 of Example 1 was repeated, followed by the purification according to the reversed phase high-performance liquid chromatography and the lyophilization of the fraction containing an intended product to thus give an intermediate.

Amount of the intermediate obtained: 2.32 g (3.96 mM) (Yield: 64%)

MS (ESI): m/z: 1136 [M+H]⁺

### Step 3: Deprotection

To the compound (2.32 g, 3.96 mM) obtained in the foregoing step 2, there was added 232 mg of 10% Pd/C and the mixture was dissolved and dispersed in 20 mL of methanol. After replacing the atmosphere of the reaction system with hydrogen, the system was stirred at room temperature overnight. After the removal of the Pd/C through filtration, the solvent was distilled off from the filtrate and the resulting product was directly used in the subsequent step without isolating and purifying the same.

MS (ESI): m/z: 599 [M+H]⁺

### Step 4: Condensation

There were dissolved, in 20 mL of DMF, 3.3 g (9.02 mM) of Z-Orn(Boc)-OH, 3.42 g (9.02 mM) of HBTU and 1.38 g (9.02 mM) of HOBt. To the resulting solution, there were added the residue obtained in the foregoing step 3 and 1.88 mL (13.5 mM) of TEA and the resulting mixture was stirred at room temperature through the night. After the completion of the reaction, the same post-treatment used in the step 1 of Example 1 was repeated, followed by the purification according to the reversed phase high-performance liquid chromatography and the lyophilization of the fraction containing an intended product to thus give an intermediate.

Amount of the intermediate obtained: 3.04 g (0.71 mM) (Yield: 74%)

MS (ESI): m/z: 996 [M+2H]²⁺

### Step 5: Deprotection

To the compound (3.04 g, 0.71 mM) obtained in the foregoing step 4, there was added 304 mg of 10% Pd/C and the mixture was dissolved and dispersed in 20 mL of methanol. After replacing the atmosphere of the reaction system with hydrogen, the system was stirred at room temperature overnight. After the removal of the Pd/C through filtration, the solvent was distilled off from the filtrate and the resulting product was directly used in the subsequent step without isolating and purifying the same.

MS (ESI): m/z: 728 [M+2H]²⁺

### Step 6: Deprotection

The compound obtained in the foregoing step 5 was dissolved in 10 mL of trifluoroacetic acid and the solution was stirred at room temperature for one hour. The solvent was distilled off, the resulting residue was purified by the reversed phase high-performance liquid chromatography, and the fraction containing an intended product was lyophilized to thus give an intermediate.

Amount of the intermediate obtained: 2.20 g (1.12 mM) (Yield: 74%, calculated for the two steps)

MS (ESI): m/z: 528 [M+2H]²⁺

### Step 7: Ion-Exchanging and Conversion into Hydrochloride

The compound (2.20 g, 1.12 mM) obtained in the foregoing step 6 was subjected to the same operations used in the step 3 of Example 1 to give an intended compound.

Yield: 1.46 g

¹H-NMR (D₂O): δ: 0.75-0.9 (m, 24H), 1-1.9 (m, 28H), 2.8-3.0 (m, 8H), 3.0-4.3 (m, 20H)

### Example 76

### TAA[(Benzoyl-4-methylene)DETA]₃ Hydrochloride

### Step 1: Condensation

There was added 3.2 g (16.5 mM) of 4-chloromethyl-benzoyl chloride to a solution prepared by dissolving, in 5 mL of DMF, 730 mg (5 mM) of tris(2-aminoethyl) amine and 4.20 mL (30 mM) of TEA at a temperature of 0□ and the resulting mixture was stirred at room temperature overnight. After the completion of the reaction, the reaction liquid was diluted with ethyl acetate, the diluted liquid was washed with, in order, water, an aqueous saturated ammonium chloride solution, an aqueous saturated sodium bicarbonate solution, and an aqueous saturated common salt solution, the liquid was then dried over magnesium sulfate and the solvent was distilled off. The resulting solid was washed with ether. The solid was dried under a reduced pressure to thus give an intermediate.

Amount of the intermediate obtained: 1.32 mg (2.19 mM) (Yield: 44%)

### Step 2: Condensation

To a solution obtained by dissolving 1.0 g (1.66 mM) of the compound obtained in the foregoing step 1 and 943 μL (5.47 mM) of DIEA, there was added 1.66 g (5.48 mM) of DETA(Boc)₂ synthesized according to a known technique (Organic Letters, 2000, 14(2), 2117) and the mixture was stirred at room temperature overnight. After the completion of the reaction, the reaction liquid was diluted with ethyl acetate, the diluted liquid was then washed with water, an aqueous saturated sodium bicarbonate solution and an aqueous saturated common salt solution, the liquid was then dried over magnesium sulfate and the solvent was distilled off. The resulting solid was washed with an ethyl acetate-ether mixed solvent. The solvent was distilled off under a reduced pressure, the resulting residue was purified by the reversed phase high-performance liquid chromatography and the fraction containing an intended product was lyophilized to thus give an intermediate.

### Step 3: Deprotection

Trifluoroacetic acid (5 mL) was added to the compound obtained in the foregoing step 2 and the mixture was stirred at room temperature for one hour. The solvent was distilled off under a reduced pressure, followed by the addition of water to the resulting residue and the lyophilization of the aqueous mixture to thus remove the excess trifluoroacetic acid.

### Step 4: Ion-Exchanging and Conversion into Hydrochloride

The compound obtained in the foregoing step 3 was subjected to the same operations used in the step 3 of Example 1 to give an intended compound.

Yield: 179 mg

MS (ESI): m/z: 402 [M+2H]²⁺

The structural formulas of the polymers synthesized in the foregoing Examples 1 to 76 will be summarized in the following Tables 1 to 8.

In this respect, however, the substituted positions of amino groups on spermine, spermidine, triethylene tetramine, tetraethylene pentamine and tris(2-aminoethyl) amine appearing in these Tables will be expressed as follows for the convenience of expression:

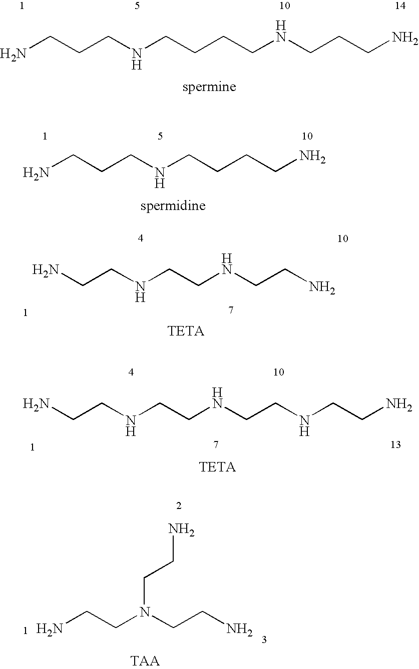

TABLE 1

| | | | | | | Spermine | | |
|---|---|---|---|---|---|---|---|---|
| Example | | | 1 | | | 5 | | |
| 1 | SPM(Lys)4 | 1st | | Lys | | | Lys | |
| 2 | SPM(Lys)12 | 1st | α | Lys | ε | α | Lys | ε |
| | | 2nd | Lys | | Lys | Lys | | Lys |
| 3 | SPM(Lys)4(Arg)8 | 1st | α | Lys | ε | α | Lys | ε |
| | | 2nd | Arg | | Arg | Arg | | Arg |
| 4 | SPM(Orn)4 | 1st | | Orn | | | Orn | |
| 5 | SPM(Arg)4 | 1st | | Arg | | | Arg | |
| 6 | SPM(Lys)6 | 1st | α | Lys | ε | α | Lys | ε |
| | | 2nd | | | | | | Lys |
| 7 | SPM(Lys)6 | 1st | α | Lys | ε | | | |
| | | 2nd | Lys | | Lys | | | |
| 8 | SPM(Orn)12 | 1st | α | Orn | δ | α | Orn | δ |
| | | 2nd | Orn | | Orn | Orn | | Orn |
| 9 | SPM(Orn)4 | 1st | α | Orn | δ | α | | δ |
| | | 2nd | Orn | | | | | |
| 10 | SPM(Lys)2(Orn)2 | 1st | | Lys | | | Orn | |
| 11 | SPM(Lys)8 | 1st | α | | ε | α | Lys | ε |
| | | 2nd | | | | Lys | | Lys |
| 12 | SPM(Lys)6 | 1st | | | | α | Lys | ε |
| | | 2nd | | | | Lys | | Lys |
| 13 | SPM(Lys)2(Orn)2 | 1st | | Orn | | | Lys | |
| 14 | SPM(Orn)8 | 1st | α | Orn | δ | α | Orn | δ |
| | | 2nd | | | Orn | | | Orn |
| 15 | SPM(Lys)8 | 1st | α | Lys | δ | α | Lys | ε |
| | | 2nd | | | Lys | | | Lys |
| Example | | | 10 | | | 14 | | |
| 1 | SPM(Lys)4 | 1st | | Lys | | | Lys | |
| 2 | SPM(Lys)12 | 1st | α | Lys | ε | α | Lys | ε |
| | | 2nd | Lys | | Lys | Lys | | Lys |
| 3 | SPM(Lys)4(Arg)8 | 1st | α | Lys | ε | α | Lys | ε |
| | | 2nd | Arg | | Arg | Arg | | Arg |
| 4 | SPM(Orn)4 | 1st | | Orn | | | Orn | |
| 5 | SPM(Arg)4 | 1st | | Arg | | | Arg | |
| 6 | SPM(Lys)6 | 1st | α | Lys | ε | α | Lys | ε |
| | | 2nd | | | Lys | | | |
| 7 | SPM(Lys)6 | 1st | | | | α | Lys | ε |
| | | 2nd | | | | Lys | | Lys |
| 8 | SPM(Orn)12 | 1st | α | Orn | δ | α | Orn | δ |
| | | 2nd | Orn | | Orn | Orn | | Orn |
| 9 | SPM(Orn)4 | 1st | | | | α | Orn | δ |
| | | 2nd | | | | Orn | | |
| 10 | SPM(Lys)2(Orn)2 | 1st | | Orn | | | Lys | |
| 11 | SPM(Lys)8 | 1st | α | Lys | ε | α | Lys | ε |
| | | 2nd | Lys | | Lys | Lys | | |
| 12 | SPM(Lys)6 | 1st | α | Lys | ε | | | |
| | | 2nd | Lys | | Lys | | | |
| 13 | SPM(Lys)2(Orn)2 | 1st | | Lys | | | Orn | |
| 14 | SPM(Orn)8 | 1st | α | Orn | δ | α | Orn | δ |
| | | 2nd | | | Orn | | | Orn |
| 15 | SPM(Lys)8 | 1st | α | Lys | ε | α | Lys | ε |
| | | 2nd | | | Lys | | | Lys |

TABLE 2

| | | | | | | | Spermine | | | |
|---|---|---|---|---|---|---|---|---|---|---|
| Example | | | 1 | | | | 5 | | | |
| 16 | SPM(Lys)8 | 1st | α | Lys | ε | | α | Lys | | ε |
| | | 2nd | Lys | | | | Lys | | | |
| 17 | SPM(Orn)2 | 1st | | Orn | | | | | | |
| 18 | SPM(Arg)2 | 1st | | Arg | | | | | | |
| 19 | SPM(Lys)2(Arg)2 | 1st | | Arg | | | | Lys | | |
| 20 | SPM(Lys)12 | 1st | α | Lys | ε | | α | Lys | | ε |
| | | 2nd | Lys | | | | Lys | | | |
| | | | α | ε | | | α | ε | | |
| | | 3rd | Lys | | | | Lys | | | |
| 21 | SPM(Gly)4 | 1st | | Gly | | | | Gly | | |
| 22 | SPM(Orn)8 | 1st | α | Orn | δ | | α | Orn | δ | |
| | | 2nd | Orn | | | | Orn | | | |
| 23 | SPM(Lys)2(Arg)2 | 1st | | Lys | | | | Arg | | |
| 24 | SPM(Arg)2 | 1st | | | | | | Arg | | |
| 25 | SPM(Gaa)4 | 1st | | Gaa | | | | Gaa | | |
| 26 | SPM(Ape)4 | 1st | | Ape | | | | Ape | | |
| 27 | SPM(Lys)9 | 1st | α | Lys | ε | | α | Lys | ε | |
| | | 2nd | Lys | | Lys | | Lys | | Lys | |
| 28 | SPM(Orn)12 | 1st | α | Orn | δ | | α | Orn | | δ |
| | | 2nd | Orn | | | | Orn | | | |
| | | | α | δ | | | α | δ | | |
| | | 3rd | Orn | | | | Orn | | | |
| 29 | SPM(Lys)12 | 1st | α | Lys | ε | | α | Lys | ε | |
| | | 2nd | | | Lys | | | | Lys | |
| | | | | | α | ε | | | α | ε |
| | | 3rd | | | Lys | | | | Lys | |
| 30 | SPM(Orn)2(Arg)2 | 1st | | Orn | | | | Arg | | |
| Example | | | 10 | | | | 14 | | | |
| 16 | SPM(Lys)8 | 1st | α | Lys | ε | | α | Lys | | ε |
| | | 2nd | Lys | | | | Lys | | | |
| 17 | SPM(Orn)2 | 1st | | | | | | Orn | | |
| 18 | SPM(Arg)2 | 1st | | | | | | Arg | | |
| 19 | SPM(Lys)2(Arg)2 | 1^{st} | | Lys | | | | Arg | | |
| 20 | SPM(Lys)12 | 1st | α | Lys | ε | | α | Lys | ε | |
| | | 2nd | Lys | | | | Lys | | | |
| | | | α | ε | | | α | ε | | |
| | | 3rd | Lys | | | | Lys | | | |
| 21 | SPM(Gly)4 | 1st | | Gly | | | | Gly | | |
| 22 | SPM(Orn)8 | 1st | α | Orn | δ | | α | Orn | δ | |
| | | 2nd | Orn | | | | Orn | | | |
| 23 | SPM(Lys)2(Arg)2 | 1st | | Arg | | | | Lys | | |
| 24 | SPM(Arg)2 | 1st | | Arg | | | | | | |
| 25 | SPM(Gaa)4 | 1st | | Gaa | | | | Gaa | | |
| 26 | SPM(Ape)4 | 1st | | Ape | | | | Ape | | |
| 27 | SPM(Lys)9 | 1st | α | Lys | ε | | | | | |
| | | 2nd | Lys | | Lys | | | | | |
| 28 | SPM(Orn)12 | 1st | α | Orn | δ | | α | Orn | δ | |
| | | 2nd | Orn | | | | Orn | | | |
| | | | α | δ | | | α | δ | | |
| | | 3rd | Orn | | | | Orn | | | |
| 29 | SPM(Lys)12 | 1st | α | Lys | ε | | α | Lys | ε | |
| | | 2nd | | | Lys | ε | | | Lys | |
| | | | | | α | | | | α | ε |
| | | 3rd | | | Lys | | | | Lys | |
| 30 | SPM(Orn)2(Arg)2 | 1st | | Arg | | | | Orn | | |

TABLE 3

SPD

| Example No. | | 1 | 5 | 10 |
|---|---|---|---|---|
| 31 | SPD (Orn)₃ | Orn | Orn | Orn |
| 32 | SPD (Arg)₃ | Arg | Arg | Arg |
| 33 | SPD (Orn)₂ | Orn | | Orn |

TABLE 4

| | | | TETA | | | | | |
|---|---|---|---|---|---|---|---|---|
| Example | | | 1 | | | 4 | | |
| 37 | TETA(PheOrn)2(OrnOrn)2 | 1st | | Phe | | | Orn | |
| | | 2nd | | α | | α | | δ |
| | | | | Orn | | Orn | | |
| 38 | TETA(Orn)2[Orn(Orn)2]2 | 1st | | Orn | | | Orn | |
| | | 2nd | α | | δ | α | | δ |
| | | | | | | Orn | | Orn |
| 39 | TETA[Orn(Orn)2]2(Orn)2 | 1st | | Orn | | | Orn | |
| | | 2nd | α | | δ | α | | δ |
| | | | Orn | | Orn | | | |
| 40 | TETA(Phe-δOrnOrn)4 | 1st | | Phe | | | Phe | |
| | | 2nd | | α | | | α | |
| | | | | Orn | | | Orn | |
| | | 3rd | α | δ | | α | δ | |
| | | | | Orn | | | Orn | |
| 41 | TETA(Orn)4[Dab(Dab)2]4 | 1st | | Orn | | | Orn | |
| | | 2nd | α | | δ | α | | δ |
| | | | Dab | | Dab | Dab | | Dab |
| 43 | TETA(PheOrn)2[Orn(Orn)2]2 | 1st | | Phe | | | Orn | |
| | | 2nd | | α | | α | | δ |
| | | | | Orn | | Orn | | Orn |
| 44 | TETA(PheOrn)2(δOrnOrn)2 | 1st | | Phe | | | Orn | |
| | | 2nd | | α | | α | | δ |
| | | | | Orn | | | | Orn |
| 46 | TETA(DPheOrn)2(OrnOrn)2 | 1st | | D-Phe | | | Orn | |
| | | 2nd | | α | | α | | δ |
| | | | | Orn | | Orn | | |
| 47 | TETA(TrpOrn)2(OrnOrn)2 | 1st | | Trp | | | Orn | |
| | | 2nd | | α | | α | | δ |
| | | | | Orn | | Orn | | |
| 49 | TETA(Phe-αOrnOrn)4 | 1st | | Phe | | | Phe | |
| | | 2nd | | α | | | α | |
| | | | | Orn | | | Orn | |
| | | 3rd | α | δ | | α | δ | |
| | | | Orn | | | Orn | | |
| 50 | TETA(OrnOrn)2(PheOrn)2 | 1st | | Orn | | | Phe | |
| | | 2nd | α | | δ | | α | |
| | | | Orn | | | | Orn | |
| 51 | TETA[Phe-Orn(Orn)2]4 | 1st | | Phe | | | Phe | |
| | | 2nd | | α | | | α | |
| | | | | Orn | | | Orn | |
| | | 3rd | α | δ | | α | δ | |
| | | | Orn | Orn | | Orn | Orn | |
| | | | TETA | | | | | |
| Example | | | 7 | | | 10 | | |
| 37 | TETA(PheOrn)2(OrnOrn)2 | 1st | | Orn | | | Phe | |
| | | 2nd | α | | δ | | α | |
| | | | Orn | | | | Orn | |
| 38 | TETA(Orn)2[Orn(Orn)2]2 | 1st | | Orn | | | Orn | |
| | | 2nd | α | | δ | α | | δ |
| | | | Orn | | Orn | | | |
| 39 | TETA[Orn(Orn)2]2(Orn)2 | 1st | | Orn | | | Orn | |
| | | 2nd | α | | δ | α | | δ |
| | | | | | | Orn | | Orn |
| 40 | TETA(Phe-δOrnOrn)4 | 1st | | Phe | | | Phe | |
| | | 2nd | | α | | | α | |
| | | | | Orn | | | Orn | |
| | | 3rd | α | δ | | α | δ | |
| | | | | Orn | | | Orn | |
| 41 | TETA(Orn)4[Dab(Dab)2]4 | 1st | | Orn | | | Orn | |
| | | 2nd | α | | δ | α | | δ |
| | | | Dab | | Dab | Dab | | Dab |
| 43 | TETA(PheOrn)2[Orn(Orn)2]2 | 1st | | Orn | | | Phe | |
| | | 2nd | α | | δ | | α | |
| | | | Orn | | Orn | | Orn | |
| 44 | TETA(PheOrn)2(δOrnOrn)2 | 1st | | Orn | | | Phe | |
| | | 2nd | α | | δ | | α | |
| | | | | | Orn | | Orn | |
| 46 | TETA(DPheOrn)2(OrnOrn)2 | 1st | | Orn | | | D-Phe | |
| | | 2nd | α | | δ | | α | |
| | | | Orn | | | | Orn | |
| 47 | TETA(TrpOrn)2(OrnOrn)2 | 1st | | Orn | | | Trp | |
| | | 2nd | α | | δ | | α | |
| | | | Orn | | | | Orn | |
| 49 | TETA(Phe-αOrnOrn)4 | 1st | | Phe | | | Phe | |
| | | 2nd | | α | | | α | |
| | | | | Orn | | | Orn | |
| | | 3rd | α | δ | | α | δ | |
| | | | Orn | | | Orn | | |
| 50 | TETA(OrnOrn)2(PheOrn)2 | 1st | | Phe | | | Orn | |
| | | 2nd | | α | | α | | δ |
| | | | | Orn | | Orn | | |
| 51 | TETA[Phe-Orn(Orn)2]4 | 1st | | Phe | | | Phe | |
| | | 2nd | | α | | | α | |
| | | | | Orn | | | Orn | |
| | | 3rd | α | δ | | α | δ | |
| | | | Orn | Orn | | Orn | Orn | |

TABLE 5

| | | | TETA | | | |
|---|---|---|---|---|---|---|
| Example | | | 1 | | 4 | |
| 52 | TETA(TyrOrn) | 1st | Tyr | | Orn | |
| | 2(OrnOrn)2 | 2nd | α | | α | δ |
| | | | Orn | | Orn | |
| 53 | TETA(Dab)4 | 1st | Dab | | Dab | |
| | [Dab(Dab)2]4 | 2nd | α | δ | α | δ |
| | | | Dab | Dab | Dab | Dab |
| 54 | TETA(Lys)4 | 1st | Lys | | Lys | |
| | [Dab(Dab)2]4 | 2nd | α | ε | α | ε |
| | | | Dab | Dab | Dab | Dab |
| 55 | TETA(D-Phe- | 1st | D-Phe | | D-Phe | |
| | αOrnOrn)4 | 2nd | α | | α | |
| | | | Orn | | Orn | |
| | | 3rd | α | δ | α | δ |
| | | | Orn | | Orn | |
| 56 | TETA(TrpOrn | 1st | Trp | | Trp | |
| | Dab)4 | 2nd | α | | α | |
| | | | Orn | | Orn | |
| | | 3rd | α | δ | α | δ |
| | | | Dab | | Dab | |
| 57 | TETA(PheOrn)4 | 1st | Phe | | Phe | |
| | | 2nd | α | | α | |
| | | | Orn | | Orn | |
| 58 | TETA(OrnOrn) | 1st | Orn | | Cha | |
| | 2(ChaOrn)2 | 2nd | α | δ | α | |
| | | | Orn | | Orn | |
| 59 | TETA(Orn)2 | 1st | Orn | | Dab | |
| | [Dab(Dab)2]2 | 2nd | α | δ | α | γ |
| | | | | | Dab | Dab |
| 62 | TETA(4- NH2Bz)4[Orn (Orn)2]4 | 1st | 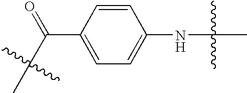 | | 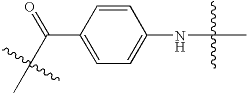 | |
| | | 2nd | Orn | | Orn | |
| | | 3rd | α | δ | α | δ |
| | | | Orn | Orn | Orn | Orn |
| 63 | TETA(PheDab)4 | 1st | Phe | | Phe | |
| | | 2nd | α | | α | |
| | | | Dab | | Dab | |
| 65 | TETA(OrnOrnOrn)2 | 1st | Orn | | Pro | |
| | (ProOrnOrn)2 | 2nd | α | δ | α | |
| | | | Orn | | Orn | |
| | | 3rd | α | δ | α | δ |
| | | | Orn | | Orn | Orn |
| | | | TETA | | | |
| | | | 7 | | 10 | |
| 52 | TETA(TyrOrn) | 1st | Orn | | Tyr | |
| | 2(OrnOrn)2 | 2nd | α | δ | α | |
| | | | Orn | | Orn | |
| 53 | TETA(Dab)4 | 1st | Dab | | Dab | |
| | [Dab(Dab)2]4 | 2nd | α | δ | α | δ |
| | | | Dab | Dab | Dab | Dab |
| 54 | TETA(Lys)4 | 1st | Lys | | Lys | |
| | [Dab(Dab)2]4 | 2nd | α | ε | α | ε |
| | | | Dab | Dab | Dab | Dab |
| 55 | TETA(D-Phe- | 1st | D-Phe | | D-Phe | |
| | αOrnOrn)4 | 2nd | α | | α | |
| | | | Orn | | Orn | |
| | | 3rd | α | δ | α | δ |
| | | | Orn | | Orn | |
| 56 | TETA(TrpOrn | 1st | Trp | | Trp | |
| | Dab)4 | 2nd | α | | α | |
| | | | Orn | | Orn | |
| | | 3rd | α | δ | α | δ |
| | | | Dab | | Dab | |
| 57 | TETA(PheOrn)4 | 1st | Phe | | Phe | |
| | | 2nd | α | | α | |
| | | | Orn | | Orn | |
| 58 | TETA(OrnOrn) | 1st | Cha | | Orn | |
| | 2(ChaOrn)2 | 2nd | α | | α | δ |
| | | | Orn | | Orn | |
| 59 | TETA(Orn)2 | 1st | Dab | | Orn | |
| | [Dab(Dab)2]2 | 2nd | α | γ | α | δ |
| | | | Dab | Dab | | |

| | | | | |
|---|---|---|---|---|
| 62 | TETA(4- NH2Bz)4[Orn (Orn)2]4 | 1st | 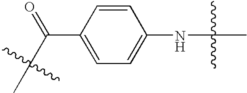 | 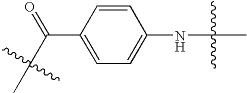 |
| | | 2nd | Orn | Orn |

| | | | | |
|---|---|---|---|---|
| 3rd | α | δ | α | δ |
| | Orn | Orn | Orn | Orn |

| 63 | TETA(PheDab)4 | 1st | Phe | Phe | |
|---|---|---|---|---|---|
| | | 2nd | α | α | |
| | | | Dab | Dab | |
| 65 | TETA(OrnOrnOrn)2 | 1st | Pro | Orn | |
| | (ProOrnOrn)2 | 2nd | α | α | δ |
| | | | Orn | Orn | |

| | | | | |
|---|---|---|---|---|
| 3rd | α | δ | α | δ |
| | Orn | Orn | Orn | |

TABLE 6

| | | | TETA | | | |
|---|---|---|---|---|---|---|
| Example | | | 1 | | 4 | |
| 66 | TETA(ProOrnOrn) | 1st | Pro | | Orn | |
| | 2(OrnOrnOrn)2 | 2nd | α | | α | δ |
| | | | Orn | | Orn | |
| | | 3rd | α | δ | α | δ |
| | | | Orn | | Orn | |
| 67 | TETA(PheOrn | 1st | Phe | | Phe | |
| | Dab)4 | 2nd | α | | α | |
| | | | Orn | | Orn | |
| | | 3rd | α | δ | α | δ |
| | | | Dab | | Dab | |
| 68 | TETA(PheOrnb- | 1st | Phe | | Phe | |
| | Ala)4 | 2nd | α | | α | |
| | | | Orn | | Orn | |
| | | 3rd | α | δ | α | δ |
| | | | b-Ala | | b-Ala | |
| 69 | TETA(OrnPhe | 1st | Orn | | Orn | |
| | Dab)4 | 2nd | α | δ | α | δ |
| | | | Phe | | Phe | |
| | | 3rd | α | | α | |
| | | | Dab | | Dab | |
| 70 | TETA(OrnPhe | 1st | Orn | | Orn | |
| | Orn)4 | 2nd | α | δ | α | δ |
| | | | Phe | | Phe | |
| | | 3rd | α | | α | |
| | | | Orn | | Orn | |
| 73 | TETA(OrnOrn) | 1st | Orn | | Leu | |
| | 2(LeuOrn)2 | 2nd | α | δ | α | |
| | | | Orn | | Orn | |
| 74 | TETA[PheAsp | 1st | Phe | | Phe | |
| | (DETA)]4 | | α | | α | |
| | | 2nd, 3rd | 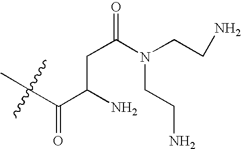 | | 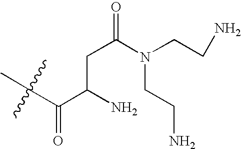 | |
| 75 | TETA(LeuOrn)4 | 1st | Leu | | Leu | |
| | | 2nd | α | | α | |
| | | | Orn | | Orn | |
| | | | TETA | | | |
| | | | 7 | | 10 | |
| 66 | TETA(ProOrnOrn) | 1st | Orn | | Pro | |
| | 2(OrnOrnOrn)2 | 2nd | α | δ | α | |
| | | | Orn | | Orn | |
| | | 3rd | α | δ | α | δ |
| | | | Orn | | Orn | |
| 67 | TETA(PreOrn | 1st | Phe | | Phe | |
| | Dab)4 | 2nd | α | | α | |
| | | | Orn | | Orn | |
| | | 3rd | α | δ | α | δ |
| | | | Dab | | Dab | |
| 68 | TETA(PheOrnb- | 1st | Phe | | Phe | |
| | Ala)4 | 2nd | α | | α | |
| | | | Orn | | Orn | |
| | | 3rd | α | δ | α | δ |
| | | | b-Ala | | b-Ala | |
| 69 | TETA(OrnPhe | 1st | Orn | | Orn | |
| | Dab)4 | 2nd | α | δ | α | δ |
| | | | Phe | | Phe | |
| | | 3rd | α | | α | |
| | | | Dab | | Dab | |
| 70 | TETA(OrnPhe | 1st | Orn | | Orn | |
| | Orn)4 | 2nd | α | δ | α | δ |
| | | | Phe | | Phe | |
| | | 3rd | α | | α | |
| | | | Orn | | Orn | |

| 73 | TETA(OrnOrn) | 1st | Leu | Orn | |
|---|---|---|---|---|---|
| | 2(LeuOrn)2 | 2nd | α | α | δ |
| | | | Orn | Orn | |

| | | | | |
|---|---|---|---|---|
| 74 | TETA[PheAsp | 1st | Phe | Phe |
| | (DETA)]4 | | α | α |
| | | 2nd, 3rd | 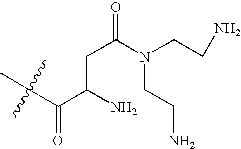 | 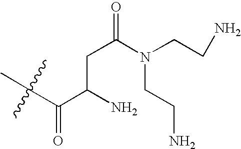 |
| 75 | TETA(LeuOrn)4 | 1st | Leu | Leu |
| | | 2nd | α | α |
| | | | Orn | Orn |

TABLE 7

| | | | TEPA | | | | | |
|---|---|---|---|---|---|---|---|---|
| Example | | | 1 | | 4 | | 7 | |
| 45 | TEPA(Phe-αOrnOrn)5 | 1st | | Phe | | Phe | | Phe |
| | | 2nd | | α | | α | | α |
| | | | | Orn | | Orn | | Orn |
| | | 3rd | α | δ | α | δ | α | δ |
| | | | Orn | | Orn | | Orn | |
| 64 | TEPA(PheOrn)5 | 1st | | Phe | | Phe | | Phe |
| | | 2nd | | α | | α | | α |
| | | | | Orn | | Orn | | Orn |

| | | | TEPA | | | |
|---|---|---|---|---|---|---|
| Example | | | 10 | | 13 | |
| 45 | TEPA(Phe-αOrnOrn)5 | 1st | | Phe | | Phe |
| | | 2nd | | α | | α |
| | | | | Orn | | Orn |
| | | 3rd | α | δ | α | δ |
| | | | Orn | | Orn | |
| 64 | TEPA(PheOrn)5 | 1st | | Phe | | Phe |
| | | 2nd | | α | | α |
| | | | | Orn | | Orn |

TABLE 8

| Exam- | | | TAA | | |
|---|---|---|---|---|---|
| ple | | | 1 | 2 | 3 |
| 48 | TAA(-Phe | 1st | Phe | Phe | Phe |
| | OrnOrn)3 | 2nd | α | α | α |
| | | | Orn | Orn | Orn |

| | | | | | | |
|---|---|---|---|---|---|---|
| 3rd | α | δ | α | δ | α | δ |
| | Orn | | Orn | | Orn | |

| | | | | | |
|---|---|---|---|---|---|
| 60 | TAA(Trp | 1st | Trp | Trp | Trp |
| | OrnOrn)3 | 2nd | α | α | α |
| | | | Orn | Orn | Orn |

| | | | | | | |
|---|---|---|---|---|---|---|
| 3rd | α | δ | α | δ | α | δ |
| | Orn | | Orn | | Orn | |

| | | | | | |
|---|---|---|---|---|---|
| 61 | TAA(Trp | 1st | Trp | Trp | Trp |
| | OrnDab)3 | 2nd | α | α | α |
| | | | Orn | Orn | Orn |

| | | | | | | |
|---|---|---|---|---|---|---|
| 3rd | α | δ | α | δ | α | δ |
| | Dab | | Dab | | Dab | |

| | | | | | |
|---|---|---|---|---|---|
| 71 | TAA[PheDAb | 1st | Phe | Phe | Phe |
| | (Dab)2]3 | 2nd | α | α | α |
| | | | Dab | Dab | Dab |

| | | | | | | |
|---|---|---|---|---|---|---|
| 3rd | α | γ | α | γ | α | γ |
| | Dab | Dab | Dab | Dab | Dab | Dab |

| | | | | | |
|---|---|---|---|---|---|
| 72 | TAA[3,5-NH2Bz (Orn)2]3 | 1st | 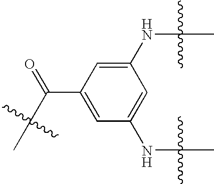 | 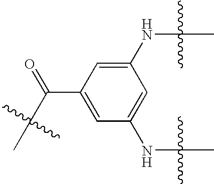 | 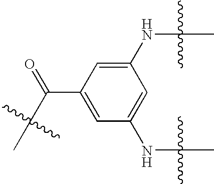 |

| | | | | | | |
|---|---|---|---|---|---|---|
| 2nd | Orn | Orn | Orn | Orn | Orn | Orn |

| | | | | | |
|---|---|---|---|---|---|
| 76 | TAA[(benzoyl- 4-methylene) DETA]3 | 1st | 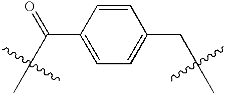 | 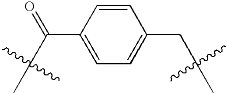 | 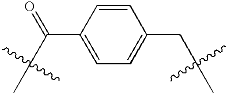 |
| | | 2nd | 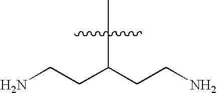 | 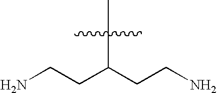 | 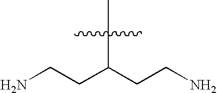 |

### Example 77

### Evaluation of Efficacy of Compounds Synthesized Above as Phosphorus Absorption Inhibitors

Seven-week-old male rats of SD line (available from SANKYO Labo-Service Co., Ltd.), as test animals, were kept and acclimatized using the usual feed (Solid CRF-1 available from ORIENTAL Yeast Co., Ltd.) and they were used in the following tests after they were fasted overnight. Each candidate substance (synthesized compound) dissolved in distilled water for injection (Otsuka Pharmaceutical Co., Ltd.) was orally administered in a dose of 100 mg/5 mL/kg or 50 mg/5 mL/kg and after 5 minutes from the administration, 3 mg/3 mL/kg, 1 MBq/kg of an NaH₂PO₄ solution containing ³²P (available from PerkinElmer Company) was orally administered. Subsequently, the blood was collected with the elapse of time through the caudal vein of the test animal to thus obtain the serum thereof and the radioactivity of 40 μL of the serum was determined using a liquid scintillation counter. The inhibitory rate (%) for each candidate compound was calculated on the basis of the foregoing measured values according to the following equation:

Inhibitory Rate (%)=100×{1−[ACU of Candidate Substance−Administered Group (0˜120 min)]/[AUC of the Control Group (0˜120 min)]}

The results obtained using the candidate substance (synthesized compound) in a dose of 100 mg/5 mL/kg are summarized in the following Table A, while those observed using the candidate substance (synthesized compound) in a dose of 50 mg/5 mL/kg are summarized in the following Table B.

### Example 78

### Evaluation of Cytotoxicity of Synthesized Compounds

MDCK Cells were inoculated into wells of a 96-well plate filled with D-MEM/F12 culture medium (available from GIBCO Company) supplemented with 10% FBS (available from TISSUE CULTURE BIOLOGICALS Company). After the cultivation of the cells for 3 days in a CO₂ incubator, the culture medium was removed, a culture medium, in which each candidate substance (synthesized compound) had been dissolved, was added, followed by the cultivation for one day. To each well, there was added 10 μL of Cell Count Reagent SF (available from NAKARAI TESUKU K.K.), followed by the cultivation thereof for 2 hours and the subsequent determination of the absorbance observed at a wavelength of 460 nm. The IC50 values each were calculated on the basis of these measured values.

The results thus obtained are summarized in the following Table A.

TABLE A

| | Inhibitory Rate (%) of Phosphorus | Cytotoxicity |
|---|---|---|
| Ex. No. | Absorption | MDCK (IC50, mg/mL) |
| 2 | 52 | >10 |
| 4 | 58 | >10 |
| 5 | 52 | >10 |
| 6 | 30 | >10 |
| 7 | 24 | >10 |
| 8 | 63 | |
| 9 | 30 | |
| 10 | 30 | |
| 11 | 44 | |
| 12 | 43 | >10 |
| 13 | 36 | >10 |
| 14 | 46 | >10 |
| 15 | 33 | >10 |
| 16 | 49 | >10 |
| 17 | 30 | |
| 19 | 35 | |
| 20 | 60 | >10 |
| 22 | 60 | |
| 23 | 45 | |
| 27 | 64 | |
| 28 | 66 | >10 |
| 29 | 55 | >10 |

TABLE B

| Ex. | 50 mg/kg |
|---|---|
| No. | Inhibition (%) |
| 34 | 63 |
| 35 | 58 |
| 36 | 55 |
| 37 | 60 |
| 38 | 63) |
| 39 | 55) |
| 40 | 76 |
| 41 | 58) |
| 42 | 59 |
| 43 | 60 |
| 44 | 52 |
| 45 | 76 |
| 46 | 62 |
| 47 | 72 |
| 48 | 74 |
| 49 | 74 |
| 50 | 79 |
| 51 | 62 |
| 52 | 59) |
| 53 | 60 |
| 54 | 53 |
| 55 | 85 |
| 56 | 71 |
| 57 | 69 |
| 58 | 79 |
| 59 | 57 |
| 60 | 64 |
| 61 | 70 |
| 62 | 66 |
| 63 | 73 |
| 64 | 87 |
| 65 | 57 |
| 66 | 56 |
| 67 | 84 |
| 68 | 73 |
| 69 | 67 |
| 70 | 57 |
| 71 | 54 |
| 72 | 79 |
| 73 | 52 |
| 74 | 79 |
| 75 | 55 |
| 76 | 78 |

What is claimed is:

1. A water-soluble compound represented by the following general formula (I) or a salt thereof: 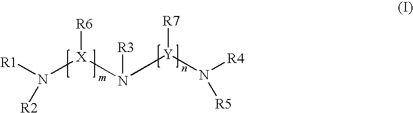
In the formula (I), R1 to R7 may be the same or different and each represent a hydrogen atom, an amidino group, a substituted or unsubstituted aminoalkyl group, a substituted or unsubstituted alkyl group, or a group: Rx-[A]p- (wherein A represents a connecting group; p represents 0 or 1; and Rx represents an amino-carboxylic acid residue, a substituted or unsubstituted aminoalkyl group, a substituted or unsubstituted alkyl group, or a group represented by the following formula (II): 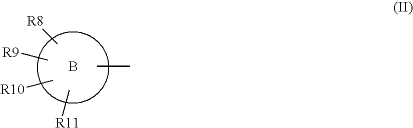
In Formula (II), B represents an aryl group, a heteroaryl group or a cycloalkyl group, R8 to R11 may be the same or different and each represent a hydrogen atom, an amidino group, an amino group, a substituted or unsubstituted aminoalkyl group, a substituted or unsubstituted alkyl group, or a group: Ry-[A1]q- (wherein A1 represents a connecting group; q represents 0 or 1; and Ry represents an amino-carboxylic acid residue, a substituted or unsubstituted aminoalkyl group, or a substituted or unsubstituted alkyl group,
X and Y may be the same or different and each represent —N— or —CH—, m and n may be the same or different and each represent an integer ranging from 2 to 7, provided that when m is 2, X represents —CH—, when m is not less than 3, a plurality of groups X—R6 may be the same or different, but is not adjacent to N—R6, and
When n is 2, Y represents —CH— and when n is not less than 3, a plurality of groups Y—R7 may be the same or different, but is not adjacent to N—R7.

2. The water-soluble compound or a salt thereof as set forth in claim 1, wherein in the formula (I), R1 to R7 may be the same or different and each represent a hydrogen atom, an amidino group, a substituted or unsubstituted aminoalkyl group, a substituted or unsubstituted alkyl group, or a group: Rx-[A]p- (wherein Rx represents an amino-carboxylic acid residue, A represents a connecting group; and p represents 0 or 1).

3. The compound as set forth in claim 2, wherein one of R1 and R2 represents a hydrogen atom and the other represents a group: Rx-[A]p-; and one of R4 and R5 represents a hydrogen atom and the other represents a group: Rx-[A]p-.

4. The compound as set forth in claim 2, wherein the plurality of groups: —X(R6)- may be the same or different from one another and represent a combination of groups —CH(R6)- and —N(R6)-.

5. The compound as set forth in claim 2, wherein the plurality of groups: —Y(R7)- may be the same or different from one another and represent a combination of groups —CH(R7)- and —N(R7)-.

6. The compound as set forth in claim 2, wherein the plurality of groups: —X(R6)- may be the same or different from one another and represent a combination of groups —CH(R6)- and —N(R6)-; and the plurality of groups: —Y(R7)- may be the same or different from one another and represent a combination of groups —CH(R7)- and —N(R7)- and wherein the number of —N-present in the backbone and originated from the groups: —X(R6)- and —Y(R7)- ranges from 1 to 4.

7. The compound as set forth in claim 2, wherein the plurality of groups: —X(R6)- represent —CH(R6)- groups, which may be the same or different and the plurality of groups: —Y(R7)- represent —CH(R7)- groups, which may be the same or different.

8. The compound as set forth in claim 2, wherein the group Rx represents an amino acid residue and p represents 0.

9. The compound as set forth in claim 8, wherein the amino acid residue is a poly(amino acid) residue.

10. The compound as set forth in claim 8, wherein the amino acid residue is one member or a mixture of at least two members selected from the group consisting of lysine, arginine, ornithine, and glycine.

11. The water-soluble compound or a salt thereof as set forth in claim 1 or 2, wherein all of the substituents R1 to R7 do not simultaneously represent a hydrogen atom.

12. The water-soluble compound or a salt thereof as set forth in claim 1, wherein at least one of R1 to R7 represents Rx-[A]ₚ- wherein the aminocarboxylic acid residue represented by Rx is a poly(amino acid) residue comprising an aminocarboxylic acid residue carrying an aryl group or a heteroaryl group and at least one acyclic amino acid residue, combined with the former, selected from the group consisting of basic aliphatic amino acid residues, aliphatic mono-aminocarboxylic acid residues and acidic amino acid residues.

13. The water-soluble compound or a salt thereof as set forth in claim 12, wherein the aminocarboxylic acid carrying an aryl group or a heteroaryl group is phenylalanine or tryptophane; the aminocarboxylic acid carrying a cycloalkyl group is cyclohexyl-alanine; the external end of the poly(amino acid) residue is one other than phenylalanine residue, tryptophane residue and cyclohexyl-alanine residue.

14. The water-soluble compound or a salt thereof as set forth in claim 1, wherein at least one of the substituents R1 to R7 represents a group: Rx-[A]ₚ- wherein Rx represents a group represented by Formula (II) wherein B represents an aryl group, at least one of the substituents R8 to R11 represents a group: Ry-[A1]_{q}- wherein A1 represents a connecting group, q represents 0 or 1, and Ry represents an aminocarboxylic acid residue or a substituted or unsubstituted aminoalkyl group and p represents 1.

15. An agent for inhibiting the absorption of phosphorus comprising a compound or a salt thereof as set forth in claim 1.

16. An agent for inhibiting the absorption of phosphorus comprising a compound or a salt thereof as set forth in claim 2.

17. The agent for inhibiting the absorption of phosphorus as set forth in claim 16, wherein it is in an orally administrable form.

18. The agent for inhibiting the absorption of phosphorus as set forth in claim 17, wherein it is in the form of a medical or pharmaceutical product.

19. The agent for inhibiting the absorption of phosphorus as set forth in claim 17, wherein it is in the form of a food.
